(19) United States
(12) Patent Application Publication
(10) Pub. No.: US 2016/0271189 A1
(43) Pub. Date: Sep. 22, 2016

(54) METHODS AND COMPOSITIONS RELATING TO MICROBIAL TREATMENT AND DIAGNOSIS OF SKIN DISORDERS

(71) Applicant: Whole Biome, Inc., San Francisco, CA (US)

(72) Inventors: Colleen CUTCLIFFE, Menlo Park, CA (US); John S. Eid, San Francisco, CA (US); James H. Bullard, San Francisco, CA (US); Marcus F. SCHICKLBERGER, Richmond, CA (US); Andrew T. CHENG, San Mateo, CA (US)

(21) Appl. No.: 15/074,923

(22) Filed: Mar. 18, 2016

Related U.S. Application Data
(60) Provisional application No. 62/134,947, filed on Mar. 18, 2015.

(51) Int. Cl. A61K 35/74; A61K 31/715; A61K 38/48; A61K 33/40; A61K 9/00; A61K 31/4172

(52) U.S. Cl. CPC A61K 35/74; A61K 9/0053; A61K 9/0014; A61K 31/4172; A61K 38/482; A61K 33/40; A61K 31/715

(57) ABSTRACT

The present disclosure provides methods, systems, compositions, and kits to address the need for microbiome-related treatment of health conditions and disease. The disclosure provides compositions and methods for the treatment of skin disorders using microbial compositions to a subject.

### CROSS REFERENCE

This application claims the benefit of U.S. Provisional Patent Application No. 62/134,947, filed on Mar. 18, 2015, which is incorporated herein by reference in its entirety.

### BACKGROUND OF THE INVENTION

The skin of a subject is inhabited by a highly diverse population of microorganisms, referred to as the microbiome. The skin microbiome can play a key role in maintaining the cutaneous immune system. Additionally inflammation disorders as well as other allergic reactions can be caused by dysbiosis in the gut microbiome. So, impairment of either the skin or gut microbiomes, or combination thereof, can lead to various skin conditions and disorders. Consequently, development of microbiome therapeutic and diagnostic applications for treating microbiome-related skin conditions and disorders can be necessary.

### SEQUENCE LISTING

The instant application contains a Sequence Listing which has been submitted in ASCII format via EFS-Web and is hereby incorporated by reference in its entirety. Said ASCII copy, created on Mar. 17, 2016, is named 46790-705.201.txt and is 36,254,267 bytes in size.

### SUMMARY OF THE INVENTION

In some embodiments, the disclosure provides a method of treating a skin disorder in a subject in need thereof, the method comprising administering to the subject a pharmaceutical composition comprising a therapeutically-effective amount of a population of isolated and purified microorganisms. In some embodiments, the pharmaceutical composition comprises a pharmaceutically-acceptable carrier. In some embodiments, at least one of the microorganisms is capable of modulating a pH of the subject. In some embodiments, the at least one microorganism modulates the pH of the skin of the subject. In some embodiments, the skin disorder is atopic dermatitis. In some embodiments, the subject is a child. In some embodiments, the subject is human. In some embodiments, at least one of the microorganisms comprises a microorganism selected from the group consisting of: Akkermansia muciniphila, Anaerostipes caccae, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium coccoides, Clostridium indolis, Clostridium nexile, Clostridium orbiscindens, Clostridium propionicum, Clostridium xylanolyticum, Enterococcus faecium, Eubacterium hallii, Eubacterium rectale, Faecalibacterium prausnitzii, Fibrobacter succinogenes, Lactobacillus acidophilus, Lactobacillus brevis, Lactobacillus bulgaricus, Lactobacillus casei, Lactobacillus caucasicus, Lactobacillus fermentum, Lactobacillus helveticus, Lactobacillus lactis, Lactobacillus plantarum, Lactobacillus reuteri, Lactobacillus rhamnosus, Oscillospira guilliermondii, Roseburia cecicola, Roseburia inulinivorans, Ruminococcus flavefaciens, Ruminococcus gnavus, Ruminococcus obeum, Stenotrophomonas nitritireducens, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus, Coprococcus eutactus, Eubacterium cylindroides, Eubacterium dolichum, Eubacterium ventriosum, Roseburia faeccis, Roseburia hominis, Roseburia intestinalis, Lacatobacillus bifidus, Lactobacillus johnsonii, and any combination thereof. In some embodiments, the Lactobacillus reuteri is selected from the group consisting of: Lactobacillus reuteri RC-14, Lactobacillus reuteri L22, and any combination thereof. In some embodiments, at least one of the microorganisms is a microorganism capable of producing vinegar. In some embodiments, the microorganism capable of producing vinegar is an acetic acid bacterium. In some embodiments, the at least one of said microorganisms is a microorganism capable of producing hydrogen peroxide. In some embodiments, the treating results in an altered microbiome in said subject. In some embodiments, the treating results in a reduction of a pH of said subject. In some embodiments, the reduction comprises reducing said pH to between about pH 4.0 and about pH 5.0. In some embodiments, the pharmaceutical composition is formulated for oral administration. In some embodiments, the pharmaceutical composition is formulated for topical administration. In some embodiments, the pharmaceutical composition is formulated as a pill. In some embodiments, the pharmaceutical composition is formulated as a capsule. In some embodiments, the pharmaceutical composition is formulated as a patch. In some embodiments, the pharmaceutical composition further comprises hydrogen peroxide. In some embodiments, the pharmaceutical composition further comprises vinegar. In some embodiments, the pharmaceutical composition further comprises trans-urocanic acid. In some embodiments, the pharmaceutical composition further comprises a metabolite. In some embodiments, the metabolite is selected from the group consisting of acetate, propionate, isobutyrate, isovaleric acid, 3-methylbutanoic acid, valeric acid, pentanoic acid, delphinic acid, isopentanoic acid, and butyrate. In some embodiments, the pharmaceutical composition further comprises anti-microbial peptide. In some embodiments, the anti-microbial peptide is a phenol-soluble modulin (PSM). In some embodiments, the phenol-soluble modulin is PSM gamma. In some embodiments, the phenol-soluble modulin is PSM delta. In some embodiments, the pharmaceutical composition further comprises bacteriocin. In some embodiments, the bacteriocin is selected from the group consisting of: epidermin, epilancin K7, epilancin 15×, pep5, staphylococcin 1580, and any combination thereof. In some embodiments, at least one of said microorganisms comprises a microorganism that stimulates a toll-like receptor. In some embodiments, the pharmaceutical composition further comprises an enzyme. In some embodiments, the enzyme is a serine protease. In some embodiments, the pharmaceutical composition further comprises a probiotic. In some embodiments, the pharmaceutical composition further comprises a prebiotic. In some embodiments, the prebiotic is selected from the group consisting of: complex carbohydrates, complex sugars, resistant dextrins, resistant starch, amino acids, peptides, nutritional compounds, biotin, polydextrose, oligosaccharides, polysaccharide, fructooligosaccharide (FOS), fructans, soluble fiber, insoluble fiber, fiber, starch, galactooligosaccharides (GOS), inulin, lignin, psyllium, chitin, chitosan, gums, high amylose cornstarch (HAS), cellulose, β-glucans, hemi-celluloses, lactulose, mannooligosaccharides, mannan oligosaccharides (MOS), oligofructose-enriched inulin, oligofructose, oligodextrose, tagatose, trans-galactooligosaccharide, pectin, resistant starch, and xylooligosaccharides (XOS), and any combination thereof. In some embodiments, the method further comprises determining a microbiome profile of the subject. In some embodiments, the method further comprises diagnosing a condition of the subject based on the microbiome profile. In some embodiments, at least one of said microorganisms comprises a microorganism that is a recombinant microorganism.

In some embodiments, the invention provides a pharmaceutical composition comprising a therapeutically effective amount of a population of isolated and purified microorganisms. In some embodiments, at least one of said microorganisms comprises a microorganism that modulates pH. In some embodiments, at least one of said microorganisms comprises a microorganism that modulates a gut microbiome. In some embodiments, at least one of said microorganisms comprises a microorganism that modulates butyrate production. In some embodiments, at least one of said microorganism comprises a microorganism selected from the group consisting of: Akkermansia muciniphila, Anaerostipes caccae, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium coccoides, Clostridium indolis, Clostridium nexile, Clostridium orbiscindens, Clostridium propionicum, Clostridium xylanolyticum, Enterococcus faecium, Eubacterium hallii, Eubacterium rectale, Faecalibacterium prausnitzii, Fibrobacter succinogenes, Lactobacillus acidophilus, Lactobacillus brevis, Lactobacillus bulgaricus, Lactobacillus casei, Lactobacillus caucasicus, Lactobacillus fermentum, Lactobacillus helveticus, Lactobacillus lactis, Lactobacillus plantarum, Lactobacillus reuteri, Lactobacillus rhamnosus, Oscillospira guilliermondii, Roseburia cecicola, Roseburia inulinivorans, Ruminococcus flavefaciens, Ruminococcus gnavus, Ruminococcus obeum, Stenotrophomonas nitritireducens, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus, Coprococcus eutactus, Eubacterium cylindroides, Eubacterium dolichum, Eubacterium ventriosum, Roseburia faeccis, Roseburia hominis, Roseburia intestinalis, Lacatobacillus bifidus, Lactobacillus johnsonii, and any combination thereof. In some embodiments, said Lactobacillus reuteri is Lactobacillus reuteri RC-14. In some embodiments, said Lactobacillus reuteri is Lactobacillus reuteri L22. In some embodiments, the pharmaceutical composition has a pH of about pH 5.5. In some embodiments, at least one of said microorganisms is a microorganism capable of producing vinegar. In some embodiments, the microorganism capable of producing vinegar is an acetic acid bacterium. In some embodiments, at least one of said microorganisms is a microorganism capable of producing hydrogen peroxide. In some embodiments, said composition results in a reduction of said pH. In some embodiments, said pH is reduced to a range between about pH 4.0 and about pH 5.0. In some embodiments, said composition is formulated as a pill, a tablet, or a capsule. In some embodiments, said composition is formulated as a lotion or a cream. In some embodiments, said composition is formulated as a patch or a wrap. In some embodiments, said composition further comprises hydrogen peroxide. In some embodiments, said composition further comprises vinegar. In some embodiments said composition further comprises trans-urocanic acid. In some embodiments said composition further comprises a metabolite. In some embodiments said metabolite is selected from the group consisting of acetate, propionate, isobutyrate, isovaleric acid, 3-methylbutanoic acid, valeric acid, pentanoic acid, delphinic acid, isopentanoic acid, and butyrate. In some embodiments said composition further comprises an anti-microbial peptide. In some embodiments said anti-microbial peptide is a phenol-soluble modulin (PSM). In some embodiments said phenol-soluble modulin is PSM gamma. In some embodiments said phenol-soluble modulin is PSM delta. In some embodiments said composition further comprises a bacteriocin. In some embodiments said bacteriocin is selected from the group consisting of: epidermin, epilancin K7, epilancin 15×, pep5, staphylococcin 1580, and any combination thereof. In some embodiments said composition further comprises an enzyme. In some embodiments said enzyme is a serine protease. In some embodiments said composition further comprises a prebiotic. In some embodiments said prebiotic is selected from the group consisting of: complex carbohydrates, complex sugars, resistant dextrins, resistant starch, amino acids, peptides, nutritional compounds, biotin, polydextrose, oligosaccharides, polysaccharide, fructooligosaccharide (FOS), fructans, soluble fiber, insoluble fiber, fiber, starch, galactooligosaccharides (GOS), inulin, lignin, psyllium, chitin, chitosan, gums, high amylose cornstarch (HAS), cellulose, β-glucans, hemi-celluloses, lactulose, mannooligosaccharides, mannan oligosaccharides (MOS), oligofructose-enriched inulin, oligofructose, oligodextrose, tagatose, trans-galactooligosaccharide, pectin, resistant starch, and xylooligosaccharides (XOS), and any combination thereof. In some embodiments said prebiotic is an oligosaccharide. In some embodiments said prebiotic is inulin. In some embodiments said composition further comprises a pharmaceutically-acceptable carrier In some embodiments the population of isolated and purified microbes comprises a microbe with a ribosomal RNA (rRNA) sequence comprising at least about 85% sequence identity to a rRNA sequence selected from the group consisting of: Akkermansia muciniphila, Bifidobacterium adolescentis, Bifidobacterium infantis, Bifidobacterium longum, Clostridium beijerinckii, Clostridium butyricum, Clostridium indolis, Eubacterium hallii, Faecalibacterium prausnitzii, and any combination thereof. In some embodiments the population of isolated and purified microbes comprises a microbe with a ribosomal RNA (rRNA) sequence comprising at least about 85% sequence identity to a rRNA sequence selected from the group consisting of: Lactobacillus reuteri, Streptococcus mutans, Stenotrophomonas nitritireducens, and any combination thereof. In some embodiments the population of isolated and purified microbes comprises a microbe with a ribosomal RNA (rRNA) sequence comprising at least about 85% sequence identity to a rRNA sequence selected from the group consisting of: Lactobacillus rhamnosus, Faecalibacterium prausnitzii, Oscillospira guilliermondii, Clostridium orbiscindens, Clostridium colinum, Clostridium aminophilum, and Ruminococcus obeum, and any combination thereof.

In some embodiments, the invention provides a method of treating a skin disorder in a subject in need thereof, the method comprising: administering to the subject a pharmaceutical composition comprising a therapeutically-effective amount of a population of isolated and purified microbes. In some embodiments, at least one of said isolated and purified microbes comprises a microbe that alters a pH in the subject. In some embodiments, at least one of said isolated and purified microbes comprises a microbe that restores a gut microbiome of the subject. In some embodiments, the population of isolated and purified microbes comprises a microbe with a ribosomal RNA (rRNA) sequence comprising at least about 85% sequence identity to a rRNA sequence from Lactobacillus rhamnosus. In some embodiments, the population of isolated and purified microbes comprises a microbe with a ribosomal RNA (rRNA) sequence comprising at least about 85% sequence identity to a rRNA sequence from Oscillospira guilliermondii. In some embodiments, the population of isolated and purified microbes comprises a microbe with a ribosomal RNA (rRNA) sequence comprising at least about 85% sequence identity to a rRNA sequence from Clostridium orbiscindens. In some embodiments, the population of isolated and purified microbes comprises a microbe with a ribosomal RNA (rRNA) sequence comprising at least about 85% sequence identity to a rRNA sequence from Clostridium colimum. In some embodiments, the population of isolated and purified microbes comprises a microbe with a ribosomal RNA (rRNA) sequence comprising at least about 85% sequence identity to a rRNA sequence from Clostridium aminophilum. In some embodiments, the population of isolated and purified microbes comprises a microbe with a ribosomal RNA (rRNA) sequence comprising at least about 85% sequence identity to a rRNA sequence from Ruminococcus obeum. In some embodiments, the population of isolated and purified microbes comprises a microbe with a ribosomal RNA (rRNA) sequence comprising at least about 85% sequence identity to a rRNA sequence from Akkermansia mucimphila. In some embodiments, the population of isolated and purified microbes comprises a microbe with a rRNA sequence comprising at least about 85% sequence identity to a rRNA sequence from Bifidobacterium adolescentis. In some embodiments, the population of isolated and purified microbes comprises a microbe with a rRNA sequence comprising at least about 85% sequence identity to a rRNA sequence from Bifidobacterium infantis. In some embodiments, the population of isolated and purified microbes comprises a microbe with a rRNA sequence comprising at least about 85% sequence identity to a rRNA sequence from Bifidobacterium longum. In some embodiments, the population of isolated and purified microbes comprises a microbe with a rRNA sequence comprising at least about 85% sequence identity to a rRNA sequence from Clostridium beijerinckii. In some embodiments, the population of isolated and purified microbes comprises a microbe with a rRNA sequence comprising at least about 85% sequence identity to a rRNA sequence from Clostridium butyricum. In some embodiments, the population of isolated and purified microbes comprises a microbe with a rRNA sequence comprising at least about 85% sequence identity to a rRNA sequence from Clostridium indolis. In some embodiments, the population of isolated and purified microbes comprises a microbe with a rRNA sequence comprising at least about 85% sequence identity to a rRNA sequence from Eubacterium hallii. In some embodiments, the population of isolated and purified microbes comprises a microbe with a rRNA sequence comprising at least about 85% sequence identity to a rRNA sequence from Faecalibacterium prausnitzii. In some embodiments, the population of isolated and purified microbes comprises a microbe with a rRNA sequence comprising at least about 85% sequence identity to a rRNA sequence from Lactobacillus reuteri. In some embodiments, the population of isolated and purified microbes comprises a microbe with a rRNA sequence comprising at least about 85% sequence identity to a rRNA sequence from Streptococcus mutans. In some embodiments, the population of isolated and purified microbes comprises a microbe with a rRNA sequence comprising at least about 85% sequence identity to a rRNA sequence from Stenotrophomonas nitritireducens. In some embodiments, the skin disorder is atopic dermatitis. In some embodiments, the skin disorder is inflammation. In some embodiments, the skin disorder is allergy. In some embodiments, the pharmaceutical composition is formulated for oral delivery. In some embodiments, the pharmaceutical composition is formulated as a pill. In some embodiments, the pill comprises an enteric-coating. In some embodiments, the enteric coating dissolves at a pH greater than at least about pH 6.5. In some embodiments, the pharmaceutical composition is delivered to a small intestine of the subject. In some embodiments, the pharmaceutical composition is delivered to an ileum of the small intestine of the subject. In some embodiments, the pharmaceutical composition is delivered to a large intestine of the subject. In some embodiments, the pharmaceutical composition further comprises a prebiotic. In some embodiments, the prebiotic comprises inulin. In some embodiments, the inulin is present in an amount of at least about 50 mg/ml in the pharmaceutical composition. In some embodiments, the subject is human. In some embodiments, the treating results in the subject having increased butyrate production as compared to a pre-treatment level. In some embodiments, the pharmaceutical composition is administered before food intake by the subject. In some embodiments, the pharmaceutical composition is administered with food intake by the subject. Further provided herein are methods further comprising determining a composition of a microbiome of the subject. In some embodiments, the therapeutically-effective amount of each purified and isolated microbe in the pharmaceutical composition is at least about 10⁵ colony forming units (CFU). In some embodiments, the pharmaceutical composition is administered after completion of an antibiotic regimen by the subject. In some embodiments, the pharmaceutical composition does not substantially release the population of isolated and purified microbes prior to a small intestine of the subject.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

The content of the International Nucleotide Sequence Database Collaboration (DDBJ/EMBL/GENBANK) accession number CP001071.1 for microbial strain Akkermansia muciniphila, culture collection ATCC BAA-835, is herein incorporated by reference in its entirety.

The content of DDBJ/EMBL/GENBANK accession number AJ518871.2 for microbial strain Anaerofustis stercorihominis, culture collection DSM 17244, is herein incorporated by reference in its entirety.

The content of DDBJ/EMBL/GENBANK accession number DS499744.1 for microbial strain Anaerostipes caccae, culture collection DSM 14662, is herein incorporated by reference in its entirety.

The content of DDBJ/EMBL/GENBANK accession number AJ270487.2 for microbial strain Anaerostipes caccae, butyrate-producing bacterium L1-92, is herein incorporated by reference in its entirety.

The content of DDBJ/EMBL/GENBANK accession number AY305319.1 for microbial strain Anaerostipes hadrus, butyrate-producing bacterium SS2/1, is herein incorporated by reference in its entirety.

The content of DDBJ/EMBL/GENBANK accession number AJ315980.1 for microbial strain Anaerotruncus colihominis, culture collection DSM 17241, is herein incorporated by reference in its entirety.

The content of DDBJ/EMBL/GENBANK accession number AP009256.1 for microbial strain, Bifidobacterium adolescentis, culture collection ATCC 15703, is herein incorporated by reference in its entirety.

The content of DDBJ/EMBL/GENBANK accession number CP001095.1 for microbial strain Bifidobacterium longum subsp. infantis, culture collection ATCC 15697, is herein incorporated by reference in its entirety.

The content of DDBJ/EMBL/GenBank accession number U41172.1 for microbial strain Butyrivibrio fibrisolvens, culture collection ATCC 19171, is herein incorporated by reference in its entirety.

The content of DDBJ/EMBL/GenBank accession number AJ250365.2 for microbial strain Butyrivibrio fibrisolvens, 16.4, is herein incorporated by reference in its entirety.

The content of DDBJ/EMBL/GenBank accession number U41168.1 for microbial strain Butyrivibrio fibrisolvens, OB 156, is herein incorporated by reference in its entirety.

The content of DDBJ/EMBL/GenBank accession number AY305305.1 for microbial strain Butyrate-producing bacterium, A2-232, is herein incorporated by reference in its entirety.

The content of DDBJ/EMBL/GenBank accession number AY305316.1 for microbial strain Butyrate-producing bacterium, SS3/4, is herein incorporated by reference in its entirety.

The content of DDBJ/EMBL/GENBANK accession number AE001437.1 for microbial strain Clostridium acetobutylicum, culture collection ATCC 824, is herein incorporated by reference in its entirety.

The content of DDBJ/EMBL/GENBANK accession number X78070.1 for microbial strain Clostridium acetobutylicum, culture collection DSM 792, is herein incorporated by reference in its entirety.

The content of DDBJ/EMBL/GENBANK accession number CP000721.1 for microbial strain Clostridium beijerinckii, culture collection NCIMB 8052, is herein incorporated by reference in its entirety.

The content of DDBJ/EMBL/GENBANK accession number X68189.1 for microbial strain Clostridium sporogenes, is herein incorporated by reference in its entirety.

The content of DDBJ/EMBL/GENBANK accession number X74770.1 for microbial strain Clostridium tetani, is herein incorporated by reference in its entirety.

The content of DDBJ/EMBL/GENBANK accession number AJ270491.2 for microbial strain Coprococcus, butyrate-producing bacterium L2-50, is herein incorporated by reference in its entirety.

The content of DDBJ/EMBL/GENBANK accession number EF031543.1 for microbial strain Coprococcus eutactus, culture collection ATCC 27759, is herein incorporated by reference in its entirety.

The content of DDBJ/EMBL/GenBank accession number AY305306.1 for microbial strain Eubacterium cylindroides, butyrate-producing bacterium T2-87, is herein incorporated by reference in its entirety.

The content of DDBJ/EMBL/GenBank accession number AY305313.1 for microbial strain Eubacterium cylindroides, butyrate-producing bacterium SM7/11, is herein incorporated by reference in its entirety.

The content of DDBJ/EMBL/GenBank accession number L34682.2 for microbial strain Eubacterium dolichum, culture collection DSM 3991, is herein incorporated by reference in its entirety.

The content of DDBJ/EMBL/GenBank accession number AJ270490.2 for microbial strain Eubacterium halii, butyrate-producing bacterium L2-7, is herein incorporated by reference in its entirety.

The content of DDBJ/EMBL/GenBank accession number AY305318.1 for microbial strain Eubacterium halii, butyrate-producing bacterium SM6/1, is herein incorporated by reference in its entirety.

The content of DDBJ/EMBL/GenBank accession number L34621.2 for microbial strain Eubacterium halii, culture collection ATCC 27751, is herein incorporated by reference in its entirety.

The content of DDBJ/EMBL/GenBank accession number AJ270475.2 for microbial strain Eubacterium rectale, A1-86, is herein incorporated by reference in its entirety.

The content of DDBJ/EMBL/GENBANK accession number NC_012781.1 for microbial strain Eubacterium rectale, culture collection ATCC 33656, is herein incorporated by reference in its entirety.

The content of DDBJ/EMBL/GenBank accession number L34421.2 for microbial strain Eubacterium ventriosum, culture collection ATCC 27560, is herein incorporated by reference in its entirety.

The content of DDBJ/EMBL/GENBANK accession number AY305307.1 for microbial strain Faecalibacterium prausnitzii, butyrate producing bacterium M21/2, is herein incorporated by reference in its entirety.

The content of DDBJ/EMBL/GENBANK accession number FP929046.1 for microbial strain Faecalibacterium prausnitzii is herein incorporated by reference in its entirety.

The content of DDBJ/EMBL/GENBANK accession number GG697168.2 for microbial strain Faecalibacterium prausnitzii is herein incorporated by reference in its entirety.

The content of DDBJ/EMBL/GENBANK accession number CP002158.1 for microbial strain Fibrobacter succinogenes subsp. succinogenes is herein incorporated by reference in its entirety.

The content of DDBJ/EMBL/GENBANK accession number NZ_AUJN01000001.1 for microbial strain Clostridium butyricum is herein incorporated by reference in its entirety.

The content of DDBJ/EMBL/GENBANK accession number NZ_AZUI01000001.1 for microbial strain Clostridium indolis, culture collection DSM 755, is herein incorporated by reference in its entirety.

The content of DDBJ/EMBL/GENBANK accession number ACEP01000175.1 for microbial strain Eubacterium hallii, culture collection DSM 3353, is herein incorporated by reference in its entirety.

The content of DDBJ/EMBL/GenBank accession number AY305310.1 for microbial strain Roseburia faecis, M72/1, is herein incorporated by reference in its entirety.

The content of DDBJ/EMBL/GenBank accession number AJ270482.2 for microbial strain Roseburia hominis, type strain A2-183T, is herein incorporated by reference in its entirety.

The content of DDBJ/EMBL/GenBank accession number AJ312385.1 for microbial strain Roseburia intestinalis, L1-82, is herein incorporated by reference in its entirety.

The content of DDBJ/EMBL/GenBank accession number AJ270473.3 for microbial strain Roseburia inulinivorans, type strain A2-194T, is herein incorporated by reference in its entirety.

The content of DDBJ/EMBL/GENBANK accession number NZ_ACFY01000179.1 for microbial strain Roseburia inulinivorans, culture collection DSM 16841, is herein incorporated by reference in its entirety.

The content of DDBJ/EMBL/GENBANK accession number KI912489.1 for microbial strain Ruminococcus flavefaciens, culture collection ATCC 19208, is herein incorporated by reference in its entirety.

The content of DDBJ/EMBL/GENBANK accession number AAYG02000043.1 for microbial strain Ruminococcus gnavus, culture collection ATCC 29149, is herein incorporated by reference in its entirety.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts illustrative microbiome-related health conditions and diseases for which microbiome therapeutics and diagnostics can be used. These health conditions can include: skin health, acne, atopic dermatitis, psoriasis, vaginosis, preterm delivery, allergies, preterm labor, chronic fatigue syndrome, Type 2 diabetes mellitus, depression, autism, asthma, hypertension, irritable bowel syndrome, metabolism, obesity, drug metabolism, Type I diabetes mellitus, multiple sclerosis, Clostridium difficile, inflammatory bowel disease, crohn's disease, genitourinary disorders, and heart disease.

FIG. 2 depicts an exemplary process used to identify strains related to a health condition (e.g., skin disorder; atopic dermatitis), such as to identify therapeutic consortia. Exemplary strains for treating or related to a skin disorder (e.g., atopic dermatitis) found using this method include, for example, Lactobacillus strains, such as Lactobacillus reuteri RC-14 and Lactobacillus reuteri L22, Stenotrophomonas nitritireducens, Streptococcus mutans, Lactobacillus rhamnosus, Faecalibacterium prausnitzii, Oscillospira guilliermondii, Clostridium orbiscindens, Clostridium colinum, Clostridium aminophilum, and Ruminococcus obeum. In some embodiments, isolated and purified microbial strains, for example, Lactobacillus reuteri RC-14, Lactobacillus reuteri L22, Streptococcus mutans, and Stenotrophomonas nitritireducens are formulated for topical application. In some embodiments, isolated and purified microbial strains, for example, Lactobacillus rhamnosus, Faecalibacterium prausnitzii, Oscillospira guilliermondii, Clostridium orbiscindens, Clostridium colinum, Clostridium aminophilum, and Ruminococcus obeum are formulated for oral ingestion, for example, as a pill or capsule.

FIG. 3 is an illustration depicting an exemplary platform for a Complete Biome Test (CBT) (e.g. as a diagnostic test or as a development tool to develop therapeutics). The specific microbiotic actionable targets starting with microbiotic strains obtained from, e.g. fecal matter transplants (FMT), the microorganism(s), the genus, and the presence/absence of microorganism strain(s) related to health conditions or diseases can be determined using the Complete Biome Test.

FIG. 4 (A) depicts the microbiome strain resolution using standard tests and (B) the increased microbiome strain resolution using the Complete Biome Test.

FIG. 5 depicts an illustrative process for generating a database using data obtained from the group consisting of: external data (e.g. scientific literature and/or databases), patient information, measured epigenetic changes, measured functional pathways, measured strain classification, and any combinations thereof. The database can be used, e.g. to drive identification of a therapeutic consortia (e.g. for treatment of health conditions or diseases).

FIG. 6 depicts how both the diagnostic and therapeutic approach outlined herein can comprise a targeted microbe strain selection as compared to a composite fecal microbiome transplant.

FIG. 7 depicts a system adapted to enable a user to detect, analyze, and process data (e.g. sequencing data, strain classification, functional pathways, epigenetic changes, patient information, external data, databases, microbiome strains; therapeutic consortia, etc.) using machine readable code.

FIG. 8 illustrates an example of high-throughput viability testing using a Start Growth Time (SGT) assay in 384-well format.

FIG. 9 illustrates an individual curve for SGT analysis. For example, time of growth (vertical dashed line) can be determined for reaching a certain OD (horizontal dashed line). Growth speed (e.g., doubling time) can be determined using slope (dotted line).

FIG. 10 illustrates SGT delta between different growth conditions. Growth curve lag between conditions can be compared to determine the relative percentage of live cells.

FIG. 11 illustrates quantitation of live cells. Time can be translated to Dilution value, which can be used to calculate percentage of live cells.

FIG. 12 illustrates an assay to compare percentage of live cells across various conditions. Absolute value and/or variance can be compared.

FIG. 13 illustrates an assay to quantitate live cells using, for example, a flow cytometer. Thiazole orange (TO) can stain live and dead cells green. Propidium iodide (PO) can stain dead cells red. Using a flow cytometer, live (e.g., right side in each panel) and dead cells (e.g., left side in each panel) can be counted. A ratio of live vs dead cell count (e.g., live/dead) can be used to create a standard curve for measurements. These results can be confirmed and/or compared to standard Colony Forming Unit (CFU) plate counting.

FIG. 14 illustrates an example of a correlation of live/dead cell ratio, determined from flow cytometer, with Optical Density (OD; e.g., OD₆₀₀) for the microbe B. longum. This can allow normalization of OD values to exact viable cell count. OD measurements, for example, during the log-phase growth, can then be used in addition to/or in lieu of standard Colony Forming Unit (CFU) plate counting, once this normalization has been performed for a particular strain.

### DETAILED DESCRIPTION OF THE INVENTION

### Definitions

As used in the specification and claims, the singular forms “a”, “an” and “the” include plural references unless the context clearly dictates otherwise. For example, the term “a sample” includes a plurality of samples, including mixtures thereof.

The terms “microbes” and “microorganisms” are used interchangeably herein and can refer to bacteria, archaea, eukaryotes (e.g. protozoa, fungi, yeast), and viruses, including bacterial viruses (i.e. phage).

The term “microbiome”, “microbiota”, and “microbial habitat” are used interchangeably herein and can refer to the ecological community of microorganisms that live on or in a subject's body. The microbiome can be comprised of commensal, symbiotic, and/or pathogenic microorganisms. Microbiomes can exist on or in many, if not most parts of the subject. Non-limiting examples of habitats of microbiome can include: body surfaces, body cavities, body fluids, the gut, the colon, skin, skin surfaces, skin pores, vaginal cavity, umbilical regions, conjunctival regions, intestinal regions, the stomach, the nasal cavities and passages, the gastrointestinal tract, the urogenital tracts, saliva, mucus, and feces.

The term “prebiotic” as used herein can be a general term to refer to chemicals and/or ingredients that can affect the growth and/or activity of microorganisms in a host. Prebiotics can allow for specific changes in the composition and/or activity in the microbiome. Prebiotics can confer a health benefit on the host. Prebiotics can be selectively fermented, e.g. in the colon. Non-limiting examples of prebiotics can include: complex carbohydrates, complex sugars, resistant dextrins, resistant starch, amino acids, peptides, nutritional compounds, biotin, polydextrose, oligosaccharides, polysaccharide, fructooligosaccharide (FOS), fructans, soluble fiber, insoluble fiber, fiber, starch, galactooligosaccharides (GOS), inulin, lignin, psyllium, chitin, chitosan, gums (e.g. guar gum), high amylose cornstarch (HAS), cellulose, β-glucans, hemi-celluloses, lactulose, mannooligosaccharides, mannan oligosaccharides (MOS), oligofructose-enriched inulin, oligofructose, oligodextrose, tagatose, trans-galactooligosaccharide, pectin, resistant starch, and xylooligosaccharides (XOS). Prebiotics can be found in foods, for example, acacia gum, guar seeds, brown rice, rice bran, barley hulls, chicory root, Jerusalem artichoke, dandelion greens, garlic, leek, onion, asparagus, wheat bran, oat bran, baked beans, whole wheat flour, and banana. Prebiotics can be found in breast milk. Prebiotics can be administered in any suitable form, for example, capsule and dietary supplement.

The term “probiotic” as used herein can mean one or more microorganisms which, when administered appropriately, can confer a health benefit on the host or subject. Non-limiting examples of probiotics include, for example, Akkermansia muciniphila, Anaerostipes caccae, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium coccoides, Clostridium indolis, Clostridium nexile, Clostridium orbiscindens, Clostridium propionicum, Clostridium xylanolyticum, Enterococcus faecium, Eubacterium hallii, Eubacterium rectale, Faecalibacterium prausnitzii, Fibrobacter succinogenes, Lactobacillus acidophilus, Lactobacillus brevis, Lactobacillus bulgaricus, Lactobacillus casei, Lactobacillus caucasicus, Lactobacillus fermentum, Lactobacillus helveticus, Lactobacillus lactis, Lactobacillus plantarum, Lactobacillus reuteri, Lactobacillus rhamnosus, Oscillospira guilliermondii, Roseburia cecicola, Roseburia inulinivorans, Ruminococcus flavefaciens, Ruminococcus gnavus, Ruminococcus obeum, Stenotrophomonas nitritireducens, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus, Coprococcus eutactus, Eubacterium cylindroides, Eubacterium dolichum, Eubacterium ventriosum, Roseburia faeccis, Roseburia hominis, Roseburia intestinalis, Lacatobacillus bifidus, Lactobacillus johnsonii, Lactobacilli, Akkermansia, Bifidobacteria, Clostridia, Eubacteria, Verrucomicrobia, and Firmicutes.

The terms “determining”, “measuring”, “evaluating”, “assessing,” “assaying,” and “analyzing” can be used interchangeably herein and can refer to any form of measurement, and include determining if an element is present or not (e.g., detection). These terms can include both quantitative and/or qualitative determinations. Assessing may be relative or absolute. These terms can include use of the algorithms and databases described herein. “Detecting the presence of” can include determining the amount of something present, as well as determining whether it is present or absent. The term “genome assembly algorithm” as used herein, refers to any method capable of aligning sequencing reads with each other (de novo) or to a reference (re-sequencing) under conditions that a complete sequence of the genome may be determined.

The term “genome” as used herein, can refer to the entirety of an organism's hereditary information that is encoded in its primary DNA sequence. The genome includes both the genes and the non-coding sequences. For example, the genome may represent a microbial genome. The genetic content of the microbiome can comprise: genomic DNA, RNA, and ribosomal RNA, the epigenome, plasmids, and all other types of genetic information found in the microbes that comprise the microbiome.

“Nucleic acid sequence” and “nucleotide sequence” as used herein refer to an oligonucleotide or polynucleotide, and fragments or portions thereof, and to DNA or RNA of genomic or synthetic origin which may be single- or double-stranded, and represent the sense or antisense strand. The nucleic acid sequence can be made up of adenine, guanine, cytosine, thymine, and uracil (A, T, C, G, and U) as well as modified versions (e.g. N6-methyladenosine, 5-methylcytosine, etc.).

The terms “homology” and “homologous” as used herein in reference to nucleotide sequences refer to a degree of complementarity with other nucleotide sequences. There may be partial homology or complete homology (i.e., identity). A nucleotide sequence which is partially complementary, i.e., “substantially homologous,” to a nucleic acid sequence is one that at least partially inhibits a completely complementary sequence from hybridizing to a target nucleic acid sequence.

The term “sequencing” as used herein refers to sequencing methods for determining the order of the nucleotide bases—A, T, C, G, and U—in a nucleic acid molecule (e.g., a DNA or RNA nucleic acid molecule.

The term “biochip” or “array” can refer to a solid substrate having a generally planar surface to which an adsorbent is attached. A surface of the biochip can comprise a plurality of addressable locations, each of which location may have the adsorbent bound there. Biochips can be adapted to engage a probe interface, and therefore, function as probes. Protein biochips are adapted for the capture of polypeptides and can be comprise surfaces having chromatographic or biospecific adsorbents attached thereto at addressable locations. Microarray chips are generally used for DNA and RNA gene expression detection.

The term “barcode” as used herein, refers to any unique, non-naturally occurring, nucleic acid sequence that may be used to identify the originating genome of a nucleic acid fragment.

The terms “subject,” “individual,” “host,” and “patient” can be used interchangeably herein and refer to any animal subject, including: humans, laboratory animals, livestock, and household pets. The subject can host a variety of microorganisms. The subject can have different microbiomes in various habitats on and in their body. The subject may be diagnosed or suspected of being at high risk for a disease. The subject may have a microbiome state that is contributing to a disease (i.e. dysbiosis). In some cases, the subject is not necessarily diagnosed or suspected of being at high risk for the disease. In some instances a subject may be suffering from an infection or at risk of developing or transmitting to others an infection.

The terms “treatment” or “treating” are used interchangeably herein. These terms can refer to an approach for obtaining beneficial or desired results including but not limited to a therapeutic benefit and/or a prophylactic benefit. A therapeutic benefit can mean eradication or amelioration of the underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The terms “16S”, “16S ribosomal subunit”, and “16S ribosomal RNA (rRNA)” can be used interchangeably herein and can refer to a component of a small subunit (e.g., 30S) of a prokaryotic (e.g., bacteria, archaea) ribosome. The 16S rRNA is highly conserved evolutionarily among species of microorganisms. Consequently, sequencing of the 16S ribosomal subunit can be used to identify and/or compare microorganisms present in a sample (e.g., a microbiome).

The terms “23S”, “23 S ribosomal subunit”, and “23 S ribosomal RNA (rRNA)” can be used interchangeably herein and can refer to a component of a large subunit (e.g., 50S) of a prokaryotic (e.g., bacteria, archaea) ribosome. Sequencing of the 23S ribosomal subunit can be used to identify and/or compare microorganisms present in a sample (e.g., a microbiome).

The term “spore” as used herein can refer to a viable cell produced by a microorganism to resist unfavorable conditions such as high temperatures, humidity, and chemical agents. A spore can have thick walls that allow the microorganism to survive harsh conditions for extended periods of time. Under suitable environmental conditions, a spore can germinate to produce a living form of the microorganism that is capable of reproduction and all of the physiological activities of the microorganism.

### Overview

Compositions comprising microbes can confer a variety of beneficial effects on a subject. Examples of these beneficial effects can include immunomodulatory features, regulation of cell proliferation, the ability to promote normal physiologic development of the mucosal epithelium, and enhancement of human nutrition. Microbial-based compositions can be administered as a therapeutic to a subject suffering from a microbiome-related health condition or disorder.

The invention disclosed herein provides compositions and methods for treating a skin disorder in a subject. In some embodiments, the invention disclosed herein provides compositions and methods for treating atopic dermatitis in a subject. In some embodiments, the invention disclosed herein provides compositions and methods for treating pediatric atopic dermatitis in a subject. In some embodiments, the treatment comprises modulating the disorder-associated microbiome, for example, skin and/or gut microbiome, of the subject. In some embodiments, the treatment comprises restoring the disorder-associated microbiome of the subject to a healthy state. In some embodiments, the treatment comprises modulating the pH of the skin of the subject. In some embodiments, the treatment comprises restoring the pH of the skin of the subject to a normal pH.

The disclosure provides methods and compositions to modulate the pH of a subject. In some embodiments, the disclosure provides methods and compositions to decrease the pH of a subject. In some embodiments, the disclosure provides methods and compositions to increase the pH of a subject. pH of any suitable part, surface, or fluid of the subject's body can be modulated such as increased, or decreased. Non-limiting examples of subject's body parts include: body surfaces, body cavities, body fluids, the gut, the colon, skin, skin surfaces, skin pores, vaginal cavity, umbilical regions, conjunctival regions, intestinal regions, the stomach, the nasal cavities and passages, the gastrointestinal tract, the urogenital tracts, saliva, mucus, and mucus membranes.

The disclosure provides methods and compositions to modulate the pH of skin in a subject. In some embodiments, the disclosure provides methods and compositions to decrease the pH of the skin in a subject. In some embodiments, the disclosure provides methods and compositions to increase the pH of the skin in a subject.

The disclosure provides methods and compositions to modulate and/or restore (e.g., to a healthy state) one or more microbiomes of a subject. In some embodiments, the disclosure provides methods and compositions to modulate and/or restore the skin microbiome of a subject. In some embodiments, the disclosure provides methods and compositions to modulate and/or restore the gut microbiome of a subject. In some embodiments, the disclosure provides methods and compositions to modulate and/or restore skin and gut microbiome of a subject.

In some embodiments, the disclosure provides a diagnostic test to predict the likelihood or determine the status of a skin disorder in a subject. The diagnostic test can use the presence of one or more microbes, altered microbiome, and pH changes. The diagnostic test can use personal characteristics, for example, age, weight, gender, medical history, risk factors, family history, or a combination thereof. The diagnostic assay can further use environmental factors such as geographic location, type of work, and use of hygiene products.

### Skin Microbiome

The skin is the largest organ of the human body and can be colonized by numerous microorganisms. The microorganism can be commensal or symbionts. Non-limiting examples of microbes that can be a part of the skin microbiome include species of micrococcus, staphylococcus such as Staphylococcus epidermidis and Staphylococcus hominis, bacteriodetes, corynebacterium, brevibacterium, dermabacter, malasezzia, acinetobacter, propionibacteria, demodex such as Demodex folliculorum and Demodex brevis, malasezzia, proteobacteria, actinobacteria, firmicutes, and cyanobacteria.

The composition can comprise one or more resident microbes of a microbiome habitat (e.g., resident skin microbiome). The composition can comprise, for example, micrococcus, staphylococcus, Staphylococcus epidermidis, Staphylococcus hominis, bacteriodetes, corynebacterium, brevibacterium, dermabacter, malasezzia, acinetobacter, propionibacteria, demodex such as Demodex folliculorum and Demodex brevis, malasezzia, proteobacteria, actinobacteria, firmicutes, and cyanobacteria. The microbes can be present in a pure and/or isolated form.

The microbiome of healthy skin can be composed primarily of Staphylococcus epidermidis (S. epidermidis). S. epidermidis can contribute to maintenance of the skin defense system. S. epidermidis can secrete various factors to inhibit colonization and growth of other microorganisms.

S. epidermidis can secrete antimicrobial peptides such as phenol-soluble modulin (PSM), which can prevent other bacterial strains from growing. Non-limiting examples of PSMs include PSM alpha, PSM alpha 3, PSM beta, PSM gamma, and PSM delta. These antimicrobial peptides can inhibit other microbes, for example, Staphylococcus aureus, Group A Streptococcus, and E. coli, from colonizing and propagating on the skin, without affecting growth of the resident skin microbiome.

In some embodiments, the composition comprises one or more antimicrobial peptides. The antimicrobial peptide can be present in a pure form. In some embodiments, one or more antimicrobial peptides can be a part of the formulation in their pure form without necessarily the presence of the producing strain. In some embodiments, the composition comprises one or more antimicrobial peptide-producing strains. In some embodiments, the composition comprises one or more antimicrobial peptides and one or more antimicrobial peptide-producing strains.

S. epidermidis can release bacteriocins to inhibit non-resident bacteria from colonizing and growing on healthy skin. Non-limiting examples of bacteriocins secreted by S. epidermidis include epidermin, epilancin K7, epilancin 15×, pep5, and staphylococcin 1580.

In some embodiments, the composition comprises one or more bacteriocins. The bacteriocins can be present in a pure form. In some embodiments, one or more bacteriocins can be a part of the formulation in their pure form without necessarily the presence of the producing strain. In some embodiments, the composition comprises one or more bacteriocin-producing strains. In some embodiments, the composition comprises one or more bacteriocins and one or more bacteriocin-producing strains.

S. epidermidis can secrete enzymes to inhibit adherence and biofilm formation. For example, S. epidermidis strain JK16 can secrete the enzyme serine protease Esp. This enzyme can inhibit biofilm production and colonization by S. aureus.

In some embodiments, the composition comprises one or more enzymes, e.g., serine protease. In some embodiments, the composition comprises one or more adherence prevention and/or biofilm prevention enzymes. The adherence prevention and/or biofilm prevention enzymes can be present in a pure form. In some embodiments one or more adherence prevention and/or biofilm prevention enzymes can be part of the formulation in their pure form without necessarily the presence of the producing strain. In some embodiments, the composition comprises one or more adherence and/or biofilm prevention enzyme-producing strains. In some embodiments, the composition comprises one or more adherence and/or biofilm prevention enzymes and one or more adherence and/or biofilm prevention enzymes-producing strains.

S. epidermidis can interact with the host cells by stimulating Toll-Like Receptors (TLRs). TLRs can include, for example, TLR1, TLR2, TLR3, TLR4, TLR5, TLR6, TLR7, TLR8, TLR9, TLR10, TLR11, TLR12, and TLR13. In some instances, S. epidermidis can interact with TLR2. This interaction can activate the cytokine and inflammatory pathways. Subjects with inflammatory skin disorders, for example, atopic dermatitis, can have a mutated form of S. epidermidis, which can trigger inappropriate downstream signaling. A composition can comprise a microorganism that prevents or reduces the likelihood of stimulation of, for example, a toll-like receptor associated with activation of cytokine and inflammatory pathways.

### Skin Disorders

Dysbiosis of the skin microbiome can be implicated in skin disorders. Dysbiosis of other microbial habitats, for example, gut microbiome, can be implicated in skin disorders.

Atopic dermatitis can refer to a chronic inflammatory skin disorder. Atopic dermatitis can be associated with scaly skin, itchy rashes, and lesions. Atopic dermatitis can also be referred to as atopic eczema or eczema. The hallmark of atopic dermatitis can be pruritus, which refers to an itching condition that leads to rashes. Any part of the skin can be affected by atopic dermatitis. Age can play a role in the localization of atopic dermatitis. For example, in infants, atopic dermatitis can affect cheeks, scalp, trunk, and extremities; in early childhood, atopic dermatitis can localize to flexural areas; and in adolescents and adults, atopic dermatitis can affect hands and feet.

Atopic dermatitis can be common in children. Atopic dermatitis can be referred to as pediatric atopic dermatitis when present in infants, toddlers, and children. In the United States, the prevalence of atopic dermatitis can be about 10% in children. Atopic dermatitis can have an early age of onset. The disease onset can occur before the ages of 1 and 5 years in about 65% and about 85% of affected children, respectively. The number of office visits to a doctor for children with atopic dermatitis is increasing.

Diagnosis of atopic dermatitis can involve the use of skin biopsies and/or laboratory testing to exclude other skin disorders. Atopic dermatitis can be treated and/or symptoms of atopic dermatitis can be controlled by, for example, bathing, moisturizing, topical medication, changes in diet, allergy treatments, maintenance of skin care to repair, maintenance of a healthy skin barrier, regular bathing in lukewarm water, frequent application of moisturizer, acid bath, and bleach bath. Bleach bath, used to treat and/or control symptoms of atopic dermatitis, can serve as an antibiotic treatment. Bleach bath can kill all of the bacteria on the skin. Acid bath can lower the pH of the skin. Infectious triggers and/or infection-related flares, for example, due to S. aureus infection, can be controlled by bleach bath with intranasal mupirocin. Topical medications, for example, antibiotics, anti-inflammatory medications, topical steroids, can be used in various gradations. The application process for topical medications can involve, for example, soaking in a bath, application of a topical steroid, application of a moisturizer (e,g, ointment such as petroleum jelly), wrapping moist gauze or cotton clothing dampened with warm water, covering wet layer with dry cotton clothing, and leaving on for 3-8 hours. Wet dressings can be used continuously for 24-72 hours or overnight for up to a week. Topical calcineurin inhibitors (TCIs) can be used as a therapy for moderate-to-severe atopic dermatitis. TCIs can inhibit T cell function. TCIs can have a different adverse effect profile compared with topical steroids.

A number of factors can contribute to the pathophysiology of skin disorders. These can include, for example, T helper cell disregulation, production of IgE, mast cell hyperactivity, susceptibility genes, environmental factors, defective skin barrier function, and immunologic responses. A family or personal history of atopy (i.e. a genetic tendency to develop allergic diseases), ichthyosis vulgaris, and xerosis can increase the risk of developing skin disorders. Other inflammatory skin conditions and disorders that can present similarly with atopic dermatitis include, for example, contact dermatitis, seborrheic dermatitis, and psoriasis. Patients with skin disorders can be at a greater risk of viral skin infections, for example, the potentially life threatening eczema herpeticum.

Food allergies can play a role in skin disorders. Common food allergies can be IgE-mediated and can consist of acute utricarial angioedema and contact reactions. Food allergies can increase symptoms of skin disorders. When symptoms of skin disorders, for example, atopic dermatitis, become worse, the mechanistic basis for inflammatiory reactions may not be IgE-mediated, but rather delayed hypersensitivity reactions that can develop 2-6 hours after exposure to the food. Prevalence of food allergy in all children in the first 5 years of life can be about 5%. Prevalence of food allergy in children with skin disorders, for example, atopic dermatitis, can be about 30-40%. Patients with food allergy can have an earlier onset and/or a more severe form of atopic dermatitis. Patients with early onset of skin disorders can have a higher risk of developing food allergies than those with later onset. The presence of food allergy can predict a poor prognosis of severe and persistent skin disorders. In patients with food sensitivity and/or allergy, food allergens can induce urticarial lesions, itching, and eczematous flares. These clinical phenotypes associated with food allergies can aggravate skin disorders.

Dysfunction of the skin barrier can play a role in skin disorders, for example, atopic dermatitis. Filaggrin is a filament-aggregating protein that can bind to keratin fibers in epithelial cells and contribute to the formation of stratum corneum barrier. Mutations in filaggrin can be associated with an increased risk, for example, two- to three-fold higher risk, of developing skin disorders, for example, atopic dermatitis. More than 40 loss of function filaggrin mutations have been identified and associated with up to 50% of patients with atopic dermatitis. Inadequate filaggrin production can reduce the ability of keratinocytes to maintain hydration and/or restrict transepidermal water loss, leading to pruritus and/or atopic dermatitis. Inadequate filaggrin production can increase the ability of aeroallergens to enter the body more easily. These aeroallergens can cause an inflammatory response, leading to skin disorders, for example, atopic dermatitis.

Changes in pH can play a role in skin disorders. Local pH increases can lead to an overgrowth of bacteria such as Staphylococcus aureus (S. aureus). This microbial overgrowth can trigger an innate immune response resulting in an inflammatory response that can lead to skin disorders.

Impairment in microbiome of any habitat of the subject can be associated with skin disorders. In some cases, the skin microbial habitat is impaired leading to a skin disorder. In some cases, the gut microbial habitat is impaired leading to a skin disorder. Non-limiting examples of microbial habitats that can be impaired and lead to skin disorders include, body surfaces, body cavities, body fluids, gut, colon, skin surfaces, skin pores, vaginal cavity, umbilical regions, conjunctival regions, intestinal regions, stomach, nasal cavities, nasal passages, gastrointestinal tract, urogenital tract, saliva, mucus, feces, and any combination thereof.

A correlation between skin disorders (e.g., atopic dermatitis) and bacterial vaginosis can be present. Bacterial vaginosis can refer to a microbial infection of the vagina. Bacterial vaginosis can be characterized by increased pH. Microbiome of a healthy vagina can be primarily composed of lactobacilli. Under normal conditions of low pH in the vagina, lactobacilli can produce hydrogen peroxide. Hydrogen peroxide can help limit the growth of other microorganisms in the vagina to healthy levels in two ways. Firstly, hydrogen peroxide can produce toxic hydroxyl radicals. Secondly, hydrogen peroxide can combine with a heavy pool of chlorine ions present in the vagina to produce chloridinium ions. Under circumstances of increased alkalinity in the vagina, for example, bleeding in pregnancy, sexual intercourse, or vaginal douching, or under circumstances where antibiotics are used or where there is a change in endocrine status, lactobacilli at high pH can be less efficient at producing hydrogen peroxide. This reduction in hydrogen peroxide can permit overgrowth of other organisms, leading to a change in the vaginal microbiome, and infections, for example, bacterial vaginosis. A diagnostic test that utilized a combination of lactobacilli representation, hydrogen peroxide concentration, and pH level could be predictive of several health indications, for example, bacterial vaginosis and preterm delivery.

A correlation between skin disorder and metabolic disorder can be present. The gut microbiome can be strongly involved with training the immune system. Dysbiosis of the gut microbiome can lead to many downstream inflammation-based reactions that can manifest in several ways, including on the skin as with atopic dermatitis. Correcting the gut dysbiosis can alleviate downstream inflammation of the skin. Method for diagnosis and treatment of gut dysbiosis are described in PCT/US2015/58511, which is herein incorporated by reference in its entirety for all purposes.

Low butyrate levels can be implicated in skin disorders such as atopic dermatitis. In some embodiments, the composition comprises one or more butyrate-producing microbes. In some embodiments, the composition comprises a microbe that encodes for an enzyme selected from the group consisting of: butyrate kinase, butyrate coenzyme A, butyrate coenzyme a transferase, and any combination thereof. A formulation comprising a prebiotic (e.g. inulin), a primary fermenter (e.g. Bifidobacterium), and a secondary fermenter (e.g. Clostridium and/or Eubacterium) can be used for butyrate production.

Butyrate can be an anti-inflammatory factor. Butyrate can affect gut permeability. Low levels of butyrate producing bacteria (e.g. Clostridium clusters XIVa and IV) and/or reduced lactate producing bacteria (e.g. Bifidobacterium adolescentis) can be correlated with, for example, skin disorders, gut dysbiosis, and metabolic disorders. Subsets of a formulation that comprise at least one primary fermenter and at least one secondary fermenter can be used for the treatment and/or mitigate progression of a skin condition.

In the colon, dietary fiber can be processed by butyrate-producing microorganisms to produce butyrate (i.e. butanoate), which is a short chain fatty acid (SCFA). In turn, butyrate can initiate G-protein coupled receptor (GPCR) signaling, leading to, for example, glucagon-like peptide-1 (GLP-1) secretion. GLP-1 can result in increased insulin sensitivity. Alteration of butyrate-producing microbiome in a subject can be associated with a skin disorder.

Butyrate kinase is an enzyme that can belong to a family of transferases, for example those transferring phosphorus-containing groups (e.g., phosphotransferases) with a carboxy group as acceptor. The systematic name of this enzyme class can be ATP:butanoate 1-phosphotransferase. Butyrate kinase can participate in butyrate metabolism. Butyrate kinase can catalyze the following reaction:

ADP+butyryl-phosphate⇄ATP+butyrate

Butyrate-Coenzyme A, also butyryl-coenzyme A, can be a coenzyme A-activated form of butyric acid. It can be acted upon by butyryl-CoA dehydrogenase and can be an intermediary compound in acetone-butanol-ethanol fermentation. Butyrate-Coenzyme A can be involved in butyrate metabolism.

Butyrate-Coenzyme A transferase, also known as butyrate-acetoacetate CoA-transferase, can belong to a family of transferases, for example, the CoA-transferases. The systematic name of this enzyme class can be butanoyl-CoA:acetoacetate CoA-transferase. Other names in common use can include butyryl coenzyme A-acetoacetate coenzyme A-transferase, and butyryl-CoA-acetoacetate CoA-transferase. Butyrate-Coenzyme A transferase can catalyze the following chemical reaction:

butanoyl-CoA+acetoacetate⇄butanoate+acetoacetyl-CoA

Butyryl-CoA dehydrogenase can belong to the family of oxidoreductases, for example, those acting on the CH—CH group of donor with other acceptors. The systematic name of this enzyme class can be butanoyl-CoA:acceptor 2,3-oxidoreductase. Other names in common use can include butyryl dehydrogenase, unsaturated acyl-CoA reductase, ethylene reductase, enoyl-coenzyme A reductase, unsaturated acyl coenzyme A reductase, butyryl coenzyme A dehydrogenase, short-chain acyl CoA dehydrogenase, short-chain acyl-coenzyme A dehydrogenase, 3-hydroxyacyl CoA reductase, and butanoyl-CoA:(acceptor) 2,3-oxidoreductase. Non-limiting examples of metabolic pathways that butyryl-CoA dehydrogenase can participate in include: fatty acid metabolism; valine, leucine and isoleucine degradation; and butanoate metabolism. Butyryl-CoA dehydrogenase can employ one cofactor, FAD. Butyryl-CoA dehydrogenase can catalyze the following reaction:

butyryl-CoA+acceptor ⇄2-butenoyl-CoA+reduced acceptor

Beta-hydroxybutyryl-CoA dehydrogenase or 3-hydroxybutyryl-CoA dehydrogenase can belong to a family of oxidoreductases, for example, those acting on the CH—OH group of donor with NAD+ or NADP+ as acceptor. The systematic name of the enzyme class can be (S)-3-hydroxybutanoyl-CoA:NADP+ oxidoreductase. Other names in common use can include beta-hydroxybutyryl coenzyme A dehydrogenase, L(+)-3-hydroxybutyryl-CoA dehydrogenase, BHBD, dehydrogenase, L-3-hydroxybutyryl coenzyme A (nicotinamide adenine, dinucleotide phosphate), L-(+)-3-hydroxybutyryl-CoA dehydrogenase, and 3-hydroxybutyryl-CoA dehydrogenase. Beta-hydroxybutyryl-CoA dehydrogenase enzyme can participate in benzoate degradation via coa ligation. Beta-hydroxybutyryl-CoA dehydrogenase enzyme can participate in butanoate metabolism. Beta-hydroxybutyryl-CoA dehydrogenase can catalyze the following reaction:

(S)-3-hydroxybutanoyl-CoA+NADP⁺⇄3-acetoacetyl-CoA+NADPH+H⁺

Crotonase can comprise enzymes with, for example, dehalogenase, hydratase, isomerase activities. Crotonase can be implicated in carbon-carbon bond formation, cleavage, and hydrolysis of thioesters. Enzymes in the crotonase superfamily can include, for example, enoyl-CoA hydratase which can catalyse the hydratation of 2-trans-enoyl-CoA into 3-hydroxyacyl-CoA; 3-2trans-enoyl-CoA isomerase or dodecenoyl-CoA isomerise (e.g., EC 5.3.3.8), which can shift the 3-double bond of the intermediates of unsaturated fatty acid oxidation to the 2-trans position; 3-hydroxbutyryl-CoA dehydratase (e.g., crotonase; EC 4.2.1.55), which can be involved in the butyrate/butanol-producing pathway; 4-Chlorobenzoyl-CoA dehalogenase (e.g., EC 3.8.1.6) which can catalyze the conversion of 4-chlorobenzoate-CoA to 4-hydroxybenzoate-CoA; dienoyl-CoA isomerase, which can catalyze the isomerisation of 3-trans,5-cis-dienoyl-CoA to 2-trans,4-trans-dienoyl-CoA; naphthoate synthase (e.g., MenB, or DHNA synthetase; EC 4.1.3.36), which can be involved in the biosynthesis of menaquinone (e.g., vitamin K2); carnitine racemase (e.g., gene caiD), which can catalyze the reversible conversion of crotonobetaine to L-carnitine in Escherichia coli; Methylmalonyl CoA decarboxylase (e.g., MMCD; EC 4.1.1.41); carboxymethylproline synthase (e.g., CarB), which can be involved in carbapenem biosynthesis; 6-oxo camphor hydrolase, which can catalyze the desymmetrization of bicyclic beta-diketones to optically active keto acids; the alpha subunit of fatty acid oxidation complex, a multi-enzyme complex that can catalyze the last three reactions in the fatty acid beta-oxidation cycle; and AUH protein, which can be a bifunctional RNA-binding homologue of enoyl-CoA hydratase.

Thiolases, also known as acetyl-coenzyme A acetyltransferases (ACAT), can convert two units of acetyl-CoA to acetoacetyl CoA, for example, in the mevalonate pathway. Thiolases can include, for example, degradative thiolases (e.g., EC 2.3.1.16) and biosynthetic thiolases (e.g., EC 2.3.1.9). 3-ketoacyl-CoA thiolase, also called thiolase I, can be involved in degradative pathways such as fatty acid beta-oxidation. Acetoacetyl-CoA thiolase, also called thiolase II, can be specific for the thiolysis of acetoacetyl-CoA and can be involved in biosynthetic pathways such as poly beta-hydroxybutyric acid synthesis or steroid biogenesis. A thiolase can catalyze the following reaction:

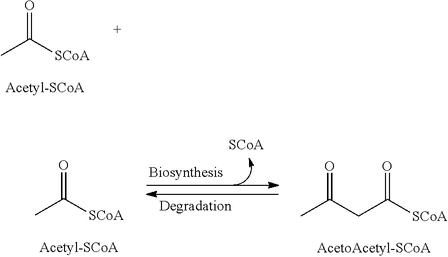

Production of butyrate can involve two major phases or microbes, for example, a primary fermenter and a secondary fermenter. The primary fermenter can produce intermediate molecules (e.g. lactate, acetate) when given an energy source (e.g. fiber). The secondary fermenter can convert the intermediate molecules produced by the primary fermenter into butyrate. Non-limiting examples of primary fermenter include Akkermansia muciniphila, Bifidobacterium adolescentis, Bifidobacterium infantis and Bifidobacterium longum. Non-limiting examples of secondary fermenter include Clostridium beijerinckii, Clostridium butyricum, Clostridium indolis, Eubacterium hallii, and Faecalibacterium prausnitzii. A combination of primary and secondary fermenters can be used to produce butyrate in a subject. Subsets of a formulation that comprises at least one primary fermenter and at least one secondary fermenter can be used for the treatment and/or mitigate progression of a metabolic health condition, for example, skin disorders. The formulation can additionally comprise a prebiotic.

In some embodiments, a therapeutic composition comprises at least one primary fermenter and at least one secondary fermenter. In some embodiments, a therapeutic composition comprises at least one primary fermenter, at least one secondary fermenter, and at least one prebiotic. In one non-limiting example, a therapeutic composition can comprise Bifidobacterium adolescentis, Clostridium indolis, and inulin. In another non-limiting example, a therapeutic composition can comprise Bifidobacterium longum, Faecalibacterium prausnitzii, and starch.

Akkermansia mucimphila can be a gram negative, strict anaerobe that can play a role in mucin degradation. Akkermansia mucimphila can be associated with increased levels of endocannabinoids that control inflammation, the gut barrier, and gut peptide secretion. Akkermansia mucimphila can serve as a primary fermenter.

Bifidobacterium adolescentis can be a gram-positive anaerobe, which can be found in healthy human gut from infancy. Bifidobacterium adolescentis can synthesize B vitamins. Bifidobacterium adolescentis can serve as a primary fermenter.

Bifidobacterium infantis can be a gram-positive, catalase negative, micro-aerotolerant anaerobe. Bifidobacterium infantis can serve as a primary fermenter.

Bifidobacterium longum can be a gram-positive, catalase negative, micro-aerotolerant anaerobe. Bifidobacterium longum can serve as a primary fermenter.

Clostridium beijerinckii can be a gram-positive, strict anaerobe that belongs to Clostridial cluster I. Clostridium beijerinckii can serve as a secondary fermenter.

Clostridium butyricum can be a gram-positive, strict anaerobe that can serve as a secondary fermenter.

Clostridium indolis can be a gram-positive, strict anaerobe that belongs to Clostridial cluster XIVA. Clostridium indolis can serve as a secondary fermenter.

Eubacterium hallii can be a gram-positive, anaerobe that belongs to Arrangement A Clostridial cluster XIVA. Eubacterium hallii can serve as a secondary fermenter.

Faecalibacterium prausnitzii can be a gram-positive, anaerobe belonging to Clostridial cluster IV. Faecalibacterium prausnitzii can be one of the most common gut bacteria and the largest butyrate producer. Faecalibacterium prausnitzii can serve as a secondary fermenter.

Non-limiting examples of genes and/or proteins involved in the generation of butyrate include: butyryl-CoA dehydrogenase, beta-hydroxybutyryl-CoA dehydrogenase or 3-hydroxybutyryl-CoA dehydrogenase, crotonase, electron transfer protein a, electron transfer protein b, and thiolase.

### Methods for Determining a Microbial Habitat

The present disclosure provides methods and compositions comprising microbial populations for the treatment of microbiome-related health conditions and/or disorders in a subject. Methods of the disclosure can include collection, stabilization and extraction of microbes for microbiome analysis. Methods of the disclosure can include determining the microbiome profile of any suitable microbial habitat of the subject. The composition of the microbial habitat can be used to diagnose a health condition of a subject, for example, to determine likelihood of a skin disorder and/or treatment course of the disorder.

An exemplary method of the disclosure can comprise at least one of the following steps: obtaining a sample from a subject, measuring a panel of microbes in the sample, comparing the panel of microbes in the sample with microbes found in a healthy sample, determining status of a disease upon the measuring, generating a report that provides information of disease status upon the results of the determining, and administering microbial-based compositions of the disclosure to the subject for treating a skin disorder such as a microbiome-based disorder, or the presence or absence of a microbe.

Methods for profiling a microbiome are discussed in U.S. patent application Ser. No. 14/437,133, which is incorporated herein by reference in its entirety for all purposes.

Detection methods, for example, long read sequencing, can be used to profile a microbiome and/or identify microbiome biomarkers. Comparison of the microbiome profile of a subject with a skin disorder, for example, atopic dermatitis with that of a healthy subject, can provide insights into microbial involvement in skin health and disease. Comparison of skin microbiome profiles of a healthy subject and a subject with for example, acne, can provide insights into microbial involvement in skin health and disease.

Subjects with normal skin and subjects with a skin disorder, for example, atopic dermatitis, can be categorized based on differences in species of microbes present in their microbiome. Subjects with skin disorders can have, for example, reduced microbial diversity, higher levels of non-resident microbes, higher levels of pathogenic microbes, high prevalence of S. aureus, and a lower diversity of the phylum bacteriodetes. The levels of S. aureus can increase during a flare up of atopic dermatitis symptoms.

Microbiomes of other parts of the body of subjects with skin disorders can be analyzed to determine whether a correlation exists with the altered skin microbiome. Microbiomes from, for example, body cavities, body fluids, gut, colon, vaginal cavity, umbilical regions, conjunctival regions, intestinal regions, the stomach, the nasal cavities and passages, the gastrointestinal tract, the urogenital tracts, saliva, mucus, and feces, can be analyzed and compared with that of healthy subjects. An increased and/or decreased diversity of gut microbiome can be associated with skin disorders. Subjects with skin disorders can have a lower prevalence of butyrate-producing bacteria, for example, C. eutactus.

In some embodiments, methods of the disclosure are used to analyze microbial habitat of the skin.

In some embodiments, methods of the disclosure can be used to determine microbial habitat of the gut or gastrointestinal tract of a subject. The gut comprises a complex microbiome including multiple species of microbes that can contribute to vitamin production and absorption, metabolism of proteins and bile acids, fermentation of dietary carbohydrates, and prevention of pathogen overgrowth. The composition of microbes within the gut can be linked to functional metabolic pathways in a subject. Non-limiting examples of metabolic pathways linked to gut microbiota include, energy balance regulation, secretion of leptin, lipid synthesis, hepatic insulin sensitivity, modulation of intestinal environment, and appetite signaling. Modification (e.g., dysbiosis) of the gut microbiome can increase the risk for health conditions such as skin disorders, mental disorders, ulcerative colitis, colorectal cancer, autoimmune disorders, obesity, diabetes, and inflammatory bowel disease.

In some embodiments, methods of the disclosure are used to analyze microbial habitat of the skin and gut. In some embodiments, methods of the disclosure are used to analyze microbial habitat of the gut.

In some embodiments, detection methods (e.g. sequencing) can be used to identify microbiome biomarkers associated with, for example, skin disorders such as atopic dermatitis.

In some embodiments, detection methods of the disclosure (e.g., sequencing) can be used to analyze changes in microbiome composition over time, for example, during antibiotic treatment, microbiome therapies, and various diets. The microbiome can be significantly altered upon exposure to antibiotics and diets that deplete the native microbial population. Methods of the disclosure can be used to generate profiles of the subject before and after administration of a therapeutic to characterize differences in the microbiota.

In some embodiments, methods to visualize the microbiome based on sequencing signatures are provided. In some embodiments, methods are provided to visualize the microbiome over time based on sequencing information.

Methods of the disclosure can be used to detect, characterize and quantify microbial habitat of a subject. The microbial habit can be used to define the diversity and abundance of microbes in order to evaluate clinical significance and causal framework for a disorder. Microbiome profiles can be compared to determine microbes that can be used as biomarkers for predicting and/or treating a health condition.

### Biological Samples

A biological sample can be collected from a subject to determine the microbiome profile of the subject. The biological sample can be any sample type from any microbial habitat on the body of a subject. Non-limiting examples of microbial habitats include skin habitat, umbilical habitat, vaginal habitat, amniotic fluid habitat, conjunctival habitat, intestinal habitat, stomach habitat, gut habitat, oral habitat, nasal habitat, gastrointestinal tract habitat, respiratory habitat, and urogenital tract habitat.

Depending on the application, the selection of a biological sample can be tailored to the specific application. The biological sample can be for example, whole blood, serum, plasma, mucosa, saliva, cheek swab, urine, stool, cells, tissue, bodily fluid, lymph fluid, CNS fluid, and lesion exudates. A combination of biological samples can be used with the methods of the disclosure.

In some embodiments, the sample is obtained from a subject's skin. Parts of the skin can be associated with their own unique microbiota. Non-limiting examples of skin (e.g., for sample collection) include cheeks, scalp, trunk, extremities, flexural areas, elbows, knees, neck, wrists, hands, eyelids, stomach, arms, chin, feet, glabella, alar crease, external auditory canal, nare, manubrium, axillary vault, antecubital fossa, volar forearm, hypothenar palm, interdigital web space, inguinal crease, umbilicus, toe web space, retroauricular crease, occiput, back, buttock, gluteal crease, popliteal foss, plantar heel, skin folds, cutaneous invaginations, appendages, sweat glands, eccrine glands, apocrine glands, sebaceous glands, hair follicles, stratum corneum, and sweat pore. Skin can include, for example, skin surface, epidermis, stratum corneum, stratum lucidem, stratum granulosum, stratum spinosum, stratum basale, dermis, connective tissue, hair follicles, hair shaft, sebaceous gland, sweat pore, and sweat glands

### Sample Preparation

Sample preparation can comprise any one of the following steps or a combination of steps. A sterile swab is first dipped into a tube containing sterile phosphate buffered saline (PBS) to wet. The swab is swiped across the area of interest multiple times (e.g., 10-20 times) with enough vigor that the tissue is slightly pink/red colored afterwards. The swab is gently dipped into a buffer (e.g., a lysis buffer) in a sterile tube. The swab is left in the tube for shipping to a laboratory to be further analyzed as provided herein. The samples obtained can be shipped overnight at room temperature. Shipping microbial cells in buffers can introduce detection bias in the samples. Some microbes can continue propagating on the nutrients that come along with sample collection. Some microbes can undergo apoptosis in the absence of a specific environment. As a result, microbial samples shipped in this fashion can have an initial profiling/population bias associated with cellular integrity.

Methods can be used to enrich intact cells by first centrifuging the collected sample. The resulting pellet, formed from the intact cells within the sample, can then be used as a precursor for all of the downstream steps. In some embodiments, the methods of the disclosure further comprise a purification step to concentrate any DNA present in the supernatant (e.g. from already lysed cells). This DNA can be combined with DNA extracted from the standard pellet preparation. The combined DNA can form a more complete precursor to the downstream steps.

Cell lysis and/or extraction of nucleic acids from the cells can be performed by any suitable methods including physical methods, chemical methods, or a combination of both. Nucleic acids can be isolated from a biological sample using shearing methods, which preserve the integrity and continuity of genomic DNA.

A nucleic acid sample used with the present disclosure can include all types of DNA and/or RNA. The length of nucleic acids can be about 100, 200, 300, 400, 500, 600, 700, 800, 900, 1000, 2000, 3000, 4000, 5000, 6000, 7000, 8000, 9000, 10,000, 20,000, 30,000, 40,000, 50,000, 60,000, 70,000, 80,000, 90,000, 100,000, 200,000, 300,000, 400,000, 500,000, 600,000, 700,000, 800,000, 900,000, 1,000,000, 2,000,000, 3,000,000, 4,000,000, 5,000,000, 6,000,000, 7,000,000, 8,000,000, 9,000,000, or 10,000,000, nucleotides or base pairs in length.

An amplicon approach can be used to prepare DNA for microbiome profiling. This approach can comprise a number of steps, for example, PCR, sample quantification (e.g. Qubit, nanodrop, bioanalyzer, etc.), Blue Pippin size selection, 0.5× Ampure purification, sample quantification, DNA end repair, 0.5× Ampure purification, blunt end adaptor ligation, exo-nuclease treatment, two 0.5× Ampure purifications, and final Blue Pippen size selection.

In some embodiments, the method does not use an amplification step. Examples of such methods include preparation of samples for sequencing by Whole Genome Shotgun (WGS) sequencing. These approaches can provide a benefit by removing amplification bias that can skew microbial distributions. In addition, such approaches can allow for de novo discovery of pertinent elements, for example, bacterial plasmids, fungi and viruses.

The practice of the methods of the present disclosure can employ conventional techniques of immunology, biochemistry, chemistry, molecular biology, microbiology, cell biology, genomics and recombinant DNA, which are within the skill of the art. For example, preparation of a biological sample can comprise, e.g., extraction or isolation of intracellular material from a cell or tissue such as the extraction of nucleic acids, protein, or other macromolecules. Sample preparation which can be used with the methods of disclosure include but are not limited to, centrifugation, affinity chromatography, magnetic separation, immunoassay, nucleic acid assay, receptor-based assay, cytometric assay, colorimetric assay, enzymatic assay, electrophoretic assay, electrochemical assay, spectroscopic assay, chromatographic assay, microscopic assay, topographic assay, calorimetric assay, radioisotope assay, protein synthesis assay, histological assay, culture assay, and combinations thereof.

### Microbiome Profiling

The present disclosure provides methods for measuring at least one microbe in a biological sample from at least one microbial habitat of a subject and determining a microbiome profile. A microbiome profile can be assessed using any suitable detection means that can measure or quantify one or more microbes (e.g., bacteria, fungi, viruses and archaea) that comprise a microbiome.

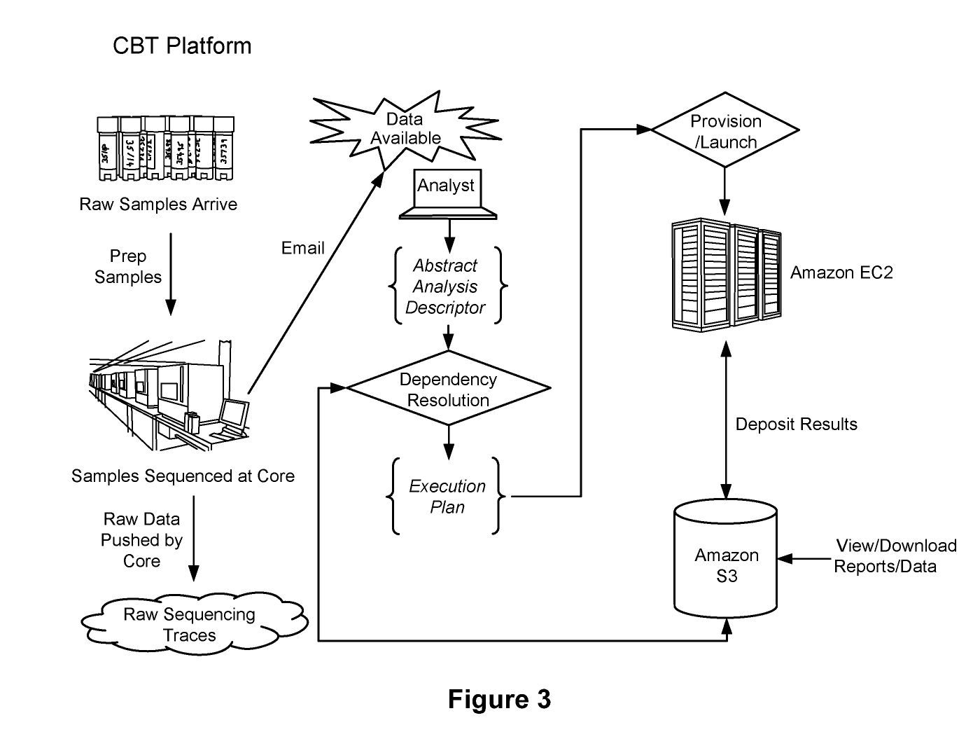

A Complete Biome Test (CBT) can generate microbiome profiles with, for example, strain-level resolution. A CBT can be performed using microbiome profiling methods described herein. FIG. 3 provides an illustration depicting an exemplary platform for a CBT (e.g. as a diagnostic test or as a development tool to develop therapeutics). The specific microbiotic actionable targets starting with microbiotic strains obtained from, e.g. fecal matter transplants (FMT), the microorganism(s), the genus, and the presence/absence of microorganism strain(s) related to health conditions or diseases can be determined using the CBT.

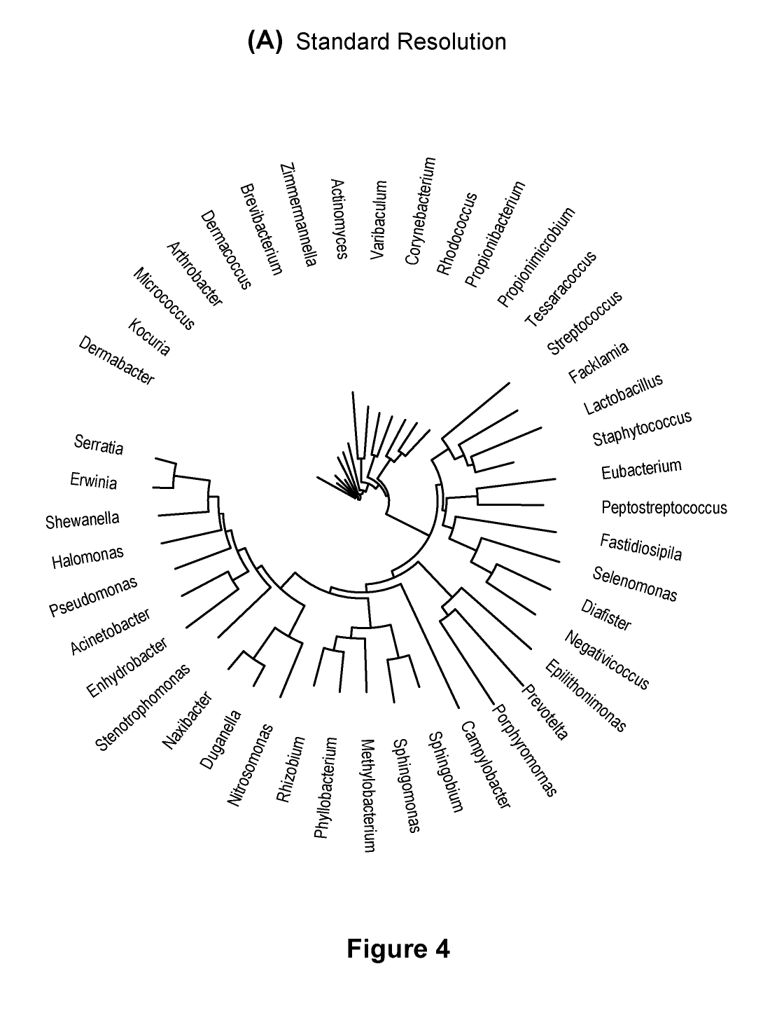

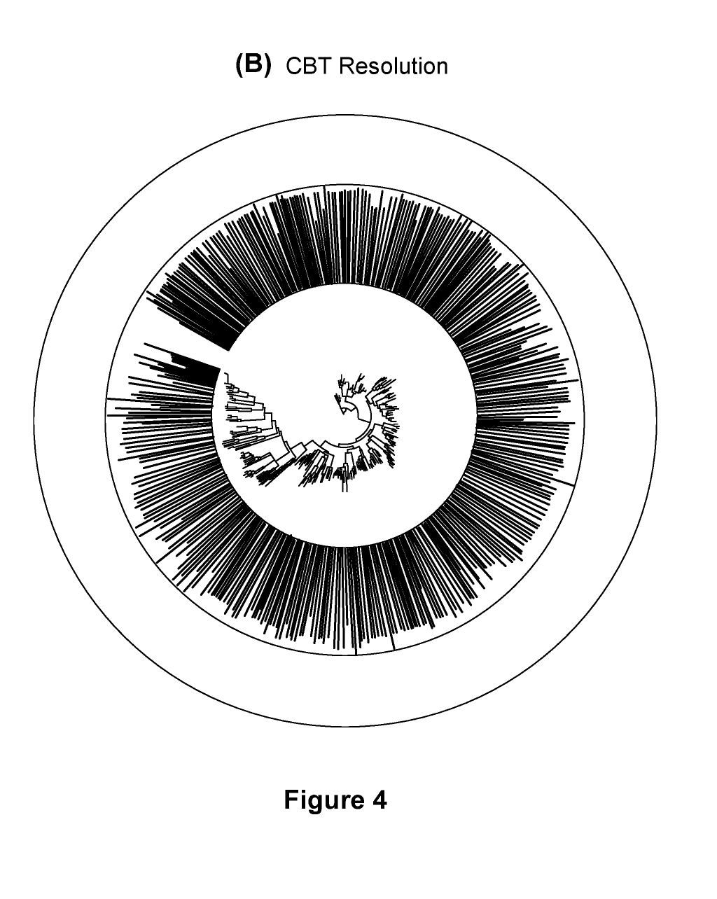

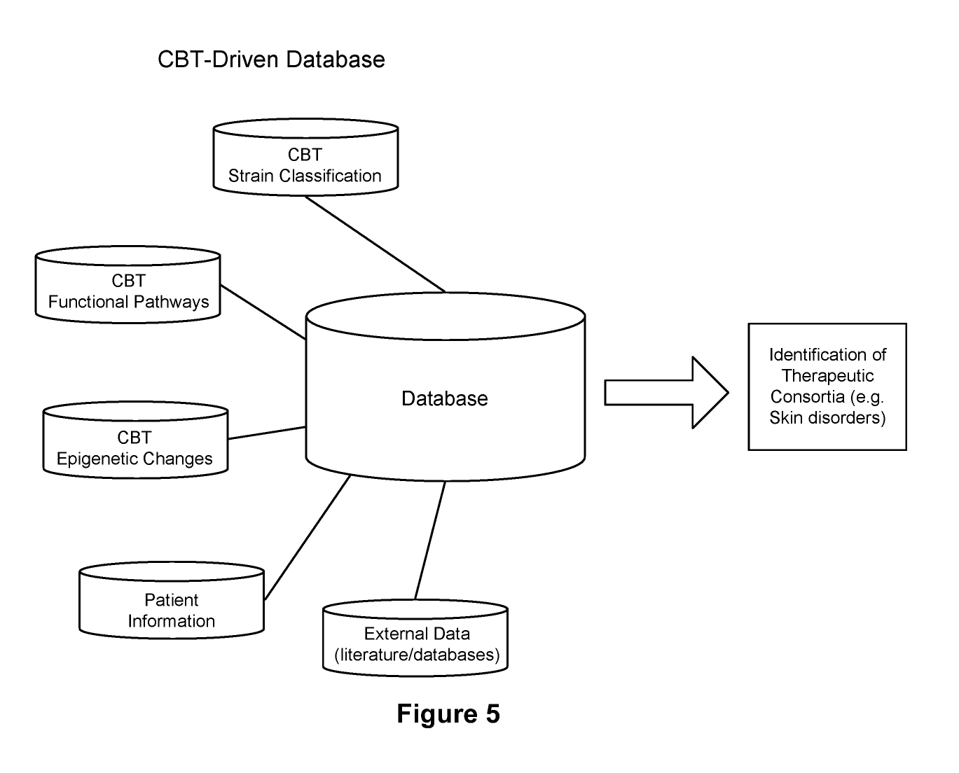

FIG. 4 (A) depicts the microbiome strain resolution using standard tests. FIG. 4 (B) depicts the increased microbiome strain resolution using the CBT. FIG. 5 depicts an illustrative process for generating a database (e.g., a CBT driven-database using data obtained from the group consisting of: external data (e.g. scientific literature and/or databases), patient information, measured epigenetic changes, measured functional pathways, measured strain classification, and any combinations thereof. The database can be used, e.g. to drive identification of a therapeutic consortia (e.g. for treatment of health conditions or diseases).

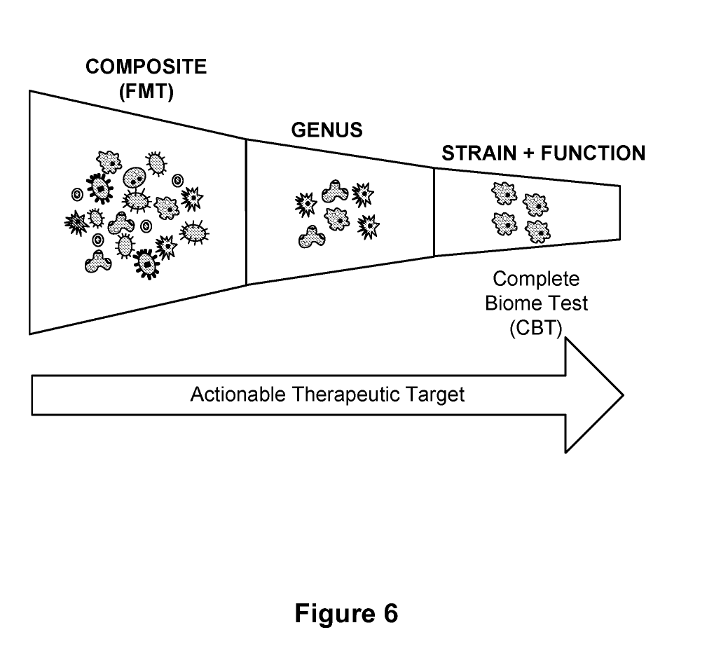

FIG. 6 depicts how both the diagnostic and therapeutic approach outlined herein can comprise a targeted microbe strain selection or therapeutic consortia as compared to a composite fecal microbiome transplant.

Nucleic acid sample prepared from a biological sample can be subjected to a detection method to generate a profile of the microbiome associated with the sample. Profiling of a microbiome can comprise one or more detection methods.

Methods of the disclosure can be used to measure, for example, a 16S ribosomal subunit, a 23S ribosomal subunit, intergenic regions, and other genetic elements. Suitable detection methods can be chosen to provide sufficient discriminative power in a particular microbe in order to identify informative microbiome profiles.

In some applications, a ribosomal RNA (rRNA) operon of a microbe is analyzed to determine a subject's microbiome profile. In some applications, the entire genomic region of the 16S or 23S ribosomal subunit of the microbe is analyzed to determine a subject's microbiome profile. In some applications, the variable regions of the 16S and/or 23S ribosomal subunit of the microbe are analyzed to determine a subject's microbiome profile.

In some applications, the entire genome of the microbe is analyzed to determine a subject's microbiome profile. In other applications, the variable regions of the microbe's genome are analyzed to determine a subject's microbiome profile. For example, genetic variation in the genome can include restriction fragment length polymorphisms, single nucleotide polymorphisms, insertions, deletions, indels (insertions-deletions), microsatellite repeats, minisatellite repeats, short tandem repeats, transposable elements, randomly amplified polymorphic DNA, amplification fragment length polymorphism or a combination thereof.

In some embodiments, sequencing methods such as long-read length single molecule sequencing is used for detection. Long read sequencing can provide microbial classification down to the strain resolution of each microbe. Examples of sequencing technologies that can be used with the present disclosure for achieving long read lengths include the SMRT sequencing systems from Pacific Biosciences, long read length Sanger sequencing, long read ensemble sequencing approaches, e.g., Illumina/Moleculo sequencing and potentially, other single molecule sequencing approaches, such as Nanopore sequencing technologies.

Long read sequencing can include sequencing that provides a contiguous sequence read of for example, longer than 500 bases, longer than 800 bases, longer than 1000 bases, longer than 1500 bases, longer than 2000 bases, longer than 3000 bases, or longer than 4500 bases.

In some embodiments, detection methods of the disclosure comprise ampification-mode sequencing to profile the microbiome. In some embodiments, detection methods of the disclosure comprise a non-amplification mode, for example, Whole Genome Shotgun (WGS) sequencing, to profile the microbiome.

Primers used in the disclosure can be prepared by any suitable method, for example, cloning of appropriate sequences and direct chemical synthesis. Primers can also be obtained from commercial sources. In addition, computer programs can be used to design primers. Primers can contain unique barcode identifiers.

Microbiome profiling can further comprise use of for example, a nucleic acid microarray, a biochip, a protein microarray, an analytical protein microarray, reverse phase protein microarray (RPA), a digital PCR device, and/or a droplet digital PCR device.

In some embodiments, the microbial profile is determined using additional information such as age, weight, gender, medical history, risk factors, family history, or any other clinically relevant information.

In some applications, a subject's microbiome profile comprises a single microbiome. For example, a subject's microbiome profile can comprise of at least one biological sample from only the subject's skin microbiome. In another example, a subject's microbiome profile can comprise of at least one biological sample from only the subject's intestinal microbiome. In another example, a subject's microbiome profile can comprise of at least one biological sample from only the subject's gut microbiome. In another example, a subject's microbiome profile can comprise of at least one biological sample from only the subject's oral microbiome.

In some applications, a subject's microbiome profile comprises at least one biological sample from more than one microbiome. For example, a subject's microbiome profile can comprise of at least one biological sample from the subject's skin microbiome and at least one biological sample from the gut microbiome. In another example, a subject's microbiome profile can comprise of at least one biological sample from the subject's skin microbiome, at least one biological sample from the intestinal microbiome, at least one biological sample from the gut microbiome, and at least one biological sample from the oral microbiome. In another example, a subject's microbiome profile can comprise of at least one biological sample from the subject's intestinal microbiome, and at least one biological sample from skin microbiome. In another example, a subject's microbiome profile can comprise of at least one biological sample from the subject's gut microbiome, and at least one biological sample from oral microbiome. In some applications, a subject's microbiome profile can comprise of at least 2, 3, 4, 5, 6, 7, 8, 9, 10, 11, 12, 13, 14, 15, 16, 17, 18, 19, 20 microbiomes.

A subject's microbiome profile can comprise of one microbe. In some applications, a subject's microbiome profile comprises of, for example, 2 microbes, 3 or fewer microbes, 4 or fewer microbes, 5 or fewer microbes, 6 or fewer microbes, 7 or fewer microbes, 8 or fewer microbes, 9 or fewer microbes, 10 or fewer microbes, 11 or fewer microbes, no more than 12 microbes, 13 or fewer microbes, 14 or fewer microbes, 15 or fewer microbes, 16 or fewer microbes, 18 or fewer microbes, 19 or fewer microbes, 20 or fewer microbes, 25 or fewer microbes, 30 or fewer microbes, 35 or fewer microbes, 40 or fewer microbes, 45 or fewer microbes, 50 or fewer microbes, 55 or fewer microbes, 60 or fewer microbes, 65 or fewer microbes, 70 or fewer microbes, 75 or fewer microbes, 80 or fewer microbes, 85 or fewer microbes, 90 or fewer microbes, 100 or fewer microbes, 200 or fewer microbes, 300 or fewer microbes, 400 or fewer microbe, 500 or fewer microbes, 600 or fewer microbes, 700 or fewer microbes, or 800 or fewer microbes.

### Algorithm-Based Methods

The present disclosure provides algorithm-based methods for building a microbiome profile of a subject. Non-limiting examples of algorithms that can be used with the disclosure include elastic networks, random forests, support vector machines, and logistic regression.

The algorithms can transform the underlying measurements into a quantitative score or probability relating to, for example, disease risk, disease likelihood, presence or absence of disease, presence or absence of a microbe, treatment response, and/or classification of disease status. The algorithms can aid in the selection of important microbes.

### Analysis

A microbiome profile of a subject can be analyzed to determine information related to the health status of the subject. The information can include, for example, degree of likelihood of a disorder, presence or absence of a disease state, a poor clinical outcome, good clinical outcome, high risk of disease, low risk of disease, complete response, partial response, stable disease, non-response, and recommended treatments for disease management.

The analysis can be a part of a diagnostic assay to predict disease status of a subject or likelihood of a subject's response to a therapeutic. The diagnostic assay can use the quantitative score calculated by the algorithms-based methods described herein to perform the analysis.

In some applications, an increase in one or more microbes' threshold values or quantitative score in a subject's microbiome profile indicates an increased likelihood of one or more of: a poor clinical outcome, good clinical outcome, high risk of disease, low risk of disease, complete response, partial response, stable disease, non-response, and recommended treatments for disease management. In some embodiments, a decrease in the quantitative score indicates an increased likelihood of one or more of: a poor clinical outcome, good clinical outcome, high risk of disease, low risk of disease, complete response, partial response, stable disease, non-response, and recommended treatments for disease management.

In some applications, a decrease in one or more microbes' threshold values or quantitative score in a subject's microbiome profile indicates a decreased likelihood of one or more of: a poor clinical outcome, good clinical outcome, high risk of disease, low risk of disease, complete response, partial response, stable disease, non-response, and recommended treatments for disease management. In some embodiments, a decrease in the quantitative score indicates an increased likelihood of one or more of: a poor clinical outcome, good clinical outcome, high risk of disease, low risk of disease, complete response, partial response, stable disease, non-response, and recommended treatments for disease management.

In some applications, an increase in one or more microbes' threshold values or quantitative score in a subject's microbiome profile indicates an increased likelihood of one or more of: a poor clinical outcome, good clinical outcome, high risk of disease, low risk of disease, complete response, partial response, stable disease, non-response, and recommended treatments for disease management. In some applications, a decrease in one or more microbes' threshold values indicates an increased likelihood of one or more of: a poor clinical outcome, good clinical outcome, high risk of disease, low risk of disease, complete response, partial response, stable disease, non-response, and recommended treatments for disease management.

In some applications, an increase in one or more microbes' threshold values or quantitative score in a subject's microbiome profile indicates a decreased likelihood of one or more of: a poor clinical outcome, good clinical outcome, high risk of disease, low risk of disease, complete response, partial response, stable disease, non-response, and recommended treatments for disease management. In some applications, a decrease in one or more microbes' threshold values indicates an increased likelihood of one or more of: a poor clinical outcome, good clinical outcome, high risk of disease, low risk of disease, complete response, partial response, stable disease, non-response, and recommended treatments for disease management.

In some applications, a similar microbiome profile to a reference profile indicates an increased likelihood of one or more of: a poor clinical outcome, good clinical outcome, high risk of disease, low risk of disease, complete response, partial response, stable disease, non-response, and recommended treatments for disease management. In some applications, a dissimilar microbiome profile to a reference profile indicates one or more of: an increased likelihood of a poor clinical outcome, good clinical outcome, high risk of disease, low risk of disease, complete response, partial response, stable disease, non-response, and recommended treatments for disease management.

In some applications, a similar microbiome profile to a reference profile indicates a decreased likelihood of one or more of: a poor clinical outcome, good clinical outcome, high risk of disease, low risk of disease, complete response, partial response, stable disease, non-response, and recommended treatments for disease management. In some applications, a dissimilar microbiome profile to a reference profile indicates one or more of: an increased likelihood of a poor clinical outcome, good clinical outcome, high risk of disease, low risk of disease, complete response, partial response, stable disease, non-response, and recommended treatments for disease management.

In some applications, a dissimilar microbiome profile to a reference profile indicates an increased likelihood of one or more of: a poor clinical outcome, good clinical outcome, high risk of disease, low risk of disease, complete response, partial response, stable disease, non-response, and recommended treatments for disease management. In some applications, a dissimilar microbiome profile to a reference profile indicates one or more of: an increased likelihood of a poor clinical outcome, good clinical outcome, high risk of disease, low risk of disease, complete response, partial response, stable disease, non-response, and recommended treatments for disease management.

In some applications, a dissimilar microbiome profile to a reference profile indicates a decreased likelihood of one or more of: a poor clinical outcome, good clinical outcome, high risk of disease, low risk of disease, complete response, partial response, stable disease, non-response, and recommended treatments for disease management. In some applications, a dissimilar microbiome profile to a reference profile indicates one or more of: an increased likelihood of a poor clinical outcome, good clinical outcome, high risk of disease, low risk of disease, complete response, partial response, stable disease, non-response, and recommended treatments for disease management.

### Accuracy and Sensitivity

The methods provided herein can provide strain classification of a genera, species or sub-strain level of one or more microbes in a sample with an accuracy of greater than 1%, 20%, 30%, 40%, 50%, 55%, 60%, 65%, 70%, 75%, 80%, 85%, 90%, 95%, 96%, 97%, 98%, 99%, 99.2%, 99.5%, 99.7%, or 99.9%. The methods provided herein can provide strain quantification of a genera, species or sub-strain level of one or more microbes in a sample with an accuracy of greater than 1%, 20%, 30%, 40%, 50%, 55%, 60%, 65%, 70%, 75%, 80%, 85%, 90%, 95%, 96%, 97%, 98%, 99%, 99.2%, 99.5%, 99.7%, or 99.9%.

The microbial profile can have an accuracy of 70% or greater based on measurement of 15 or fewer microbes in the biological sample. Such profiling method can have at least an accuracy greater than 70% based on measurement of no more than 2 microbes, 3 or fewer microbes, 4 or fewer microbes, 5 or fewer microbes, 6 or fewer microbes, 7 or fewer microbes, 8 or fewer microbes, 9 or fewer microbes, 10 or fewer microbes, 11 or fewer microbes, no more than 12 microbes, 13 or fewer microbes, 14 or fewer microbes, 15 or fewer microbes, 16 or fewer microbes, 18 or fewer microbes, 19 or fewer microbes, 20 or fewer microbes, 25 or fewer microbes, 30 or fewer microbes, 35 or fewer microbes, 40 or fewer microbes, 45 or fewer microbes, 50 or fewer microbes, 55 or fewer microbes, 60 or fewer microbes, 65 or fewer microbes, 70 or fewer microbes, 75 or fewer microbes, 80 or fewer microbes, 85 or fewer microbes, 90 or fewer microbes, or 100 or fewer microbes, 200 or fewer microbes, 300 or fewer microbes, 400 or fewer microbes, 500 or fewer microbes, 600 or fewer microbes, 700 or fewer microbes, or 800 or fewer microbes.

The diagnostic methods provided by the present disclosure for the diseases provided herein can have at least one of a sensitivity of 70% or greater and specificity of greater than 70% based on measurement of 15 or fewer microbes in the biological sample. Such diagnostic method can have at least one of a sensitivity greater than 70% and specificity greater than 70% based on measurement of no more than 2 microbes, 3 or fewer microbes, 4 or fewer microbes, 5 or fewer microbes, 6 or fewer microbes, 7 or fewer microbes, 8 or fewer microbes, 9 or fewer microbes, 10 or fewer microbes, 11 or fewer microbes, no more than 12 microbes, 13 or fewer microbes, 14 or fewer microbes, 15 or fewer microbes, 16 or fewer microbes, 18 or fewer microbes, 19 or fewer microbes, 20 or fewer microbes, 25 or fewer microbes, 30 or fewer microbes, 35 or fewer microbes, 40 or fewer microbes, 45 or fewer microbes, 50 or fewer microbes, 55 or fewer microbes, 60 or fewer microbes, 65 or fewer microbes, 70 or fewer microbes, 75 or fewer microbes, 80 or fewer microbes, 85 or fewer microbes, 90 or fewer microbes, or 100 or fewer microbes, 200 or fewer microbes, 300 or fewer microbes, 400 or fewer microbes, 500 or fewer microbes, 600 or fewer microbes, 700 or fewer microbes or 800 or fewer microbes.

The methods provided herein can provide a health status of a subject with a specificity greater than 1%, 20%, 30%, 40%, 50%, 55%, 60%, 65%, 70%, 75%, 80%, 85%, 90%, 95%, 96%, 97%, 98%, 99%, 99.2%, 99.5%, 99.7%, or 99.9% receiver operating characteristic (ROC). The methods provided herein can provide a health status of a subject with a sensitivity lesser than 1%, 20%, 30%, 40%, 50%, 55%, 60%, 65%, 70%, 75%, 80%, 85%, 90%, 95%, 96%, 97%, 98%, 99%, 99.2%, 99.5%, 99.7%, or 99.9% ROC.

### Methods for Treating a Subject

The disclosure provides methods for treating a subject. Altering the composition of a microbiome in a subject can have desired health consequences. Compositions of the disclosure can be administered as a therapeutic and/or a cosmetic for treating a health condition. Treatments designed to alter the host microbiome(s) can result in a reduction of patient symptoms, prevention of disease, and or treatment of the disease or health condition. For example, modification and/or restoration of the skin and/or gut microbiome can reduce the risk for health conditions such as skin disorders (e.g., atopic dermatitis).

The methods, compositions, and kits of the disclosure can comprise a method to treat, prevent, arrest, reverse, or ameliorate a disorder. In some embodiments, the disorder is a skin disorder. In some embodiments, the disorder is atopic dermatitis. In some embodiments, the modulation is achieved by administering a therapeutically-effective amount of a microbial-based composition at any body site that shows a correlated link to disease onset. In some embodiments, the composition is delivered to the skin of a subject. In some embodiments, the composition is delivered to the gut of a subject.

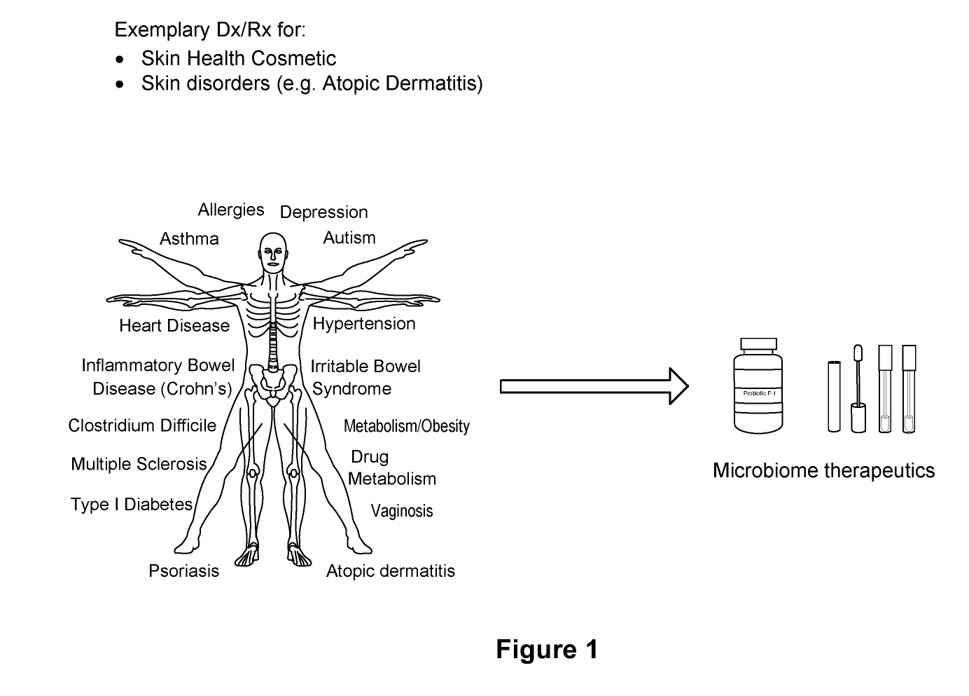

FIG. 1 depicts some non-limiting heath conditions that can be affected by the microbiome. These health conditions can include, for example, Type 2 Diabetes Mellitus (T2DM), preterm labor, chronic fatigue syndrome, skin conditions such as acne, allergies, autism, asthma, depression, hypertension, irritable bowel syndrome, metabolic syndrome, obesity, lactose intolerance, oral thrush, ulcerative colitis, drug metabolism, vaginosis, atopic dermatitis, psoriasis, Type I Diabetes Mellitus (T1DM), diabetes, Multiple Sclerosis, neurological disorders such as Parkinson's disease, Clostridium Difficile infection, Inflammatory Bowel Disease, Crohn's Disease, heart disease, diabetic foot ulcers, bacteremia, infantile colic, cancer, cystic fibrosis, multiple sclerosis, urinary tract infection, radiation enteropathy, drug metabolism, dental cavities, halitosis, metabolic disorder, gastrointestinal disorder, insulin insensitivity, metabolic syndrome, insulin defficiency, insulin resistance, glucose intolerance, Non Alcoholic Fatty Acid Liver Disease, Cardiovascular Disease, Hypertension, disorder associated with Cholesterol, and disorder associated with Triglycerides. The present disclosure can provide for a diagnostic assay of at least one microbiome that includes a report that gives guidance on health status or treatment modalities for the health conditions described herein. The present disclosure can also provide therapeutic and/or cosmetic formulations for treatment of health conditions described herein.

In some embodiments, the methods and compositions of the disclosure are used to treat a skin disorder. Non-limiting examples of skin conditions and disorders include acne, eczema, ichthyosis vulgaris, xerosis, pruritus, folliculitis, inflammation, allergy, bacterial infection, Staphylococcus aureus infection, fungal infection, psoriasis, rosacea, cold sores, plantar warts, palmer warts, hair loss, blisters, chafing, corns, calluses, sunburn, rashes, dermatitis, atopic dermatitis, pediatric atopic dermatitis, itching, hives, lesions, cysts, skin lumps, alopecia, vitiligo, lice, utricarial angioedema, scabies, bruise, epidermoid cyst, sebaceous cyst, cellulitis, leprosy, carbuncles, staph infection, impetigo, boils, pilonidal cyst, abscess, athlete's foot, jock itch, ringworm, candidiasis, molluscum contagiosum, sporotrichosis, seborrheic eczema, contact dermatitis, atopic eczema, seborrheic dermatitis, dyshidrotic eczema, nummular eczema, furunculosis, boils, abscesses, impetigo, school sores, methicillin-resistant Staph. Aureus, Staphylococcal scalded skin syndrome, toxic shock syndrome, tropical pyomyositis, botryomycosis, erysipelas, impetigo, necrotizing fasciitis, infectious gangrene, scarlet fever, rheumatic fever, and erythema marginatum.

In some embodiments, the skin disorder is dermatitis.

In some embodiments, the skin disorder is atopic dermatitis.

In some embodiments, the skin disorder is inflammation.

In some embodiments, the skin disorder is allergy.

The disclosure provides methods for the restoration of a microbial habitat of a subject to a healthy state. The method can comprise microbiome correction and/or adjustment including for example, replenishing native microbes, removing pathogenic microbes, administering prebiotics, and growth factors necessary for microbiome survival. In some embodiments, the method also comprises administering antimicrobial agents such as antibiotics.

Based on the microbiome profile, the present disclosure provides methods for generalized-treatment recommendation for a subject as well as methods for subject-specific treatment recommendation. Methods for treatments can comprise one of the following steps: determining a first ratio of a level of a subject-specific microbiome profile to a level of a second microbiome profile in a biological sample obtained from at least one subject, detecting a presence or absence of a disease in the subject based upon the determining, and recommending to the subject at least one generalized or subject-specific treatment to ameliorate disease symptoms.

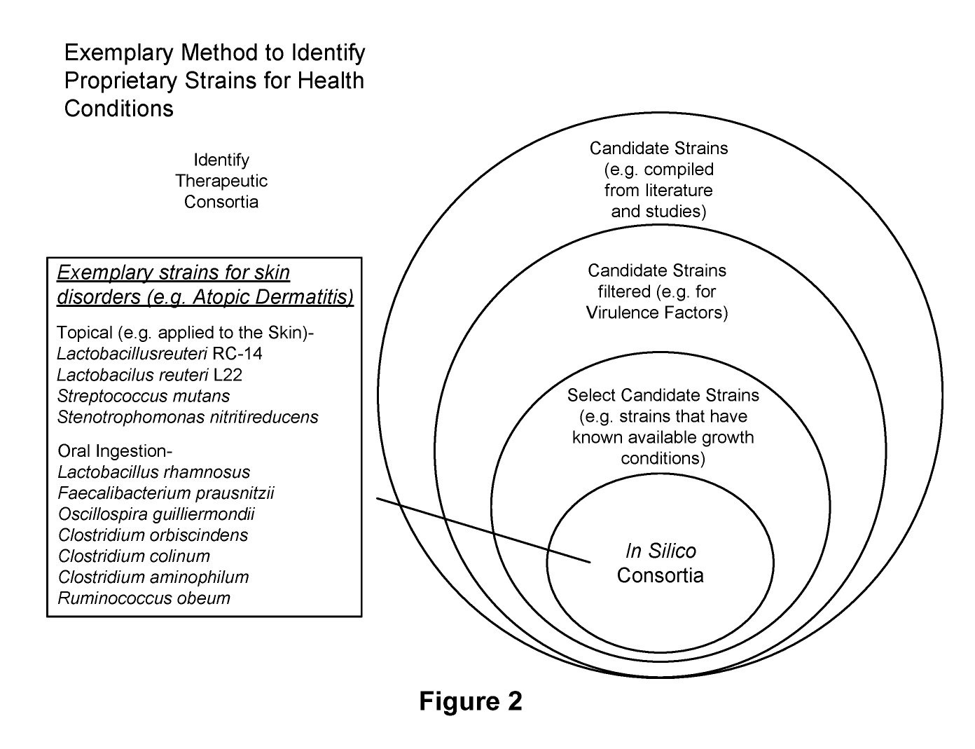

FIG. 2 depicts an illustrative method to identify microorganism strains for use in the treatment of a health condition. A multi-tiered approach can be used to identify one or more microorganism strains for use as a therapeutic. Candidate strains can be found in scientific literature and studies. Candidate strains can be found by analyzing healthy and unhealthy hosts. Candidate strains can be filtered and/or selected for the ability to be administered to a patient (e.g. biosafety level, availability to be manufactured, growth conditions). Finally, an in silico consortia can be determined. Exemplary strains found using this method include, for example, Lactobacillus strains, such as Lactobacillus reuteri RC-14 and Lactobacillus reuteri L22, Stenotrophomonas nitritireducens, Streptococcus mutans, Lactobacillus rhamnosus, Faecalibacterium prausnitzii, Oscillospira guilliermondii, Clostridium orbiscindens, Clostridium colinum, Clostridium aminophilum, and Ruminococcus obeum. In one non-limiting example, microbial strains such as Lactobacillus reuteri RC-14, Lactobacillus reuteri L22, Streptococcus mutans, and/or Stenotrophomonas nitritireducens can be formulated for topical application. In one non-limiting example, microbial strains such as Lactobacillus rhamnosus, Faecalibacterium prausnitzii, Oscillospira guilliermondii, Clostridium orbiscindens, Clostridium colinum, Clostridium aminophilum, and Ruminococcus obeum can be formulated for oral ingestion, for example, as a pill or capsule.

### Methods for Modulating pH

The pH of skin can be from about pH 3.0 to about pH 9.0. The pH can be, for example, about 3, about 3.1, about 3.2, about 3.3, about 3.4, about 3.5, about 3.6, about 3.7, about 3.8, about 3.9, about 4, about 4.1, about 4.2, about 4.3, about 4.4, about 4.5, about 4.6, about 4.7, about 4.8, about 4.9, about 5, about 5.1, about 5.2, about 5.3, about 5.4, about 5.5, about 5.6, about 5.7, about 5.8, about 5.9, about 6, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, about 8.6, about 8.7, about 8.8, about 8.9, or about 9.0. Normal skin pH can be acidic, for example, between about pH 4.0 to about pH 5.5.

Resident (e.g., healthy, normal) skin microbiome can favor acidic conditions. Under acidic skin conditions, for example, from about pH 4.0 to about pH 5.5, the microbiome can remain attached and grow on the skin surface. These conditions can promote resident skin microorganisms to produce various factors, for example, bacteriocins, peptides, metabolites, and hydrogen peroxide, which can inhibit colonization and growth of non-resident and/or pathogenic microorganisms. The resident skin microbiome can also support maintenance of acidic skin surface. A composition of the disclosure can modulate (e.g., reduce, restore, alter, increase) a pH of the subject (e.g., pH of skin) to a pH, for example, an acidic pH (e.g., between about pH 4 and about pH 5.5). A composition of the disclosure can modulate (e.g., reduce, restore, alter, increase) a pH of the subject (e.g., pH of skin) to a pH, for example, at most about pH 6. A composition of the disclosure can modulate (e.g., reduce, restore) a pH of the subject (e.g., pH of skin) to a pH, for example, to about 3, about 3.1, about 3.2, about 3.3, about 3.4, about 3.5, about 3.6, about 3.7, about 3.8, about 3.9, about 4, about 4.1, about 4.2, about 4.3, about 4.4, about 4.5, about 4.6, about 4.7, about 4.8, about 4.9, about 5, about 5.1, about 5.2, about 5.3, about 5.4, about 5.5, about 5.6, about 5.7, about 5.8, about 5.9, about 6, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, about 8.6, about 8.7, about 8.8, about 8.9, or about 9.0 pH units. A composition of the disclosure can modulate (e.g., reduce, restore) a pH of the subject (e.g., pH of skin) to a pH, for example, at least or at most: about 3, about 3.1, about 3.2, about 3.3, about 3.4, about 3.5, about 3.6, about 3.7, about 3.8, about 3.9, about 4, about 4.1, about 4.2, about 4.3, about 4.4, about 4.5, about 4.6, about 4.7, about 4.8, about 4.9, about 5, about 5.1, about 5.2, about 5.3, about 5.4, about 5.5, about 5.6, about 5.7, about 5.8, about 5.9, about 6, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, about 8.6, about 8.7, about 8.8, about 8.9, or about 9.0 pH units.

The pH of the skin can be altered by, for example, soap, detergents, cosmetics, moisturizers, stress, environmental irritants, microbes, and disease conditions. Alkaline skin conditions can include, for example, skin pH above about pH 5, above about pH 5.1, above about pH 5.2, above about pH 5.3, above about pH 5.4, above about pH 5.5, above about pH 5.6, above about pH 5.7, above about pH 5.8, above about pH 5.9, above about pH 6, above about pH 6.1, above about pH 6.2, above about pH 6.3, above about pH 6.4, above about pH 6.5, above about pH 6.6, above about pH 6.7, above about pH 6.8, above about pH 6.9, or above about pH 7. Under alkaline skin conditions (e.g., skin pH above about pH 5.5), resident skin microbiome may not be efficient at inhibiting colonization and growth of non-resident and/or pathogenic microorganisms. This can cause an alteration in the diversity and/or population of microbes in the skin microbiome. The alkaline pH can also result in a weakened skin barrier. Under these conditions, the skin can be susceptible to various infections and disorders, for example atopic dermatitis.

Compositions and methods of the invention can modulate the pH of the skin to allow for the growth and/or restoration of native microbiome. The pH (e.g., skin pH) can be reduced by, for example, about 0.1, about 0.2, about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, or about 4.0 pH units. The pH can be increased by, for example, about 0.1, about 0.2, about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, or about 4.0 pH units.

The pH (e.g., skin pH) can be increased by, for example, about 0.1, about 0.2, about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, or about 4.0 pH units. The pH can be increased by, for example, about 0.1, about 0.2, about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, or about 4.0 pH units.

Compositions of the disclosure can include one or more Lactobacillus species. Non-limiting examples of lactobacillus species include, for example, L. acetotolerans, L. acidifarinae, L. acidipiscis, L. acidophilus, L. agilis, L. algidus, L. alimentarius, L. amylolyticus, L. amylophilus, L. amylotrophicus, L. amylovorus, L. animalis, L. antri, L. apodemi, L. aviarius, L. bifermentans, L. bifidus, L. brevis, L. buchneri, L. bulgaricus, L. camelliae, L. casei, L. catenaformis, L. ceti, L. coleohominis, L. collinoides, L. composti, L. concavus, L. coryniformis, L. crispatus, L. crustorum, L. curvatus, L. delbrueckii subsp. bulgaricus, L. delbrueckii subsp. delbrueckii, L. delbrueckii subsp. lactis, L. dextrinicus, L. diolivorans, L. equi, L. equigenerosi, L. farraginis, L. farciminis, L. fermentum, L. fornicalis, L. fructivorans, L. frumenti, L. fuchuensis, L. gallinarum, L. gasseri, L. gastricus, L. ghanensis, L. graminis, L. hammesii, L. hamsteri, L. harbinensis, L. hayakitensis, L. helveticus, L. hilgardii, L. homohiochii, L. finers, L. ingluviei, L. intestinalis, L. jensenii, L. johnsonii, L. kalixensis, L. kefiranofaciens, L. kefiri, L. kimchii, L. kitasatonis, L. kunkeei, L. leichmannii, L. lindneri, L. malefermentans, L. mali, L. manihotivorans, L. mindensis, L. mucosae, L. murinus, L. nagelii, L. namurensis, L. nantensis, L. oligofermentans, L. oris, L. panis, L. pantheris, L. parabrevis, L. parabuchneri, L. paracasei, L. paracollinoides, L. parafarraginis, L. parakefiri, L. paralimentarius, L. paraplantarum, L. pentosus, L. perolens, L. plantarum, L. pontis, L. protectus, L. psittaci, L. rennini, L. reuteri, L. rhamnosus, L. rimae, L. rogosae, L. rossiae, L. ruminis, L. saerimneri, L. sakei, L. salivarius, L. sanfranciscensis, L. satsumensis, L. secaliphilus, L. sharpeae, L. siliginis, L. spicheri, L. suebicus, L. thailandensis, L. ultunensis, L. vaccinostercus, L. vaginalis, L. versmoldensis, L. vini, L. vitulinus, L. zeae, and L. zymae.

In some embodiments, the lactobacillus strain is L. reuteri RC-14. Lactobacillus reuteri RC-14 probiotic strain can be found, for example in the intestine and vagina. L. reuteri RC-14 can inhibit gram-positive cocci. Biosurfactant capabilities of L. reuteri RC-14 can help reduce pathogen colonization. L. reuteri RC-14 biosurfactant can contain a number of collagen binding proteins that can compete with other microbes, e.g. S. aureus, for binding to host sites. L. reuteri RC-14 can prevent wound sepsis by modulating the immunomodulatory functions.

In some embodiments, the lactobacillus strain is L. reuteri L22. L. reuteri L22 strains can produce hydrogen peroxide and bacteriocins such as reuterin, which can inhibit the growth of other microbes.

Stenotrophomonas species can be abundant in the microbiome of subjects upon treatment with compositions of the invention. Stenotrophomonas species can favor acidic pH conditions.

The compositions can be formulated with live vinegar, for example, vinegar-producing bacteria such as acetic acid bacteria. Non-limiting examples of acetic acid bacteria include species of acetobacter, acidicaldus, acidiphilium, acidisphaera, acidocella, acidomonas, asaia, belnapia, craurococcus, gluconacetobacter, gluconobacter, granulibacter, kozakia, leahibacter, muricoccus, neoasaia, oleomonas, paracraurococcus, rhodopila, roseococcus, rubritepida, saccharibacter, stella, swaminathania, teichococcus, and zavarzinia.

The microbes used in the compositions can be from any suitable source. The microbes can be commensals, naturally-occurring in healthy subjects. The microbes can be symbiotic. The microbes can be genetically altered and/or designed using recombinant strains.

The compositions can be formulated with vinegar. Vinegar is an acidic liquid composed of acetic acid. Owing to the low pH, vinegar can assist in providing growth conditions for microbes that thrive in acidic conditions.

The compositions can be formulated with transurocanic acid. Transurocanic acid can be a breakdown product of filagrrin, which can be a part of the natural system that keeps the pH low on healthy skin. Owing to the low pH, transurocanic acid can assist in providing growth conditions for microbes that thrive in acidic conditions.

The compositions can be formulated with hydrogen peroxide. Hydrogen peroxide can form toxic free radicals and inhibit microbial growth. Hydrogen peroxide can be used at any suitable concentration. The concentration of hydrogen peroxide can be, for example, about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 3%, about 4%, about 5%, about 6%, about 7%, about 8%, about 9%, about 10%, about 11%, about 12%, or about 15%.

The composition can include trans-urocanic acid. Filaggrin can undergo enzymatic degradation to produce trans-urocanic acid. Trans-urocanic acid can contribute to acid-base regulation of the skin. Trans-urocanic acid can maintain a low pH of the skin, thereby promoting a healthy skin microbiome.

The composition can include one or more enzymes, for example, biofilm prevention enzymes and/or adherence prevention enzymes In some embodiments, the compositions include serine protease Esp.

The composition can include a antimicrobial peptide. The composition can include a Phenol-soluble modulin (PSM). Non-limiting examples of PSMs include PSM alpha, PSM alpha 3, PSM beta, PSM gamma, and PSM delta.

The composition can include a bacteriocin. Non-limiting examples of bacteriocins include epidermin, epilancin K7, epilancin 15×, PepS, staphylococcin 1580, reuterin, acidocin, actagardine, agrocin, alveicin, aureocin, aureocin A53, aureocin A70, carnocin, carnocyclin, circularin A, colicin, curvaticin, divercin, duramycin, enterocin, enterolysin, epidermin/gallidermin, erwiniocin, gassericin A, glycinecin, halocin, haloduracin, lactocin S, lactococin, lacticin, leucoccin, macedocin, mersacidin, mesentericin, microbisporicin, microcin S, mutacin, nisin, paenibacillin, planosporicin, pediocin, pentocin, plantaricin, pyocin, reutericin 6, sakacin, salivaricin, subtilin, sulfolobicin, thuricin 17, trifolitoxin, variacin, vibriocin, warnericin, and warnerin.

The compositions can include metabolites for example, to assist in the initial efficacy of the therapeutic before the microbes can produce their own metabolites. Metabolites can include short-chain fatty acids, which can be a subgroup of fatty acids with 6 or less carbons in their aliphatic tails, for example, acetate, propionate, isobutyrate, isovaleric acid, 3-methylbutanoic acid, valeric acid, pentanoic acid, delphinic acid, isopentanoic acid, and butyrate.

The composition can include one or more prebiotics. In one non-limiting example, the prebiotic is an oligosaccharide.

In some embodiments, the prebiotic and probiotic consortia are chosen to create an entirely self-sufficient system that does not require any external input. A combination of probiotics and prebiotics can provide a complete system for producing amino acids, polyphenols, vitamins, and other compounds of nutritive value in a subject. A subject can be treated with a combination of SCFA-producing probiotics and prebiotics comprising dietary fiber and other agents required for the activity of the SCFA-producing probiotics. In this manner, the prebiotic and probiotic form a self-sufficient system, wherein the probiotic converts the prebiotic dietary fiber to SCFAs (butyrate, acetate, propionate), which can trigger downstream signaling for controlling skin disorders in the subject.

### Microbial Compositions

A therapeutic or strain consortia can comprise one or more microorganisms selected from the group consisting of: Akkermansia muciniphila, Anaerostipes caccae, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium coccoides, Clostridium indolis, Clostridium nexile, Clostridium orbiscindens, Clostridium propionicum, Clostridium xylanolyticum, Enterococcus faecium, Eubacterium hallii, Eubacterium rectale, Faecalibacterium prausnitzii, Fibrobacter succinogenes, Lactobacillus acidophilus, Lactobacillus brevis, Lactobacillus bulgaricus, Lactobacillus casei, Lactobacillus caucasicus, Lactobacillus fermentum, Lactobacillus helveticus, Lactobacillus lactis, Lactobacillus plantarum, Lactobacillus reuteri, Lactobacillus rhamnosus, Oscillospira guilliermondii, Roseburia cecicola, Roseburia inulinivorans, Ruminococcus flavefaciens, Ruminococcus gnavus, Ruminococcus obeum, Stenotrophomonas nitritireducens, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus, Coprococcus eutactus, Eubacterium cylindroides, Eubacterium dolichum, Eubacterium ventriosum, Roseburia faeccis, Roseburia hominis, Roseburia intestinalis, Lactobacillus bifidus, Lactobacillus johnsonii, Akkermansia, Bifidobacteria, Clostridia, Eubacteria, Verrucomicrobia, Firmicutes. vinegar-producing bacteria, and any combination thereof.

A therapeutic or strain consortia can comprise one or more microorganisms with at least about: 70%, 75%, 80%, 85%, 87%, 90%, 91%, 92%, 93%, 94%, 95%, 96%, 97%, 98%, 99%, 99.5%, or 100% sequence identity to the rRNA (e.g. 16SrRNA and/or 23S rRNA) of a microorganism selected from the group consisting of: Akkermansia muciniphila, Anaerostipes caccae, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium coccoides, Clostridium indolis, Clostridium nexile, Clostridium orbiscindens, Clostridium propionicum, Clostridium xylanolyticum, Enterococcus faecium, Eubacterium hallii, Eubacterium rectale, Faecalibacterium prausnitzii, Fibrobacter succinogenes, Lactobacillus acidophilus, Lactobacillus brevis, Lactobacillus bulgaricus, Lactobacillus casei, Lactobacillus caucasicus, Lactobacillus fermentum, Lactobacillus helveticus, Lactobacillus lactis, Lactobacillus plantarum, Lactobacillus reuteri, Lactobacillus rhamnosus, Oscillospira guilliermondii, Roseburia cecicola, Roseburia inulinivorans, Ruminococcus flavefaciens, Ruminococcus gnavus, Ruminococcus obeum, Stenotrophomonas nitritireducens, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus, Coprococcus eutactus, Eubacterium cylindroides, Eubacterium dolichum, Eubacterium ventriosum, Roseburia faeccis, Roseburia hominis, Roseburia intestinalis, Lactobacillus bifidus, Lactobacillus johnsonii, Akkermansia, Bifidobacteria, Clostridia, Eubacteria, Verrucomicrobia, Firmicutes. vinegar-producing bacteria, and any combination thereof.

A microbial pharmaceutical composition can comprise a therapeutically-effective amount of a population of isolated and purified microbes, wherein the population of isolated and purified microbes comprises one or more microbes with a rRNA (e.g., 16SrRNA and/or 23S rRNA) sequence comprising at least about: 70%, 75%, 80%, 85%, 87%, 90%, 91%, 92%, 93%, 94%, 95%, 96%, 97%, 98%, 99%, 99.5%, or 100% sequence identity to a rRNA sequence from a microbe selected from the group consisting of: Akkermansia muciniphila, Anaerostipes caccae, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium coccoides, Clostridium indolis, Clostridium nexile, Clostridium orbiscindens, Clostridium propionicum, Clostridium xylanolyticum, Enterococcus faecium, Eubacterium hallii, Eubacterium rectale, Faecalibacterium prausnitzii, Fibrobacter succinogenes, Lactobacillus acidophilus, Lactobacillus brevis, Lactobacillus bulgaricus, Lactobacillus casei, Lactobacillus caucasicus, Lactobacillus fermentum, Lactobacillus helveticus, Lactobacillus lactis, Lactobacillus plantarum, Lactobacillus reuteri, Lactobacillus rhamnosus, Oscillospira guilliermondii, Roseburia cecicola, Roseburia inulinivorans, Ruminococcus flavefaciens, Ruminococcus gnavus, Ruminococcus obeum, Stenotrophomonas nitritireducens, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus, Coprococcus eutactus, Eubacterium cylindroides, Eubacterium dolichum, Eubacterium ventriosum, Roseburia faeccis, Roseburia hominis, Roseburia intestinalis, Lactobacillus bifidus, Lactobacillus johnsonii, Akkermansia, Bifidobacteria, Clostridia, Eubacteria, Verrucomicrobia, Firmicutes. vinegar-producing bacteria, and any combination thereof.

In some embodiments, provided are pharmaceutical compositions to treat a disorder (e.g., a skin disorder) comprising a therapeutically-effective amount of a population of isolated and purified microbes, wherein the population of isolated and purified microbes comprises one or more microbes with a rRNA (e.g., 16SrRNA and/or 23S rRNA) sequence comprising at least about: 70%, 75%, 80%, 85%, 87%, 90%, 91%, 92%, 93%, 94%, 95%, 96%, 97%, 98%, 99%, 99.5%, or 100% sequence identity to a rRNA sequence from a microbe selected from the group consisting of: Akkermansia muciniphila, Anaerostipes caccae, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium coccoides, Clostridium indolis, Clostridium nexile, Clostridium orbiscindens, Clostridium propionicum, Clostridium xylanolyticum, Enterococcus faecium, Eubacterium hallii, Eubacterium rectale, Faecalibacterium prausnitzii, Fibrobacter succinogenes, Lactobacillus acidophilus, Lactobacillus brevis, Lactobacillus bulgaricus, Lactobacillus casei, Lactobacillus caucasicus, Lactobacillus fermentum, Lactobacillus helveticus, Lactobacillus lactis, Lactobacillus plantarum, Lactobacillus reuteri, Lactobacillus rhamnosus, Oscillospira guilliermondii, Roseburia cecicola, Roseburia inulinivorans, Ruminococcus flavefaciens, Ruminococcus gnavus, Ruminococcus obeum, Stenotrophomonas nitritireducens, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus, Coprococcus eutactus, Eubacterium cylindroides, Eubacterium dolichum, Eubacterium ventriosum, Roseburia faeccis, Roseburia hominis, Roseburia intestinalis, Lactobacillus bifidus, Lactobacillus johnsonii, Akkermansia, Bifidobacteria, Clostridia, Eubacteria, Verrucomicrobia, Firmicutes. vinegar-producing bacteria, and any combination thereof.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from an Lactobacillus species.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from an Akkermansia.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from a Bifidobacterium.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from a Clostridium.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from a Eubacterium,

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from a Verrucomicrobium,

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from a Firmicute.

In some embodiments, provided are pharmaceutical microbial compositions comprising a therapeutically-effective amount of a population of isolated and purified microbes, wherein the population of isolated and purified microbes comprises one or more microbes with a rRNA (e.g., 16SrRNA and/or 23S rRNA) sequence comprising at least about: 70%, 75%, 80%, 85%, 87%, 90%, 91%, 92%, 93%, 94%, 95%, 96%, 97%, 98%, 99%, 99.5%, or 100% sequence identity to a rRNA sequence from a microbe selected from the group consisting of: Lactobacillus reuteri (e.g., Lactobacillus reuteri RC-14, Lactobacillus reuteri L22), Streptococcus mutans, Stenotrophomonas nitritireducens, and any combination thereof.

In some embodiments, provided are pharmaceutical microbial compositions comprising a therapeutically-effective amount of a population of isolated and purified microbes, wherein the population of isolated and purified microbes comprises one or more microbes with a rRNA (e.g., 16SrRNA and/or 23S rRNA) sequence comprising at least about: 70%, 75%, 80%, 85%, 87%, 90%, 91%, 92%, 93%, 94%, 95%, 96%, 97%, 98%, 99%, 99.5%, or 100% sequence identity to a rRNA sequence from a microbe selected from the group consisting of: Lactobacillus rhamnosus, Faecalibacterium prausnitzii, Oscillospira guilliermondii, Clostridium orbiscindens, Clostridium colinum, Clostridium aminophilum, Ruminococcus obeum, and any combination thereof.

In some embodiments, provided are pharmaceutical microbial compositions comprising a therapeutically-effective amount of a population of isolated and purified microbes, wherein the population of isolated and purified microbes comprises one or more microbes with a rRNA (e.g., 16SrRNA and/or 23S rRNA) sequence comprising at least about: 70%, 75%, 80%, 85%, 87%, 90%, 91%, 92%, 93%, 94%, 95%, 96%, 97%, 98%, 99%, 99.5%, or 100% sequence identity to a rRNA sequence from a microbe selected from the group consisting of: Akkermansia muciniphila, Bifidobacterium adolescentis, Bifidobacterium infantis, Bifidobacterium longum, Clostridium beijerinckii, Clostridium butyricum, Clostridium indolis, Eubacterium hallii, and any combination thereof.

In some embodiments, provided are pharmaceutical microbial compositions comprising a therapeutically-effective amount of a population of isolated and purified microbes, wherein the population of isolated and purified microbes comprises one or more microbes with a rRNA (e.g., 16SrRNA and/or 23S rRNA) sequence comprising at least about: 70%, 75%, 80%, 85%, 87%, 90%, 91%, 92%, 93%, 94%, 95%, 96%, 97%, 98%, 99%, 99.5%, or 100% sequence identity to a rRNA sequence from a microbe selected from the group consisting of: Akkermansia muciniphila, Bifidobacterium adolescentis, Bifidobacterium infantis, Bifidobacterium longum, Clostridium beijerinckii, Clostridium butyricum, Clostridium indolis, Eubacterium hallii, Faecalibacterium prausnitzii, and any combination thereof.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Akkermansia muciniphila.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Anaerostipes caccae.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Bifidobacterium adolescentis.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Bifidobacterium bifidum.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Bifidobacterium infantis

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Bifidobacterium longum.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Butyrivibrio fibrisolvens.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Clostridium acetobutylicum.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Clostridium aminophilum.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Clostridium beijerinckii.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Clostridium butyricum.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Clostridium colinum.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Clostridium coccoides.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Clostridium indolis.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Clostridium nexile.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Clostridium orbiscindens.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Clostridium propionicum.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Clostridium xylanolyticum.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Enterococcus faecium.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Eubacterium hallii.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Eubacterium rectale.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Faecalibacterium prausnitzii.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Fibrobacter succinogenes.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Lactobacillus acidophilus.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Lactobacillus brevis.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Lactobacillus bulgaricus.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Lactobacillus casei.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Lactobacillus caucasicus.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Lactobacillus fermentum.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Lactobacillus helveticus.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Lactobacillus lactis.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Lactobacillus plantarum

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Lactobacillus reuteri.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Lactobacillus rhamnosus.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Oscillospira guilliermondii.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Roseburia cecicola.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Roseburia inulinivorans.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Ruminococcus flavefaciens.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Ruminococcus gnavus.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Ruminococcus obeum.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Stenotrophomonas nitritireducens.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Streptococcus cremoris.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Streptococcus faecium.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Streptococcus infantis.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Streptococcus mutans.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Streptococcus thermophdus.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Anaerofustis stercorihominis.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Anaerostipes hadrus.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Anaerotruncus colihominis.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Clostridium sporogenes.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Clostridium tetani.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Coprococcus.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Coprococcus eutactus.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Eubacterium cylindroides.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Eubacterium dolichum.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Eubacterium ventriosum.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Roseburia faeccis

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Roseburia hominis.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Roseburia intestinalis.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from a vinegar-producing microbe.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Lactobacillus bifidus.

In one embodiment, a pharmaceutical composition comprises a therapeutically-effective amount of an isolated and/or purified microbe with a rRNA (e.g., 16S rRNA and/or 23S rRNA) sequence comprising at least about: 85%, 87%, 90%, 92%, 95%, 96%, 97%, 98%, 99%, 99.5%, or 100% sequence identity to a rRNA sequence from Lactobacillus johnsonii

A therapeutic composition can comprise at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 45, or at least 50, or at least 75, or at least 100 types of bacteria. A therapeutic composition can comprise at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 21, at most 22, at most 23, at most 24, at most 25, at most 26, at most 27, at most 28, at most 29, at most 30, at most 31, at most 32, at most 33, at most 34, at most 35, at most 36, at most 37, at most 38, at most 39, at most 40, at most 45, or at most 50, or at most 75, or at most 100 types of bacteria.

In some embodiments, combining one or more microbes in a therapeutic composition or consortia increases or maintains the stability of the microbes in the composition compared with the stability of the microbes alone. A therapeutic consortium of microbes can provide a synergistic stability compared with the individual strains.

In some embodiments, combining one or more microbes in a therapeutic composition or consortia can provide a synergistic effect when administered to the individual. For example, administration of a first microbe may be beneficial to a subject and administration of a second microbe may be beneficial to a subject but when the two microbes are administered together to a subject, the benefit is greater than the either benefit alone.

Different types of microbes in a therapeutic composition can be present in the same amount or in different amounts. For example, the ratio of two bacteria in a therapeutic composition can be about 1:1, 1:2, 1:5, 1:10, 1:25, 1:50, 1:100, 1:1000, 1:10,000, or 1:100,000.

### Microbial Growth and Production

Microorganisms of the invention can be produced in any suitable medium for growth, some non-limiting examples include: RCM, GYT veg, BHI, PYGveg, nutrient media, minimal media, selective media, differential media, and transport media. The growth medium can comprise a trace mineral. The growth medium can comprise a salt. The growth medium can comprise a vitamin. The growth medium can comprise a buffer. The pH of a growth medium can be, for example, about 7. The pH of a growth medium can be, for example, about 3, about, 4, about, 5, about 6, about 7, or about 8. The growth medium can improve the maximum density a microbial strain can grow to. The growth medium can allow for higher strain concentrations. The growth medium can buffer acid production by a microbial strain, which can minimize the inhibitory effect of, for example, very low pH.

Microorganisms of the invention can be grown in aerobic growth conditions. Microorganisms of the invention can be grown in anaerobic growth conditions.

Table 1 shows trace minerals that can be added to a growth media:

TABLE 1
Trace minerals

| Trace minerals | mg/L |
|---|---|
| component | medium |
| CoCl₂ | 0.65 |
| CuCl₂*2H₂O | 0.03 |
| H₃BO₃ | 3.52 |
| FeSO₄*7H₂O | 1.50 |
| MnCl₂*4H₂O | 0.26 |
| Na₂EDTA | 25.01 |
| Na₂MoO₄*2H₂O | 0.80 |
| Na₂SeO₃ | 0.39 |
| NiCl₂ | 0.65 |
| ZnSO₄*7H₂O | 0.29 |

Table 2 shows vitamins that can be added to a growth media. The concentrations shown in Table 2 can be final concentrations in the growth media.

TABLE 2
Vitamin solution.

| Vitamin Solution | mg/L |
|---|---|
| component | medium |
| D-biotin | 0.2 |
| Ca-pantothenate | 2.5 |
| myoinositol | 20 |
| p-aminobenzoic acid | 0.5 |
| pyridoxine | 5 |
| hydrochloride | |
| riboflavine | 0.5 |
| thiamine dichloride | 10 |
| vitamin B12 | 0.2 |
| nicotinic acid | 5 |

Table 3 shows an illustrative growth medium:

TABLE 3
Illustrative growth medium recipe

| GYTveg broth (per liter): | | |
|---|---|---|
| Component | Amount | |
| Glucose | 10 | g |
| HiVeg Hydrolysate | 5 | g |
| Yeast Extract | 10 | g |
| Na-thioglycolate | 0.5 | g |
| Resazurin | 80 μl (of 14 g/l stock) | |
| Vitamin solution | 10 | ml |
| Agar (for solid medium) | 18 | g |

Table 4 shows an illustrative growth medium.

TABLE 4
Illustrative growth medium recipe

| GYTveg + CaCO₃ | | |
|---|---|---|
| (per liter): | | |
| Component | Amount | |
| Glucose | 10 | g |
| HiVeg Hydrolysate | 5 | g |
| Yeast Extract | 10 | g |
| Na-thioglycolate | 0.5 | g |
| Resazurin | 80 μl (of 14 g/l stock) | |
| Vitamin solution | 10 | ml |
| CaCO₃ | 20 | g |
| Agar (for solid medium) | 18 | g |

Table 5 shows an illustrative growth medium.

TABLE 5
Illustrative growth medium recipe

| PYGveg | | |
|---|---|---|
| Component | Amount per liter | |
| Glucose | 5 | g |
| K₂HPO₄ | 2 | g |
| Tween 80 | 1 | ml |
| Cystein-HCl | 0.5 | g |
| Yeast extract | 10 | g |
| HiVeg Extract | 5 | g |
| HiVeg Peptone #1 | 5 | g |
| HiVeg Peptone #3 | 5 | g |
| Vitamin Mix 100x | 10 | ml |
| Salt solution | 40 | ml |

Table 6 shows illustrative salts that can be added to a growth medium. The concentrations shown in Table 6 can be final concentrations in the growth medium.

TABLE 6
Salt solution

| Salt solution | |
|---|---|
| Component | grams per liter |
| CaCl₂ 2H₂O | 0.02 |
| MgSO₄ 7H₂O | 0.02 |
| K₂HPO₄ | 0.04 |
| KH₂PO₄ | 0.04 |
| NaHCO₃ | 0.4 |
| NaCl | 0.08 |

In some embodiments, the growth medium comprises PYGveg (e.g., Table 5), vitamins (e.g., Table 2), salt (e.g., Table 6), and a buffer.

An assay can be used to determine and/or measure growth of a microbe, for example, in a growth medium. The assay can be used to quantitate live cells. The assay can be used to determine a ratio of live vs dead cells (e.g., percentage live/dead cells).

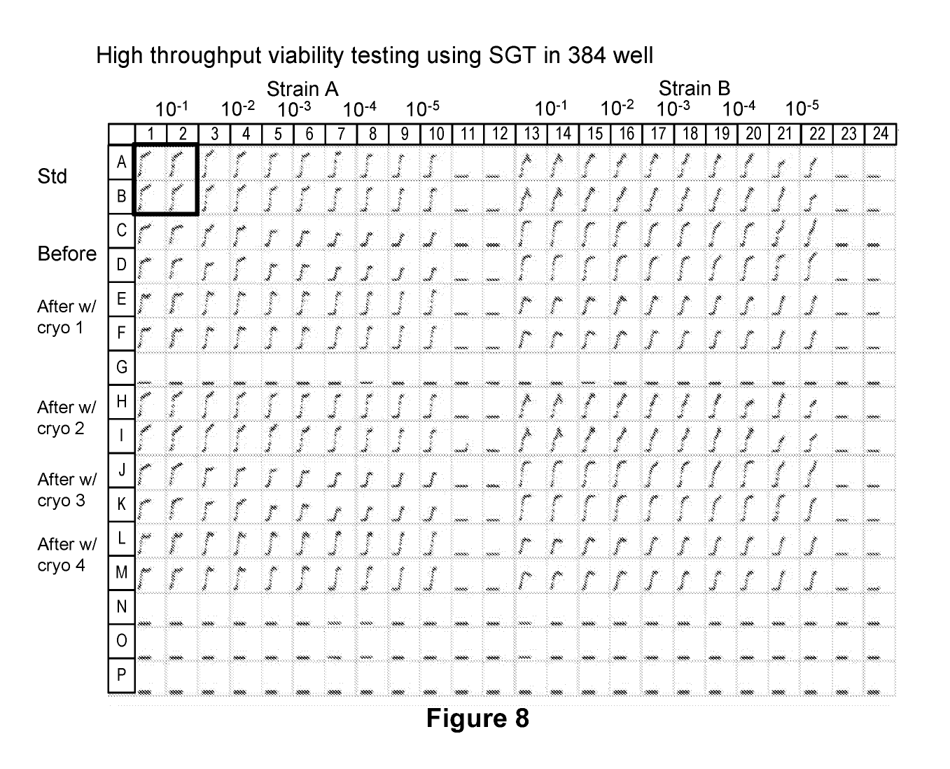

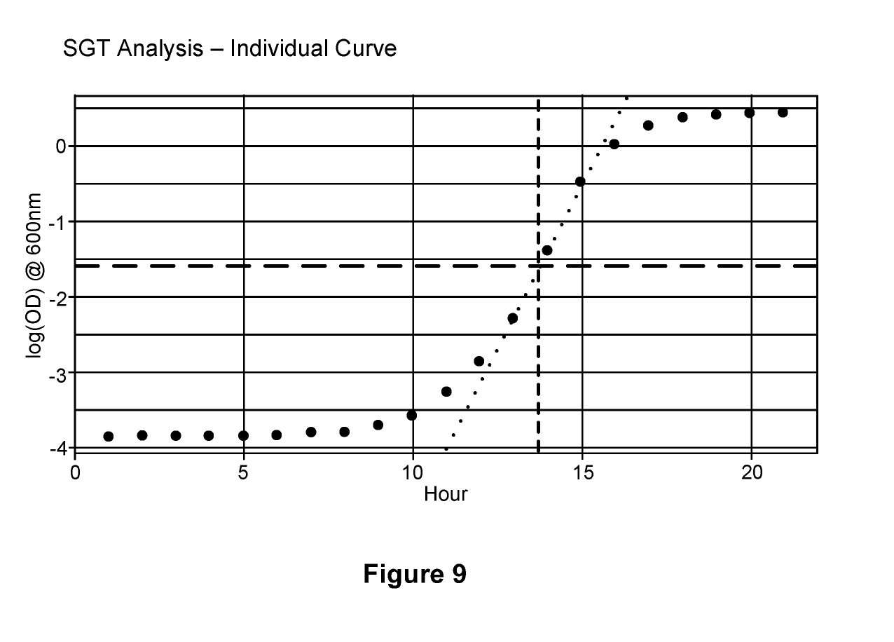

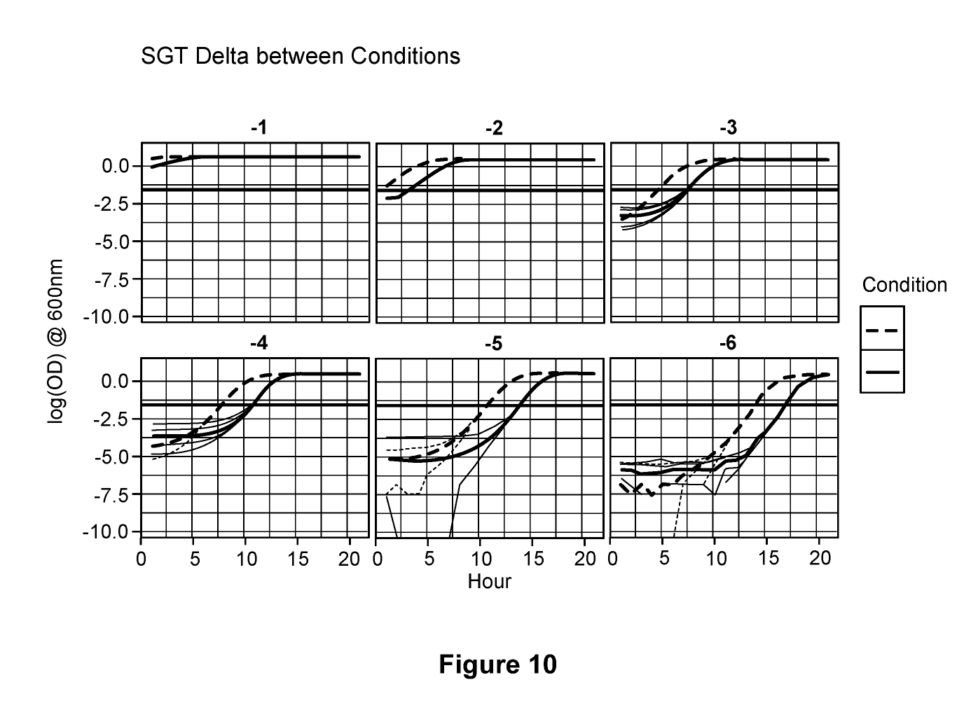

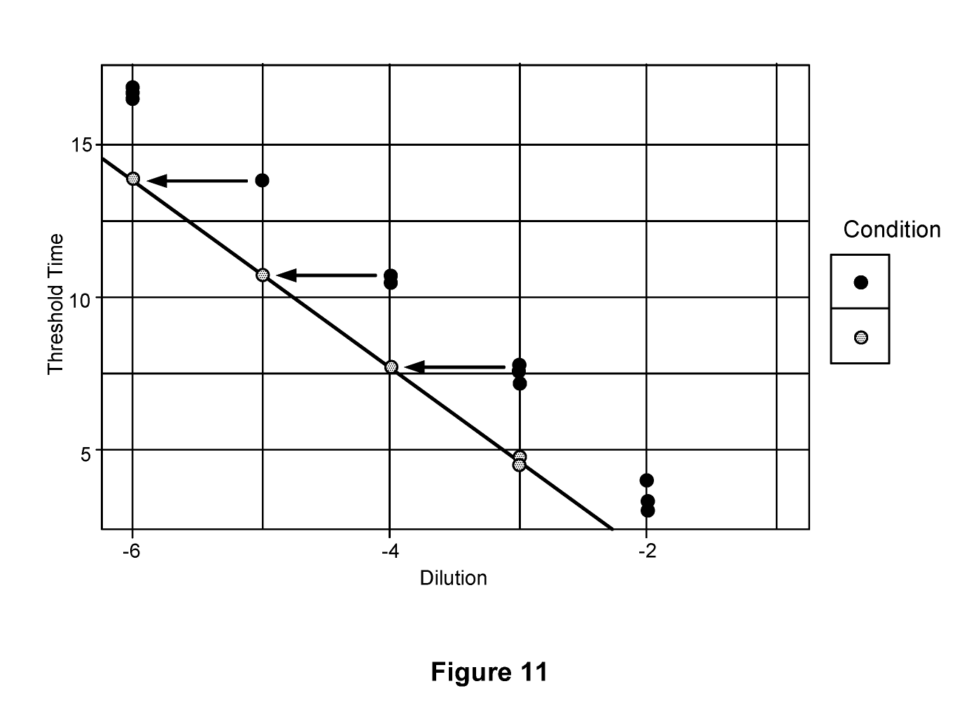

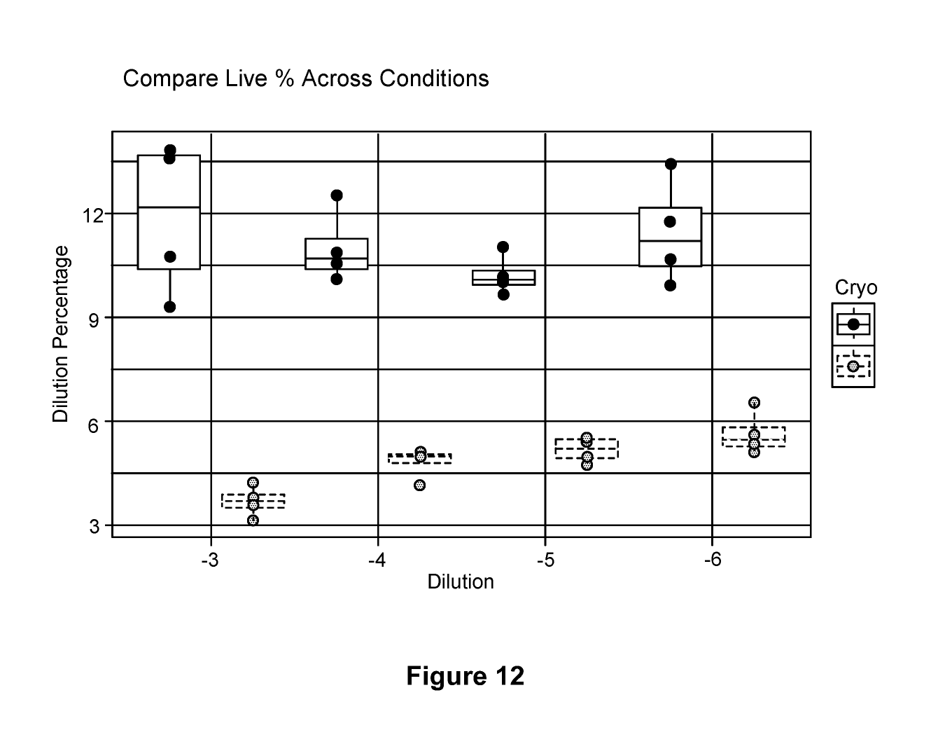

A non-limiting example of an assay that can be used to measure growth of a microbe is a start growth time (SGT) assay. A Start Growth Time (SGT) assay can be based on the re-growth time required by a growing cell culture to reach a threshold (e.g., a threshold Optical Density OD₆₀₀). The growth time required to reach the threshold can be proportional to the number of viable cells in the initial inoculum. FIGS. 8-14 illustrate examples of growth assays.

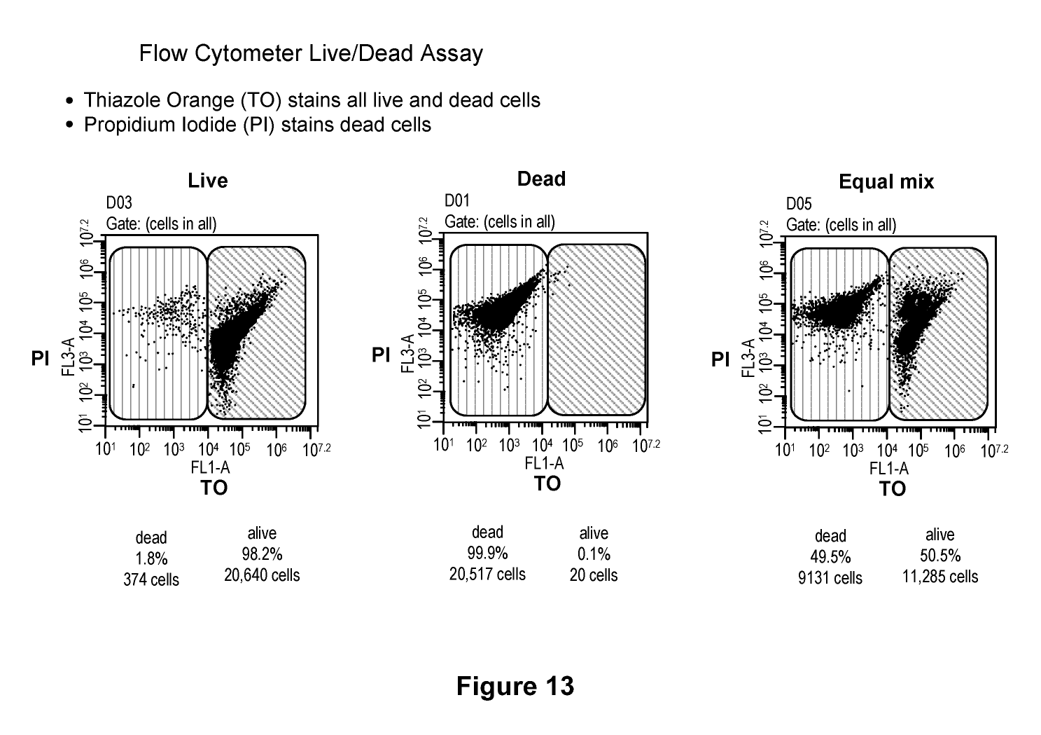

In another non-limiting example of an assay for measuring growth of cells and quantitate live cells, cells can be treated with one or more agents that differentiates a live cell from a dead cell. For example, one or more dyes can be used to color dead and/or live cells as illustrated in FIG. 13. A thaizole orange dye can be used to stain live and dead cells, for example, green. A propidium iodide dye can be used to stain dead cells, for example, red. The stained cells can be analyzed using a flow cytometer to quantitate live cells as compared to dead cells.

### Pharmaceutical Compositions

Provided herein are compositions that may be administered as therapeutics and/or cosmetics. One or more microorganisms described herein can be used to create a pharmaceutical formulation comprising an effective amount of the composition for treating a subject. The microorganisms can be in any suitable formulation. Some non-limiting examples can include topical, capsule, pill, enema, liquid, injection, and the like. In some embodiments, the one or more strains disclosed herein may be included in a food or beverage product, cosmetic, or nutritional supplement.

A pharmaceutical composition of the invention can be a combination of any microorganisms described herein with other components, such as carriers, stabilizers, diluents, dispersing agents, suspending agents, thickening agents, and/or excipients. The pharmaceutical composition can facilitate administration of the microorganisms to a subject. Pharmaceutical compositions can be administered in therapeutically-effective amounts as pharmaceutical compositions by various forms and routes including, for example, oral, topical, rectal, transdermal, mucosal, and vaginal administration. A combination of administration routes can be utilized. The pharmaceutical composition can be administered as therapeutics and/or cosmetics.

The composition can be administered by a suitable method to any suitable body part or body surface of the subject, for example, that shows a correlation with a skin disorder.

In some embodiments, the composition is administered to the skin of the subject. Non-limiting examples of skin or skin surface include cheeks, scalp, trunk, extremities, flexural areas, elbows, knees, neck, wrists, hands, eyelids, stomach, arms, chin, and feet.

Skin can include, for example, skin surface, epidermis, stratum corneum, stratum lucidem, stratum granulosum, stratum spinosum, stratum basale, dermis, connective tissue, hair follicles, hair shaft, sebaceous gland, sweat pore, and sweat glands.

In some embodiments, the composition is administered to a part of the gastrointestinal tract of a subject. Non-limiting examples of parts of gastrointestinal tract include oral cavity, mouth, esophagus, stomach, duodenum, small intestine regions including duodenum, jejunum, ileum, and large intestine regions including cecum, colon, rectum, and anal canal. In some embodiments, the composition is formulated for delivery to the ileum and/or colon regions of the gastrointestinal tract. In some embodiments, the composition is administered to multiple body parts or surfaces, for example, skin and gut.

The composition can include one or more active ingredients. Active ingredients can be selected from the group consisting of: metabolites, bacteriocins, enzymes, anti-microbial peptides, antibiotics, prebiotics, probiotics, glycans (as decoys that would limit specific bacterial/viral binding to the intestinal wall), bacteriophages, and microorganisms.

In some embodiments, the formulation comprises a prebiotic. In some embodiments, the prebiotic is inulin. In some embodiments, the prebiotic is a fiber. The prebiotic, for example, inulin can serve as an energy source for the microbial formulation.

The compositions can be administered topically. The compositions can be formulated as a topically administrable composition, such as solutions, suspensions, lotions, gels, pastes, medicated sticks, balms, creams, ointments, liquid, wrap, adhesive, or patch. The compositions can contain solubilizers, stabilizers, tonicity enhancing agents, buffers and preservatives.

The compositions can be administered orally, for example, through a capsule, pill, powder, tablet, gel, or liquid, designed to release the composition in the gastrointestinal tract.

In some embodiments, administration of a formulation occurs by injection, for example, for a formulation comprising, for example, butyrate, propionate, acetate, and short-chain fatty acids. In some embodiments, administration of a formulation occurs by a suppository and/or by enema. In some embodiments, a combination of administration routes is utilized.

Microbial compositions can be formulated as a dietary supplement. Microbial compositions can be incorporated with vitamin supplements. Microbial compositions can be formulated in a chewable form such as a probiotic gummy. Microbial compositions can be incorporated into a form of food and/or drink. Non-limiting examples of food and drinks where the microbial compositions can be incorporated include, for example, bars, shakes, juices, infant formula, beverages, frozen food products, fermented food products, and cultured dairy products such as yogurt, yogurt drink, cheese, acidophilus drinks, and kefir.

A formulation of the disclosure can be administered as part of a fecal transplant process. A formulation can be administered to a subject by a tube, for example, nasogastric tube, nasojejunal tube, nasoduodenal tube, oral gastric tube, oral jejunal tube, or oral duodenal tube. A formulation can be administered to a subject by colonoscopy, endoscopy, sigmoidoscopy, and/or enema.

In some embodiments, the microbial composition is formulated such that the one or more microbes can replicate once they are delivered to the target habitat (e.g. skin, gut). In some embodiments, the microbial composition is formulated such that the one or more microbes are viable in the target habitat (e.g., skin, gut). In one non-limiting example, the microbial composition is formulated in a pill, such that the pill has a shelf life of at least about: 1, 2, 3, 4, 5, 6, 7, 8, 9, 10, 11, or 12 months. In another non-limiting example, the storage of the microbial composition is formulated so that the microbes can reproduce in the target habitat, e.g, skin and/or gut. In some embodiments, other components may be added to aid in the shelf life of the microbial composition. In some embodiments, one or more microbes may be formulated in a manner that it is able to survive in a non-natural environment. For example, a microbe that is native to the gut may not survive in an oxygen-rich environment. To overcome this limitation, the microbe may be formulated in a pill that can reduce or eliminate the exposure to oxygen. Other strategies to enhance the shelf-life of microbes may include other microbes (e.g. if the bacterial consortia comprises a composition whereby one or more strains is helpful for the survival of one or more strains).

In some embodiments, a microbial composition is lyophilized (e.g., freeze-dried) and formulated as a powder, tablet, enteric-coated capsule (e.g. for delivery to the gut such as ileum and/or colon region), or pill that can be administered to a subject by any suitable route. The lyophilized formulation can be mixed with a saline or other solution prior to administration.

In some embodiments, a microbial composition is formulated for oral administration, for example, as an enteric-coated capsule or pill, for delivery of the contents of the formulation to the ileum and/or colon regions of a subject.

In some embodiments, the microbial composition is formulated for oral administration. In some embodiments, the microbial composition is formulated as an enteric-coated pill or capsule for oral administration. In some embodiments, the microbial composition is formulated for delivery of the microbes to the ileum region of a subject. In some embodiments, the microbial composition is formulated for delivery of the microbes to the colon region (e.g. upper colon) of a subject. In some embodiments, the microbial composition is formulated for delivery of the microbes to the ileum and colon (e.g., upper colon) regions of a subject.

An enteric-coating can protect the contents of a formulation, for example, oral formulation such as pill or capsule, from the acidity of the stomach. An enteric-coating can provide delivery to the ileum and/or upper colon regions. A microbial composition can be formulated such that the contents of the composition may not be released in a body part other than the gut region, for example, ileum and/or colon region of the subject. Non-limiting examples of enteric coatings include pH sensitive polymers (e.g., eudragit FS30D), methyl acrylate-methacrylic acid copolymers, cellulose acetate succinate, hydroxy propyl methyl cellulose phthalate, hydroxy propyl methyl cellulose acetate succinate (e.g., hypromellose acetate succinate), polyvinyl acetate phthalate (PVAP), methyl methacrylate-methacrylic acid copolymers, shellac, cellulose acetate trimellitate, sodium alginate, zein, other polymers, fatty acids, waxes, shellac, plastics, and plant fibers. In some embodiments, the enteric coating is formed by a pH sensitive polymer. In some embodiments, the enteric coating is formed by eudragit FS30D.

The enteric coating can be designed to dissolve at any suitable pH. In some embodiments, the enteric coating is designed to dissolve at a pH greater than from about pH 6.5 to about pH 7.0. In some embodiments, the enteric coating is designed to dissolve at a pH greater than about pH 6.5. In some embodiments, the enteric coating is designed to dissolve at a pH greater than about pH 7.0. The enteric coating can be designed to dissolve at a pH greater than about: 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, or 7.5 pH units. The enteric coating can be designed to dissolve in the gut, for example, ileum and/or colon region. The enteric coating can be designed to not dissolve in the stomach.

The formulation can be stored in cold storage, for example, at a temperature of about −80° C., about −20° C., about −4° C., or about 4° C. Compositions provided herein can be stored at any suitable temperature. The storage temperature can be, for example, about 0° C., about 1° C., about 2° C., about 3° C., about 4° C., about 5° C., about 6° C., about 7° C., about 8° C., about 9° C., about 10° C., about 12° C., about 14° C., about 16° C., about 20° C., about 22° C., or about 25° C. In some embodiments, the storage temperature is between about 2° C. to about 8° C. Storage of microbial compositions at low temperatures, for example from about 2° C. to about 8° C., can keep the microbes alive and increase the efficiency of the composition. The cooling conditions can also provide soothing relief to patients. Storage at freezing temperature, below 0° C., with a cryoprotectant can further extend stability.

A composition of the disclosure can be at any suitable pH. The pH of the composition can range from about 3 to about 12. The pH of the composition can be, for example, from about 3 to about 4, from about 4 to about 5, from about 5 to about 6, from about 6 to about 7, from about 7 to about 8, from about 8 to about 9, from about 9 to about 10, from about 10 to about 11, or from about 11 to about 12 pH units. The pH of the composition can be, for example, about 3, about 4, about 5, about 6, about 7, about 8, about 9, about 10, about 11, or about 12 pH units. The pH of the composition can be, for example, at least 3, at least 4, at least 5, at least 6, at least 7, at least 8, at least 9, at least 10, at least 11 or at least 12 pH units. The pH of the composition can be, for example, at most 3, at most 4, at most 5, at most 6, at most 7, at most 8, at most 9, at most 10, at most 11, or at most 12 pH units. The pH of the composition can be, for example, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, or about 7.0 pH units. If the pH is outside the range desired by the formulator, the pH can be adjusted by using sufficient pharmaceutically-acceptable acids and bases. In some embodiments, the pH of the composition is from about 4 to about 6 pH units. In some embodiments, the pH of the composition is about 5.5 pH units.

Microbial compositions can be formulated as a dietary supplement. Microbial compositions can be incorporated with vitamin supplements. Microbial compositions can be formulated in a chewable form such as a probiotic gummy. Microbial compositions can be incorporated into a form of food and/or drink. Non-limiting examples of food and drinks where the microbial compositions can be incorporated include, for example, bars, shakes, juices, infant formula, beverages, frozen food products, fermented food products, and cultured dairy products such as yogurt, yogurt drink, cheese, acidophilus drinks, and kefir.

A composition of the disclosure can be administered as part of a fecal transplant process. A composition can be administered to a subject by a tube, for example, nasogastric tube, nasojejunal tube, nasoduodenal tube, oral gastric tube, oral jejunal tube, or oral duodenal tube. A composition can be administered to a subject by colonoscopy, endoscopy, sigmoidoscopy, and/or enema.

In some embodiments, a microbial composition is lyophilized (freeze-dried) and formulated as a powder, tablet, enteric-coated capsule, or pill that can be administered to a subject by any suitable route, for example, oral, enema, suppository, injection. The lyophilized composition can be mixed with a saline or other solution prior to administration.

In some embodiments, the administration of a composition of the disclosure can be preceded by, for example, colon cleansing methods such as colon irrigation/hydrotherapy, enema, administration of laxatives, dietary supplements, dietary fiber, enzymes, and magnesium.

In some embodiments, the microbes are formulated as a population of spores. Spore-containing compositions can be administered by any suitable route described herein. Orally administered spore-containing compositions can survive the low pH environment of the stomach. The amount of spores employed can be, for example, from about 1% w/w to about 99% w/w of the entire composition.

Compositions provided herein can include the addition of one or more agents to the therapeutics or cosmetics in order to enhance stability and/or survival of the microbial composition. Non-limiting example of stabilizing agents include genetic elements, glycerin, ascorbic acid, skim milk, lactose, tween, alginate, xanthan gum, carrageenan gum, mannitol, palm oil, and poly-L-lysine (POPL).

In some embodiments, a composition comprises recombinant microbes or microbes that have been geneticallly modified. In some embodiments, the composition comprises microbes that can be regulated, for example, a microbe comprising an operon to control microbial growth.

A composition can be customized for a subject. A custom composition can comprise, for example, a prebiotic, a probiotic, an antibiotic, or a combination of active agents described herein. Data specific to the subject comprising for example age, gender, and weight can be combined with an analysis result to provide a therapeutic agent customized to the subject. For example, a subject's microbiome found to be low in a specific microbe relative to a sub-population of healthy subjects matched for age and gender can be provided with a therapeutic and/or cosmetic composition comprising the specific microbe to match that of the sub-population of healthy subjects having the same age and gender as the subject.

In some embodiments, a composition is administered before, during, and/or after treatment with an antimicrobial agent such as an antibiotic. For example, the composition can be administered at least 1 hour, 2 hours, 5 hours, 12 hours, 1 day, 3 days, 1 week, 2 weeks, 1 month, 6 months, or 1 year before and/or after treatment with an antibiotic. The composition can be administered at most 1 hour, 2 hours, 5 hours, 12 hours, 1 day, 3 days, 1 week, 2 weeks, 1 month, 6 months, or 1 year before and/or after treatment with an antibiotic.

In some embodiments, the formulation is administered after treatment with an antibiotic. For example, the formulation can be administered after the entire antibiotic regimen or course is complete.

In some embodiments, a formulation is administered before, during, and/or after food intake by a subject. In some embodiments, the formulation is administered with food intake by the subject. In some embodiments, the formulation is administered with (e.g., simultaneously) with food intake.

In some embodiments, the formulation is administered before food intake by a subject. In some embodiments, the formulation is more effective or potent at treating a microbial condition when administered before food intake. For example, the formulation can be administered about 1 minute, about 2 minutes, about 3 minutes, about 5 minutes, about 10 minutes, about 15 minutes, about 30 minutes, about 45 minutes, about 1 hour, about 2 hours, about 3 hours, about 4 hours, about 5 hours, about 6 hours, about 7 hours, about 8 hours, about 9 hours, about 10 hours, about 12 hours, or about 1 day before food intake by a subject. For example, the formulation can be administered at least about 1 minute, about 2 minutes, about 3 minutes, about 5 minutes, about 10 minutes, about 15 minutes, about 30 minutes, about 45 minutes, about 1 hour, about 2 hours, about 3 hours, about 4 hours, about 5 hours, about 6 hours, about 7 hours, about 8 hours, about 9 hours, about 10 hours, about 12 hours, or about 1 day before food intake by a subject. For example, the formulation can be administered at most about 1 minute, about 2 minutes, about 3 minutes, about 5 minutes, about 10 minutes, about 15 minutes, about 30 minutes, about 45 minutes, about 1 hour, about 2 hours, about 3 hours, about 4 hours, about 5 hours, about 6 hours, about 7 hours, about 8 hours, about 9 hours, about 10 hours, about 12 hours, or about 1 day before food intake by a subject.

In some embodiments, the formulation is administered after food intake by the subject. In some embodiments, the formulation is more effective or potent at treating a microbial condition when administered after food intake. For example, the formulation can be administered at least about 1 minute, 2 minutes, 3 minutes, 5 minutes, 10 minutes, 15 minutes, 30 minutes, 45 minutes, 1 hour, 2 hours, 3 hours, 5 hours, 10 hours, 12 hours, or 1 day after food intake by a subject. For example, the formulation can be administered at most about 1 minute, 2 minutes, 3 minutes, 5 minutes, 10 minutes, 15 minutes, 30 minutes, 45 minutes, 1 hour, 2 hours, 3 hours, 5 hours, 10 hours, 12 hours, or 1 day after food intake by a subject.

Formulations provded herein can include those suitable for oral including buccal and sub-lingual, intranasal, topical, transdermal, transdermal patch, pulmonary, vaginal, rectal, suppository, mucosal, systemic, or parenteral including intramuscular, intraarterial, intrathecal, intradermal, intraperitoneal, subcutaneous, and intravenous administration or in a form suitable for administration by aerosolization, inhalation or insufflation.

A therapeutic or cosmetic composition can include carriers and excipients (including but not limited to buffers, carbohydrates, lipids, mannitol, proteins, polypeptides or amino acids such as glycine, antioxidants, bacteriostats, chelating agents, suspending agents, thickening agents and/or preservatives), metals (e.g., iron, calcium), salts, vitamins, minerals, water, oils including those of petroleum, animal, vegetable or synthetic origin, such as peanut oil, soybean oil, mineral oil, sesame oil and the like, saline solutions, aqueous dextrose and glycerol solutions, flavoring agents, coloring agents, detackifiers and other acceptable additives, adjuvants, or binders, other pharmaceutically acceptable auxiliary substances as required to approximate physiological conditions, such as pH buffering agents, tonicity adjusting agents, emulsifying agents, wetting agents and the like. Examples of excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Non-limiting examples of pharmaceutically-acceptable excipients suitable for use in the disclosure include granulating agents, binding agents, lubricating agents, disintegrating agents, sweetening agents, glidants, anti-adherents, anti-static agents, surfactants, anti-oxidants, gums, coating agents, coloring agents, flavouring agents, dispersion enhancer, disintegrant, coating agents, plasticizers, preservatives, suspending agents, emulsifying agents, plant cellulosic material and spheronization agents, and any combination thereof.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins1999), each of which is incorporated by reference in its entirety.

A composition can be substantially free of preservatives. In some applications, the compositon may contain at least one preservative.

A composition can be encapsulated within a suitable vehicle, for example, a liposome, a microspheres, or a microparticle. Microspheres formed of polymers or proteins can be tailored for passage through the gastrointestinal tract directly into the blood stream. Alternatively, the compound can be incorporated and the microspheres, or composite of microspheres, and implanted for slow release over a period of time ranging from days to months.

A composition can be formulated as a sterile solution or suspension. The therapeutic or cosmetic compositions can be sterilized by conventional techniques or may be sterile filtered. The resulting aqueous solutions may be packaged for use as is, or lyophilized. The lyophilized preparation of the microbial composition can be packaged in a suitable form for oral administration, for example, capsule or pill.

The compositions can be administered topically and can be formulated into a variety of topically administrable compositions, such as solutions, suspensions, lotions, gels, pastes, medicated sticks, balms, creams, and ointments. Such pharmaceutical compositions can contain solubilizers, stabilizers, tonicity enhancing agents, buffers and preservatives.

The compositions can also be formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as a mixture of fatty acid glycerides, optionally in combination with cocoa butter, can be used.

Microbial compositions can be formulated using one or more physiologically-acceptable carriers comprising excipients and auxiliaries, which facilitate processing of the microorganisms into preparations that can be used pharmaceutically. Compositions can be modified depending upon the route of administration chosen. Compositions described herein can be manufactured in a conventional manner, for example, by means of conventional mixing, dissolving, granulating, dragee-making, levigating, encapsulating, entrapping, emulsifying or compression processes.

Pharmaceutical compositions containing microbes described herein can be administered for prophylactic and/or therapeutic treatments. In therapeutic applications, the compositions can be administered to a subject already suffering from a disease or condition, in an amount sufficient to cure or at least partially arrest the symptoms of the disease or condition, or to cure, heal, improve, or ameliorate the condition. Microbial compositions can also be administered to lessen a likelihood of developing, contracting, or worsening a condition. Amounts effective for this use can vary based on the severity and course of the disease or condition, previous therapy, the subject's health status, weight, and response to the drugs, and the judgment of the treating physician.

Multiple therapeutic agents can be administered in any order or simultaneously. If simultaneously, the multiple therapeutic agents can be provided in a single, unified form, or in multiple forms, for example, as multiple separate pills. The composition can be packed together or separately, in a single package or in a plurality of packages. One or all of the therapeutic agents can be given in multiple doses. If not simultaneous, the timing between the multiple doses may vary to as much as about a month.

Compositions described herein can be administered before, during, or after the occurrence of a disease or condition, and the timing of administering the composition can vary. For example, the microbial composition can be used as a prophylactic and can be administered continuously to subjects with a propensity to conditions or diseases in order to lessen a likelihood of the occurrence of the disease or condition. The microbial compositions can be administered to a subject during or as soon as possible after the onset of the symptoms. The administration of the microbial compositions can be initiated within the first 48 hours of the onset of the symptoms, within the first 24 hours of the onset of the symptoms, within the first 6 hours of the onset of the symptoms, or within 3 hours of the onset of the symptoms. The initial administration can be via any route practical, such as by any route described herein using any composition described herein. A microbial composition can be administered as soon as is practicable after the onset of a disease or condition is detected or suspected, and for a length of time necessary for the treatment of the disease, such as, for example, from about 1 month to about 3 months. The length of treatment can vary for each subject.

Compositions of the invention can be administered in combination with another therapy, for example, immunotherapy, chemotherapy, radiotherapy, anti-inflammatory agents, anti-viral agents, anti-microbial agents, and anti-fungal agents.

Compositions of the invention can be packaged as a kit. In some embodiments, a kit includes written instructions on the administration/use of the composition. The written material can be, for example, a label. The written material can suggest conditions methods of administration. The instructions provide the subject and the supervising physician with the best guidance for achieving the optimal clinical outcome from the administration of the therapy. The written material can be a label. In some embodiments, the label can be approved by a regulatory agency, for example the U.S. Food and Drug Administration (FDA), the European Medicines Agency (EMA), or other regulatory agencies.

### Dosage

The appropriate quantity of a therapeutic or cosmetic composition to be administered, the number of treatments, and unit dose can vary according to a subject and/or the disease state of the subject.

Pharmaceutical compositions described herein can be in unit dosage forms suitable for single administration of precise dosages. In unit dosage form, the formulation can be divided into unit doses containing appropriate quantities of one or more microbial compositions. The unit dosage can be in the form of a package containing discrete quantities of the formulation. Non-limiting examples are liquids in vials or ampoules. Aqueous suspension compositions can be packaged in single-dose non-reclosable containers. The composition can be in a multi-dose format. Multiple-dose reclosable containers can be used, for example, in combination with a preservative. Formulations for parenteral injection can be presented in unit dosage form, for example, in ampoules, or in multi-dose containers with a preservative.

The dosage can be in the form of a solid, semi-solid, or liquid composition. Non-limiting examples of dosage forms suitable for use in the invention include feed, food, pellet, lozenge, liquid, elixir, aerosol, inhalant, spray, powder, tablet, pill, capsule, gel, geltab, nanosuspension, nanoparticle, microgel, suppository troches, aqueous or oily suspensions, ointment, patch, lotion, dentifrice, emulsion, creams, drops, dispersible powders or granules, emulsion in hard or soft gel capsules, syrups, phytoceuticals, nutraceuticals, dietary supplement, and any combination thereof.

A microbe can be present in any suitable concentration in a pharmaceutical composition. The concentration of a microbe can be for example, from about 10¹ to about 10¹⁸ colony forming units (CFU). The concentration of a microbe can be, for example, about 10¹, about 10², about 10³, about 10⁴, about 10⁵, about 10⁶, about 10⁷, about 10⁸, about 10⁹, about 10¹⁰, about 10¹¹, about 10¹², about 10¹³ about 10¹⁴, about 10¹⁵, about 10¹⁶, about 10¹⁷, or about 10¹⁸ CFU. The concentration of a microbe can be, for example, at least about 10¹, at least about 10², at least about 10³, at least about 10⁴, at least about 10⁵, at least about 10⁶, at least about 10⁷, at least about 10⁸, at least about 10⁹, at least about 10¹⁰, at least about 10¹¹, at least about 10¹², at least about 10¹³, at least about 10¹⁴, at least about 10¹⁵, at least about 10¹⁶, at least about 10¹⁷, or at least about 10¹⁸ CFU. The concentration of a microbe can be, for example, at most about 10¹, at most about 10², at most about 10³, at most about 10⁴, at most about 10⁵, at most about 10⁶, at most about 10⁷, at most about 10⁸, at most about 10⁹, at most about 10¹⁰, at most about 10¹¹, at most about 10¹², at most about 10¹³, at most about 10¹⁴, at most about 10¹⁵, at most about 10¹⁶, at most about 10¹⁷, or at most about 10¹⁸ CFU. In some embodiments, the concentration of a microbe is from about 10⁸ CFU to about 10⁹ CFU. In some embodiments, the concentration of a microbe is about 10⁸ CFU. In some embodiments, the concentration of a microbe is about 10⁹ CFU. In some embodiments, the concentration of a microbe is about 10¹⁰ CFU. In some embodiments, the concentration of a microbe is at least about 10⁸ CFU. In some embodiments, the concentration of a microbe is at least about 10⁹ CFU.

The concentration of a microbe in a formulation can be equivalent to, for example, about: 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 25, 30, 35, 40, 45, 50, 55, 60, 70, 80, 90, or 100 OD units. The concentration of a microbe in a formulation can be equivalent to, for example, at least about: 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 25, 30, 35, 40, 45, 50, 55, 60, 70, 80, 90, or 100 OD units. The concentration of a microbe in a formulation can be equivalent to, for example, at most about: 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 25, 30, 35, 40, 45, 50, 55, 60, 70, 80, 90, or 100 OD units.

Pharmaceutical compositions of the invention can be formulated with any suitable therapeutically-effective concentration of an active ingredient. For example, the therapeutically-effective concentration of a prebiotic can be at least about 1 mg/ml, about 2 mg/ml, about 3 mg/ml, about 4 mg/ml, about 5 mg/ml, about 10 mg/ml, about 15 mg/ml, about 20 mg/ml, about 25 mg/ml, about 30 mg/ml, about 35 mg/ml, about 40 mg/ml, about 45 mg/ml, about 50 mg/ml, about 55 mg/ml, about 60 mg/ml, about 65 mg/ml, about 70 mg/ml, about 75 mg/ml, about 80 mg/ml, about 85 mg/ml, about 90 mg/ml, about 95 mg/ml, about 100 mg/ml, about 110 mg/ml, about 125 mg/ml, about 130 mg/ml, about 140 mg/ml, or about 150 mg/ml. For example, the therapeutically-effective concentration of a prebiotic can be at most about 1 mg/ml, about 2 mg/ml, about 3 mg/ml, about 4 mg/ml, about 5 mg/ml, about 10 mg/ml, about 15 mg/ml, about 20 mg/ml, about 25 mg/ml, about 30 mg/ml, about 35 mg/ml, about 40 mg/ml, about 45 mg/ml, about 50 mg/ml, about 55 mg/ml, about 60 mg/ml, about 65 mg/ml, about 70 mg/ml, about 75 mg/ml, about 80 mg/ml, about 85 mg/ml, about 90 mg/ml, about 95 mg/ml, about 100 mg/ml, about 110 mg/ml, about 125 mg/ml, about 130 mg/ml, about 140 mg/ml, or about 150 mg/ml. For example, the therapeutically-effective concentration of a prebiotic can be about 1 mg/ml, about 2 mg/ml, about 3 mg/ml, about 4 mg/ml, about 5 mg/ml, about 10 mg/ml, about 15 mg/ml, about 20 mg/ml, about 25 mg/ml, about 30 mg/ml, about 35 mg/ml, about 40 mg/ml, about 45 mg/ml, about 50 mg/ml, about 55 mg/ml, about 60 mg/ml, about 65 mg/ml, about 70 mg/ml, about 75 mg/ml, about 80 mg/ml, about 85 mg/ml, about 90 mg/ml, about 95 mg/ml, about 100 mg/ml, about 110 mg/ml, about 125 mg/ml, about 130 mg/ml, about 140 mg/ml, or about 150 mg/ml. In some embodiments, the concentration of a prebiotic in a pharmaceutical composition is about 70 mg/ml. In some embodiments, the prebiotic is inulin.

Pharmaceutical compositions of the invention can be administered, for example, 1, 2, 3, 4, 5, or more times daily. Pharmaceutical compositions of the invention can be administered, for example, daily, every other day, three times a week, twice a week, once a week, or at other appropriate intervals for treatment of the condition.

In practicing the methods of treatment or use provided herein, therapeutically-effective amounts of the compounds described herein are administered in pharmaceutical compositions to a subject having a disease or condition to be treated. A therapeutically-effective amount can vary widely depending on the severity of the disease, the age and relative health of the subject, the potency of the compounds used, and other factors.

Subjects can be, for example, mammal, humans, elderly adults, adults, adolescents, pre-adolescents, children, toddlers, infants, newborn, or neonates. A subject can be a patient. In some embodiments, a subject is a human. In some embodiments, a subject is a child (i.e. a young human being below the age of puberty). In some embodiments, a subject is an infant. A subject can be an individual enrolled in a clinical study. A subject can be a laboratory animal, for example, a mammal, or a rodent.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

Additionally or alternatively to any of the above-disclosed embodiments, the disclosure comprises the following enumerated embodiments.

Embodiment 1 is a method of treating a skin disorder in a subject in need thereof, the method comprising administering to the subject a pharmaceutical composition comprising a therapeutically-effective amount of a population of isolated and purified microorganisms. Optionally the purified microorganisms are purified microbes. Optionally, the composition further comprises a pharmaceutically acceptable carrier. Optionally, the skin disorder is atopic dermatitis, inflammation, alergy, or any combination thereof. Optionally, the subject is a human. Optionally, the subject is a child. Optionally, the method further comprises determining a microbiome profile of the subject. Optionally, the method further comprises diagnosing a condition of the subject based on the microbiome profile. Optionally, wherein said treating results in the subject having increased butyrate production as compared to a pre-treatment level. Optionally, the method further comprises determining a composition of a microbiome of the subject.

Embodiment 2 is a pharmaceutical composition comprising a therapeutically effective amount of a population of isolated and purified microorganisms. Optionally, the pharmaceutical composition has a pH of about 5.5.

Embodiment 3 is the method or composition of either Embodiment 1 or 2 wherein at least one of said microorganisms is of at least one of the following: a recombinant microorganism, capable of modulating a pH of the subject, capable of modulating the pH of the skin of the subject, capable of producing vinegar, capable of producing vinegar is an acetic acid bacterium, capable of producing hydrogen peroxide, capable of altering the microbiome in said subject, capable of modulating the gut microbiome, capable of restoring the gut microbiome, capable of modulating butyrate production capable of reducing pH of said subject, capable of reducing pH to between about pH 4.0 and about pH 5.0, capable of stimulating a toll-like receptor, or any combination thereof.

Embodiment 4 is the method or composition of any of Embodiments 1-3, wherein at least one of said microorganisms comprises a microorganism selected from the group consisting of: Akkermansia muciniphila, Anaerostipes caccae, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium coccoides, Clostridium indolis, Clostridium nexile, Clostridium orbiscindens, Clostridium propionicum, Clostridium xylanolyticum, Enterococcus faecium, Eubacterium hallii, Eubacterium rectale, Faecalibacterium prausnitzii, Fibrobacter succinogenes, Lactobacillus acidophilus, Lactobacillus brevis, Lactobacillus bulgaricus, Lactobacillus casei, Lactobacillus caucasicus, Lactobacillus fermentum, Lactobacillus helveticus, Lactobacillus lactis, Lactobacillus plantarum, Lactobacillus reuteri, Lactobacillus rhamnosus, Oscillospira guilliermondii, Roseburia cecicola, Roseburia inulinivorans, Ruminococcus flavefaciens, Ruminococcus gnavus, Ruminococcus obeum, Stenotrophomonas nitritireducens, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus, Coprococcus eutactus, Eubacterium cylindroides, Eubacterium dolichum, Eubacterium ventriosum, Roseburia faeccis, Roseburia hominis, Roseburia intestinalis, Lacatobacillus bifidus, Lactobacillus johnsonii, and any combination thereof. Additionally or alternatively, said Lactobacillus reuteri is selected from the group consisting of: Lactobacillus reuteri RC-14, Lactobacillus reuteri L22, and any combination thereof.

Embodiment 5 is the method or composition of any of Embodiments 1-4, wherein the pharmaceutical composition is formulated for at least one of the following: oral administration, for topical administration, as a pill, as a tablet, as a capsule, as a patch, as a wrap, as a lotion, as a cream, or any combination thereof.

Embodiment 6 is the method or composition of any of Embodiments 1-5, wherein the pharmaceutical composition further comprises at last one of the following: hydrogen peroxide, comprises vinegar, trans-urocanic acid, a metabolite, an anti-microbial peptide, phenol-soluble modulin (PSM), PSM gamma, PSM delta, bacteriocin, an enzyme, a serine protease, a probiotic, a prebiotic, an oligosaccharide, inulin, inulin in an amount of at least 50 mg/ml, or any combination thereof. Optionally, the metabolite is selected from the group consisting of acetate, propionate, isobutyrate, isovaleric acid, 3-methylbutanoic acid, valeric acid, pentanoic acid, delphinic acid, isopentanoic acid, and butyrate. Optionally, the bacteriocin is selected from the group consisting of: epidermin, epilancin K7, epilancin 15×, pep5, staphylococcin 1580, and any combination thereof. Optionally, the prebiotic selected from the group consisting of: complex carbohydrates, complex sugars, resistant dextrins, resistant starch, amino acids, peptides, nutritional compounds, biotin, polydextrose, oligosaccharides, polysaccharide, fructooligosaccharide (FOS), fructans, soluble fiber, insoluble fiber, fiber, starch, galactooligosaccharides (GOS), inulin, lignin, psyllium, chitin, chitosan, gums, high amylose cornstarch (HAS), cellulose, β-glucans, hemi-celluloses, lactulose, mannooligosaccharides, mannan oligosaccharides (MOS), oligofructose-enriched inulin, oligofructose, oligodextrose, tagatose, trans-galactooligosaccharide, pectin, resistant starch, and xylooligosaccharides (XOS), and any combination thereof.

Embodiment 7 is the method or composition of any of Embodiments 1-6, wherein the population of isolated and purified microbes within the pharmaceutical composition comprises a microbe with a ribosomal RNA (rRNA) sequence comprising at least about 85% sequence identity to a rRNA sequence selected from the group consisting of: Akkermansia muciniphila, Bifidobacterium adolescentis, Bifidobacterium infantis, Bifidobacterium longum, Clostridium beijerinckii, Clostridium butyricum, Clostridium indolis, Eubacterium hallii, Faecalibacterium prausnitzii, and any combination thereof.

Embodiment 8 is the method or composition of any of Embodiments 1-6, wherein the population of isolated and purified microbes within the pharmaceutical composition comprises a microbe with a ribosomal RNA (rRNA) sequence comprising at least about 85% sequence identity to a rRNA sequence selected from the group consisting of: Lactobacillus reuteri, Streptococcus mutans, Stenotrophomonas nitritireducens, and any combination thereof.

Embodiment 9 is the method or composition of any of Embodiments 1-6, wherein the population of isolated and purified microbes within the pharmaceutical composition comprises a microbe with a ribosomal RNA (rRNA) sequence comprising at least about 85% sequence identity to a rRNA sequence selected from the group consisting of: Lactobacillus rhamnosus, Faecalibacterium prausnitzii, Oscillospira guilliermondii, Clostridium orbiscindens, Clostridium colinum, Clostridium aminophilum, and Ruminococcus obeum, and any combination thereof.

Embodiment 10 is the method or composition of any of Embodiments 1-6, wherein the population of isolated and purified microbes comprises a microbe with a ribosomal RNA (rRNA) sequence comprising at least about 85% sequence identity to a rRNA sequence from at least one of the following: Lactobacillus rhamnosus, Oscillospira guilliermondii, Clostridium orbiscindens, Clostridium colinum, Clostridium aminophilum, Ruminococcus obeum, Akkermansia muciniphila, Bifidobacterium adolescentis, Bifidobacterium infantis, Bifidobacterium longum, Clostridium beijerinckii, Clostridium butyricum, Clostridium indolis, Eubacterium hallii, Faecalibacterium prausnitzii, Lactobacillus reuteri, Streptococcus mutans, Stenotrophomonas nitritireducens, or any combination thereof.

Embodiment 11 is the method or composition of any of Embodiments 1-10, wherein the pharmaceutical composition is formulated for oral delivery. Optionally, the pharmaceutical composition is formulated as a pill, tablet, or capsule. Optionally, the oral composition has an enteric-coating. Optionally, the enteric coating dissolves at a pH greater than at least about pH 6.5. Optionally, the pharmaceutical composition is delivered to a small intestine of the subject. Optionally, the pharmaceutical composition is delivered to an ileum of the small intestine of the subject. Optionally, the pharmaceutical composition is delivered to a large intestine of the subject. Optionally, the pharmaceutically composition is administered before food intake by the subject. Additionally or alternatively, the pharmaceutically composition is administered with food intake by the subject.

Embodiment 12 is the method or composition of any of Embodiments 1-11, wherein the therapeutically-effective amount of each purified and isolated microbe in the pharmaceutical composition is at least about 10⁵ colony forming units (CFU). Additionally or alternatively, the pharmaceutical composition is administered after completion of an antibiotic regimen by the subject. Additionally or alternatively, the pharmaceutical composition does not substantially release the population of isolated and purified microbes prior to a small intestine of the subject.

### EXAMPLES

### Example 1

### Microbial Compositions in Modulating Skin pH

Background:

The pH of skin can be affected by external irritants and environmental stresses. Changes in pH can affect the native microbiome of the skin and disrupt the stratum corneum barrier leading to inflammation, infection, and skin disorders.

Objective:

The purpose of the study is to assess the effect of microbial compositions of the invention in restoring normal skin pH upon exposure to external irritants that lead to alkaline conditions.

Methods:

Twenty subjects without skin disease, enter an open, controlled and randomized study.
1) Experimental group: Ten subjects have skin washed with alkaline soap (pH 9.5) followed by topical application of a microbial composition having a pH of about 5.5, comprising Lactobacillus reuteri, Streptococcus mutans, Stenotrophomonas nitritireducens, hydrogen peroxide, vinegar, acetic-acid bacteria, trans-urocanic acid, PSM gamma, PSM delta, epidermin, epilancin K7, epilancin 15×, pep5, and staphylococcin 1580, and serine protease Esp, once a day for 3 weeks. Test area is cheek.
2) Control group: Ten subjects have skin washed with alkaline soap (pH 9.5) followed by topical application of a control composition, for example, PBS or an alternative buffer, once a day for 3 weeks. Test area is cheek.

The pH and microbiome profile of the treated area on the cheek are assessed at regular intervals before washing with soap and at regular intervals post-topical application, for example, on Day 0 [baseline], Day 7, Day 14, and Day 21. Statistical analysis, for example, using Student's t test, is used to assess the significance of the results in experimental and control group.

For determining the microbiome profile, samples are collected in the form of skin swabs from the treated cheek area. Briefly, a sterile swab is first dipped into a tube containing sterile 1×PBS to wet; the swab is then swiped across the area of interest 10-20 times; next the swab is gently dipped into 300 uL of lysis buffer, containing 20 mM Tris, pH8.0, 20 mM EDTA, 1% SDS, 0.5% Tween, 1% Triton X-100, and 400 ug/mL proteinase K, in a sterile 1.5 mL tube; and the swab is left in the microcentrifuge tube until nucleic acid extraction. The subsequent extraction of DNA and removal of exonuclease is conducted followed by adapter ligation at various higher concentrations. Size-selection using Ampure and Blue Pippen approaches are performed to enrich for the expected length amplicon species. After extraction, the nucleic acid samples are selected for the proper size. PCR amplification reactions are conducted to prepare the libraries for sequencing. Forward and reverse primers are selected based on empirical data that indicate which sets had minimal self-complimentarity. Finally, samples are sequenced using long read length sequence technology.

The alkaline soap can influence the skin surface by increasing the pH. This can result in a change in the cutaneous microbial population, which can favor an acidic pH. The main efficacy outcome for the study is maintenance of acidic pH conditions and native skin microbiome following administration of the microbial composition.

Subjects in the experimental group have a normal acidic skin pH and microbiome profile. Subjects in the control group have a significantly high skin pH, altered skin microbiome profile, and start developing skin disorders.

### Example 2

### Treatment a Skin Disorder (e.g., Pediatric Atopic Dermatitis) with a Microbial Composition

A four-year old subject is brought to a pediatrician. The subject complains of itching and has red patches on the hands, face, scalp, feet, ankles, wrists, neck, upper chest, eyelids, and inside the bend of the elbows and knees. The affected skin of the subject is thickened, cracked, dry, and scaly. The subject also has bumps that leak fluid and crust over when scratched. The subject is diagnosed with atopic dermatitis by the pediatrician.

The pediatrician prescribes a microbial-based lotion comprising Lactobacillus reuteri, Streptococcus mutans, Stenotrophomonas nitritireducens, hydrogen peroxide, vinegar, acetic-acid bacteria, trans-urocanic acid, PSM gamma, PSM delta, epidermin, epilancin K7, epilancin 15×, pep5, and staphylococcin 1580, and serine protease Esp. The composition is to be administered topically on the affected skin area twice daily for fourteen consecutive days.

Application of the microbial-based lotion provides a significant reduction and/or disappearance of atopic dermatitis-related symptoms of the subject.

### Example 3

### Microbial Compositions in Modulating Gut Microbiome to Treat a Skin Disorder (e.g. Atopic Dermatitis)

Background:

The gut microbiome can be involved with training the immune system. Dysbiosis of the gut microbiome can therefore lead to many downstream inflammation-based reactions that can manifest in several ways, including on the skin as with atopic dermatitis. Correcting the gut dysbiosis can therefore alleviate downstream inflammation of the skin. Microbial compositions that modulate gut microbiome can reduce, for example, inflammation and allergic reactions, which can lead to a skin disorder (e.g. atopic dermatitis).

Objective:

The purpose of the study is to assess the effect of microbial compositions of the invention in restoring normal gut microbiome function and/or treat skin disorders.

Methods:

Twenty subjects with skin disease, enter a double-blind, placebo controlled and randomized study.
1) Experimental group: Ten subjects are given oral compositions containing the active composition comprising: Lactobacillus rhamnosus, Faecalibacterium prausnitzii, Oscillospira guilliermondii, Clostridium orbiscindens, Clostridium colinum, Clostridium aminophilum, and Ruminococcus obeum. The composition is taken once a day for 3 weeks. Parameters observed are number, rate, and severity of atopic dermatitis breakouts.
2) Control group: Ten subjects are given a placebo pill. The placebo is taken once a day for 3 weeks. Parameters observed are number, rate, and severity of atopic dermatitis breakouts.

Following treatment, subjects in the experimental group have a restored gut microbiome and significantly lower number, rate, and severity of atopic dermatitis breakouts compared with the control group.

### Example 4

### Computer Systems

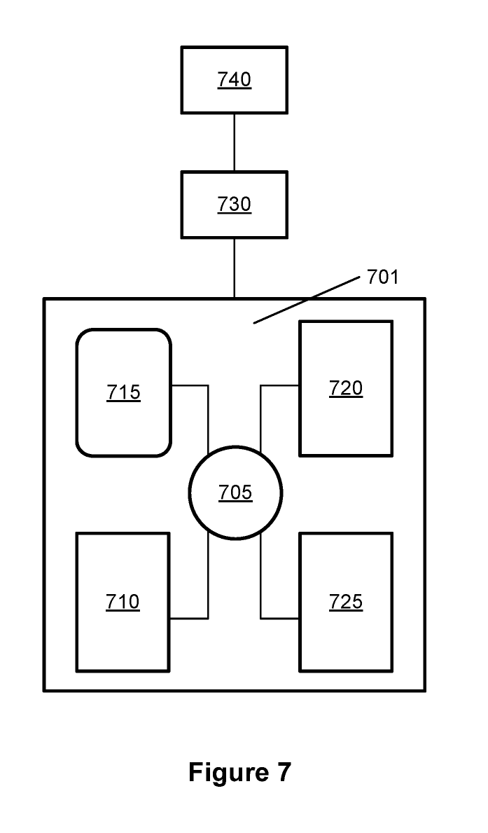

The invention also provides a computer system that is configured to implement the methods of the disclosure. The system can include a computer server (“server”) that is programmed to implement the methods described herein. FIG. 7 depicts a adapted to enable a user to detect, analyze, and process data (e.g. sequencing data; strain classification, functional pathways, epigenetic changes, patient information, external data, databases, microbiome strains; therapeutic consortia, etc.). The system 700 includes a central computer server 701 that is programmed to implement exemplary methods described herein. The server 701 includes a central processing unit (CPU, also “processor”) 705 which can be a single core processor, a multi core processor, or plurality of processors for parallel processing, or cloud processors. The server 701 also includes memory 710 (e.g. random access memory, read-only memory, flash memory); electronic storage unit 715 (e.g. hard disk); communications interface 720 (e.g. network adaptor) for communicating with one or more other systems; and peripheral devices 725 which may include cache, other memory, data storage, and/or electronic display adaptors. The memory 710, storage unit 715, interface 720, and peripheral devices 725 are in communication with the processor 705 through a communications bus (solid lines), such as a motherboard. The storage unit 715 can be a data storage unit for storing data. The server 701 is operatively coupled to a computer network (“network”) 730 with the aid of the communications interface 720. The network 730 can be the Internet, an intranet and/or an extranet, an intranet and/or extranet that is in communication with the Internet, a telecommunication or data network. The network 730 in some cases, with the aid of the server 701, can implement a peer-to-peer network, which may enable devices coupled to the server 701 to behave as a client or a server. Peripheral devices can include, e.g. sequencers 725 or remote computer systems 740.

The storage unit 715 can store files, (e.g. any aspect of data associated with the invention). In some instances cloud storage is used. Cloud storage can be a model of data storage where the digital data is stored in logical pools, wherein the physical storage can span multiple servers and, in some instances, one or more locations. In some embodiments, the physical environment is owned and managed by a hosting company. Cloud storage services may be accessed, e.g., through a co-located cloud compute service, a web service application programming interface (API) or by applications that utilize the API, such as cloud desktop storage, a cloud storage gateway or Web-based content management systems.

The server can communicate with one or more remote computer systems through the network 730. The one or more remote computer systems may be, for example, personal computers, laptops, tablets, telephones, Smart phones, or personal digital assistants.

In some situations the system 700 includes a single server 701. In other situations, the system includes multiple servers in communication with one another through an intranet, extranet and/or the Internet.

The server 701 can be adapted to store information. Such information can be stored on the storage unit 715 or the server 701 and such data can be transmitted through a network.

Methods as described herein can be implemented by way of machine (e.g., computer processor) computer readable medium (or software) stored on an electronic storage location of the server 701, such as, for example, on the memory 710, or electronic storage unit 715. During use, the code can be executed by the processor 705. In some cases, the code can be retrieved from the storage unit 715 and stored on the memory 710 for ready access by the processor 705. In some situations, the electronic storage unit 715 can be precluded, and machine-executable instructions are stored on memory 710. Alternatively, the code can be executed on a second computer system 740.

Aspects of the systems and methods provided herein, such as the server 701, can be embodied in programming. Various aspects of the technology may be thought of as “products” or “articles of manufacture” typically in the form of machine (or processor) executable code and/or associated data that is carried on or embodied in a type of machine readable medium (e.g., computer readable medium). Machine-executable code can be stored on an electronic storage unit, such memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless likes, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, tangible storage medium, a carrier wave medium, or physical transmission medium. Non-volatile storage media can include, for example, optical or magnetic disks, such as any of the storage devices in any computer(s) or the like, such may be used to implement the system. Tangible transmission media can include: coaxial cables, copper wires, and fiber optics (including the wires that comprise a bus within a computer system). Carrier-wave transmission media may take the form of electric or electromagnetic signals, or acoustic or light waves such as those generated during radio frequency (RF) and infrared (IR) data communications. Common forms of computer-readable media therefore include, for example: a floppy disk, a flexible disk, hard disk, magnetic tape, any other magnetic medium, a CD-ROM, DVD, DVD-ROM, any other optical medium, punch cards, paper tame, any other physical storage medium with patterns of holes, a RAM, a ROM, a PROM and EPROM, a FLASH-EPROM, any other memory chip or cartridge, a carrier wave transporting data or instructions, cables, or links transporting such carrier wave, or any other medium from which a computer may read programming code and/or data. Many of these forms of computer readable media may be involved in carrying one or more sequences of one or more instructions to a processor for execution.

### Example 5

### Media for Growing Bacteria Strains

A microbial strain of the invention can be grown using the media described in this example. For preparing the media, combine all ingredients shown in Table 7:

TABLE 7
Recipe for growth media

| PYGveg | | |
|---|---|---|
| Component | Amount per liter | |
| Glucose | 5 | g |
| K₂HPO₄ | 2 | g |
| Tween 80 | 1 | ml |
| Cystein-HCl | 0.5 | g |
| Yeast extract | 10 | g |
| HiVeg Extract | 5 | g |
| HiVeg Peptone #1 | 5 | g |
| HiVeg Peptone #3 | 5 | g |
| Vitamin Mix 100x | 10 | ml |
| Salt solution | 40 | ml |

| Salt solution | |
|---|---|
| Component | grams per liter |
| CaCl₂ 2H₂O | 0.02 |
| MgSO₄ 7H₂O | 0.02 |
| K₂HPO₄ | 0.04 |
| KH₂PO₄ | 0.04 |
| NaHCO₃ | 0.4 |
| NaCl | 0.08 |

Dissolve the ingredients in boiling water, which can contain less oxygen. Purge with nitrogen gas until the medium is completely anaerobic. Seal bottle with rubber septum. Let the medium cool down. Perform aliquoting of the anaerobic medium in a glove box to maintain anaerobic condition. Autoclave the medium for about 20 minutes at 121 degrees Celsius. Let the medium cool down and add the appropriate amount of 100× vitamins, shown in Table 8 below, to result in 1× final solution of growth medium.

TABLE 8
Vitamin solution

| Vitamin Solution | milligrams |
|---|---|
| Component | per liter |
| D-biotin | 0.2 |
| Ca-pantothenate | 2.5 |
| myoinositol | 20 |
| p-aminobenzoic acid | 0.5 |
| pyridoxine | 5 |
| hydrochloride | |
| riboflavine | 0.5 |
| thiamine dichloride | 10 |
| vitamin B12 | 0.2 |
| nicotinic acid | 5 |

### Example 6

### Preparation of Cells for Growth Assays

The example provides a protocol for preparing cells from a batch culture for growth assays.

Materials include sterile 48-well plate flat bottom; Sterile 384-well plate flat bottom; Sterile anoxic medium PYGveg with vitamins; Culture at OD of about 0.3 from freezer stock (e.g., serves as “standard”); samples to test (before and after treatment); 1250 ul multichannel multistep automatic pipette (e.g., E1-ClipTip™ Electronic Adjustable Tip Spacing Multichannel Equalizer Pipettes); Pipette tips (e.g., E1-ClipTip™); Sterile reservoirs; Breathe-Easy® sealing membrane (e.g., Sigma™ Z380059-1PAK); 70% Ethanol; Sterile syringe; Sterile needles; Sterile 50 ml conical tubes; Sterile 10 ml serological pipettes; Sterile pre weight 350 ml centrifugation container and lid; Sterile filtered cryoprotectant agent (CPA); Sterile 5 ml glass vial; Sterile grey rubber stopper; Sterile split rubber stopper; Freeze drying indicator; Sterile 48-well plates; sterile 96-well plates; Sterile reservoirs (i.e., unopened); Sterile pipette tips for automatic multichannel pipette (e.g., 1250 ul); Sterile 10 ml serological pipettes; Sterile 50 ml conical tubes; Sterile 1 ml syringe plunger.

Autoclaving can be performed at 121° C. for 20 minutes for sterilization.

20 mL of sterile filtered cryoprotectant agent (CPA) was prepared using, for example, 5% Trehalose, 5% Glycerol, 5% Sucrose, and 5% Inulin in PYG_{veg} medium.

10 plungers were wrapped in aluminum foil and autoclaved at 121° C. for 20 minutes. Plungers were allowed to dry for about 5-6 hours in an oven at 70° C.

Procedure:

A 400 ml culture from a 37° C. shaker incubator was transferred into an anaerobic box.

A blue rubber septum was sterilized using 70% ethanol. A 1 ml sample of the 400 ml culture was drawn by puncturing blue butyl rubber septum using a sterile needle attached to a sterile 1 ml syringe.

Two different wells of a 96-well plate were filled each with 200 μl of the culture. One of the two 200 μl samples was then diluted by 1:1 and 1:5 with media that is anaerobic and sterile into two additional wells. The measurements were performed in triplicate across the 3 dilutions (undiluted, 1:1, and 1:5) and averaged for accurate OD determination. This precise OD measure is to determine when the optimal moment to use the culture is (i.e. before entering into stationary phase).

When the culture reached late exponential phase (e.g., OD₆₀₀ of about 0.8), 25 ml of culture per condition (e.g., “before”, “after cryoprotectant 1 (CPA1)”, “after cryoprotectant 2 (CPA2)” etc.) were aliquoted using a 10 ml serological pipette into 50 ml centrifuge tubes and centrifuged at 12,000×g for 10 minutes. The supernatant was decanted and the pellet resuspended with 150 μl of media (e.g., for “before” samples) or a suitable cryoprotectant (e.g., CPA samples; equaled about 1 ml CPA/g of cells; different cryoprotectants can be tested). The homogenous suspension was transferred into a sterile glass vial and the vial closed with a grey rubber stopper. The vial was placed at −80° C. for a minimum of 2 hours. 500 μl of freeze drying indicator was added into a glass vial with grey rubber stopper and the vial stored with the samples at −80° C.

The remaining culture (e.g., culture left behind after 25 ml aliquots were taken) was poured into large centrifuge bottles and centrifuged at 12,000×g for 30 minutes. The supernatant was decanted and containers were placed upside down on paper towel to remove excessive liquid. Wet weight of the pellet was determined by weighing the centrifugation container including lid and cell pellet. The pellet was resuspended with a cryoprotectant (e.g., for about 1 g of pellet, about 1 ml of cryoprotectant was used). Weight of an empty glass vial with split rubber stopper and crimp cap was determined. The homogenous suspension was transferred into the empty glass vial with grey rubber stopper. The vial was placed in a freezer at −80° C. overnight. These cells were used later for testing the physical state of the powder after lyophilization.

After 2 hours, vials with frozen resuspended pellets from 25 ml samples were taken out from the −80° C. freezer and placed on dry ice. The grey rubber stoppers were replaced with sterile split stoppers. The stoppers were inserted halfway, e.g, to allow vapor to escape during lyophilization process. When lyophilizer reached appropriate vacuum and temperature (e.g., <0.01 mPA, −80 C), vials with samples were quickly transferred to a canister and inserted into the lyophilizer. The vial with the freeze dry indicator was also run alongside the vials with the samples. The freeze dry indicator was monitored for color change from pink to blue (e.g., pink=wet; blue=dry). Change of indicator from pink to blue can be indicative of lyophilization and drying of the bacterial samples. When the indicator changed from pink to blue, canisters with samples were taken out of the lyophilizer and the split stopper immediately closed. Photographs of the dried lyophilized samples were taken.

The dry lyophilized powder was resuspended in 2.5 ml of fresh media. Serial dilutions were then set up for SGT and Flow cytometry assays.

### Example 7

### Start Growth Time (SGT) Assay for Measuring Cell Growth and/or Viability of Cells

A Start Growth Time (SGT) assay can be based on the re-growth time that can be required by a growing cell culture, for example, to reach a threshold (e.g., threshold OD). This time can be proportional to the number of viable cells in the initial inoculum.

Day 1:

Protocol described in Example 5 was performed.

Using a reservoir and multichannel pipette, 900 ul of fresh medium was aliquoted into a 48-well plate.

Frozen glycerol stocks of SGT standards of respective strains (OD₆₀₀ of 0.3) were thawed inside a glove box (e.g., to maintain anaerobic conditions). The stocks were inverted at least 5 times to mix culture.

Thawed SGT standards and samples prepared in Example 5 (e.g., “before”, “CPA1”, “CPA2”, “CPA3”, “CPA4”) were serially diluted 1:10 into the 48-well plates (e.g., 100 μl into 900 μl media) to obtain 10⁻¹ to 10⁻⁵ dilutions of the standards (“Std”) and samples (e.g., “before”, “CPA1”, “CPA2”, “CPA3”, “CPA4”), and one well containing a blank (e.g., media only). Different cryoprotective agents (e.g., CPA1, CPA2, CPA3, CPA4) can be tested. Table 9 illustrates an example of a plate prepared for SGT.

TABLE 9
Illustrative 48-well plate for SGT.

| | A | B | C | D | E | F | G |
|---|---|---|---|---|---|---|---|
| 1 | Dilutions- | 10⁻¹ | 10⁻² | 10⁻³ | 10⁻⁴ | 10⁻⁵ | blank |
| 2 | Std | 900 ul + | | | | | |
| | | 100 ul | | | | | |
| 3 | Before | | | | | | |
| 4 | CPA1 | | | | | | |
| 5 | CPA2 | | | | | | |
| 6 | CPA3 | | | | | | |
| 7 | CPA4 | | | | | | |

Using the automatic multichannel pipettor, 90 μl of each condition (e.g., each duplicate of std, before, CPA1, CPA2, CPA3, CPA4 etc.) was transferred from the 48-well plate to a 384-well plate. For each condition in the 48-well plate, there were 4 replicates in the 384-well plate. Table 10 illustrates an example of a 384-well plate.

TABLE 10

| | A | B | C | D | E | F | G | H | I |
|---|---|---|---|---|---|---|---|---|---|
| 1 | Dilutions- | 10⁻¹ | 10⁻¹ | 10⁻² | 10⁻² | 10⁻³ | 10⁻³ | 10⁻⁴ | 10⁻⁴ |
| 2 | Std | 90 ul | 90 ul | | | | | | |
| 3 | Std | 90 ul | 90 ul | | | | | | |
| 4 | Before | | | | | | | | |
| 5 | Before | | | | | | | | |
| 6 | CPA1 | | | | | | | | |
| 7 | CPA1 | | | | | | | | |

The plate was incubated in a plate reader incubator at 37° C. for 48 h, with continuous double orbital shaking and reading intervals of 1 hour. Examples of growth curves measured using the SGT assay are illustrated in FIG. 8.

Day 2

The vial with frozen resuspended pellet from the rest of the culture was taken out from the −80° C. freezer and placed in a freeze dryer to lyophilize.

Day 3

Once lyophilized, the weight of the lyophilized sample from the rest of the culture was measured. The physical state of the lyophilized sample “cake” was analyzed and ground up to powder with sterilized 1 ml syringe plunger.

Determination of Viability

For the determination of viability, growth curves (e.g., average of 4 replicates) were plotted from each dilution of the standards and samples.

A Control (Ct) threshold was set at the maximum slope of sigmoidal curve of the Standards. Time for crossing the threshold was determined for the growing cell culture.

A standard curve was generated with Ct value as a function of cell/ml. The same wells of the standard were used to make a direct comparison between the growth curve analysis (SGT) and the flow cytometer count.

The Ct threshold was applied to the growth curves of the samples (e.g., “before”, “CPA1”, “CPA2”, “CPA3”, “CPA4”) and time to cross this threshold was determined for the growing cell culture.

The amounts of viable cells in the samples were determined using the standard curve equation.

### Example 8

### Assay for Measuring Cell Viability Using a Flow Cytometer

This example provides a protocol for an assay for quantitating live cells in a growth culture.

Materials

BD Cell Viability Kit Cat#349483; Carboxyfluorescein diacetate succinimidyl ester (cFDA) dye; 1×PBS; 0.01% Tween20; 96-well microtiter plate; and 70% isopropanol.

Procedure

Flow buffer (e.g., 1×PBS with 0.01% Tween20) was prepared.

The 48-well serial dilution plate prepared in Example 6 (e.g., Table 9) was also used for this assay. 100 μl of the highest concentrated cells from the serial dilution were used as follows: a) Tube 1 with 100 μl of treated cells (e.g., after lyophilization); b) Tube 2 of 100 μl of untreated cells (e.g., before lyophilization), and c) Tube 3 of 100 μl of untreated cells (e.g., before lyophilization) for control (e.g., as a negative “killed” control by treating the sample with isopropanol)

The tubes were centrifuged at 5000 rpm for 5 minutes. Cell pellet in each tube was resuspended in equal volume of pellets as follows: a) Tube 1 pellet (i.e. treated sample) was resuspended in flow buffer without dye; b) Tube 2 pellet (i.e. untreated sample) was resuspended in flow buffer without dye; and c) Tube 3 pellet (i.e. negative control of untreated sample) was resuspended in 70% isopropanol.

Tube 3 was incubated for 5 minutes at room temperature and centrifuged at 5000 rpm for 2 minutes to remove isopropanol. The isoproponal in the supernatant was aspirated while being careful not to disturb the pellet. The pellet was resuspended in an equal volume of flow buffer without dye.

To each tube containing treated, untreated, and negative control cells in flow buffer, 1 μl of Thiazole Orange (TO) and 1 μl of Propidium Iodide (PI) was added. The ratio of dye to buffer was 1:100. The tubes were vortexed to homogenize the samples and incubated at room temperature in the dark for about 10 minutes.

A 96-well plate was set up by aliquoting 90 μl of flow buffer into appropriate wells. 10 ul of the cells containing dye for the different samples were added to the 96-well plate and serial dilutions prepared as illustrated in Table 11.

TABLE 11
96-well plate for viability assay

| | A | B | C | D | E | F | G | H | I |
|---|---|---|---|---|---|---|---|---|---|
| 1 | | 1 | 2 | 3 | 4 | 5 | 6 | 7 | 8 |
| 2 | A | blank | most | less | blank | blank | most | less | blank |
| | | | dilute | dilute | | | dilute | dilute | |
| 3 | B | blank | most | less | blank | blank | most | less | blank |
| | | | dilute | dilute | | | dilute | dilute | |
| 4 | C | blank | most | less | blank | blank | most | less | blank |
| | | | dilute | dilute | | | dilute | dilute | |

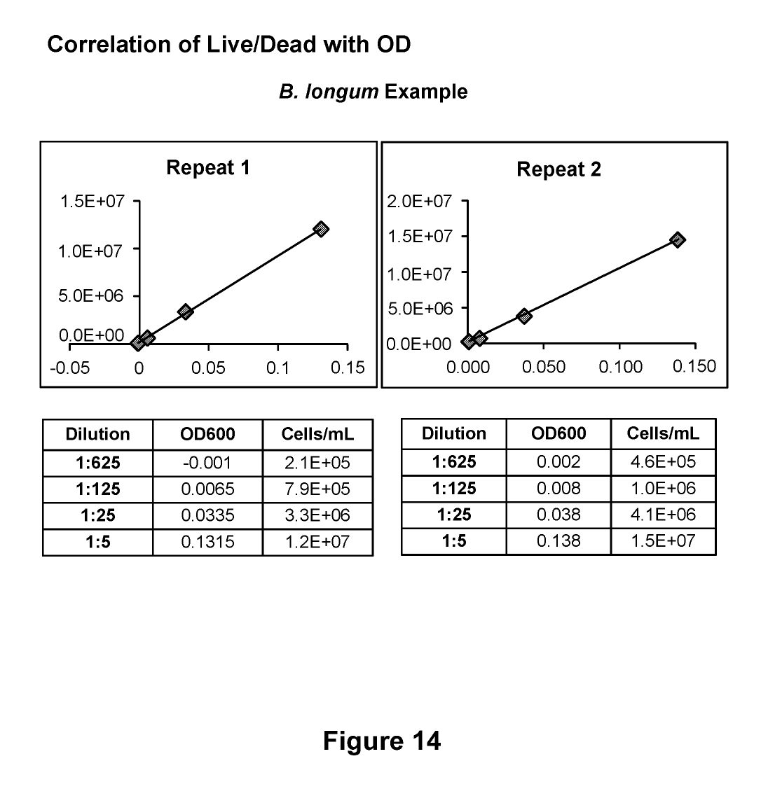

A flow cytometer was used to read the wells of the 96-well plate. FIG. 13 illustrates results of an assay to quantitate live cells. FIG. 14 illustrates an example of a correlation of live/dead cell ratio, determined from flow cytometer, with Optical Density (OD; e.g., OD₆₀₀) for the microbe B. longum.

### Example 9

### Treatment of a Skin Disorder with a Microbial Composition

A subject with a skin disorder, for example, atopic dermatitis comes to a medical professional for treatment.

The medical professional prescribes a microbial-based oral composition comprising the microbial strains Akkermansia muciniphila, Bifidobacterium adolescentis, Bifidobacterium infantis, Bifidobacterium longum, Clostridium beijerinckii, Clostridium butyricum, Clostridium indolis, and Eubacterium hallii. The composition may additionally comprise Faecalibacterium prausnitzii in some embodiments. Each strain is present in a range of about 10⁸ to about 10⁹ CFU in the composition. The composition additionally comprises inulin at a concentration of about 70 mg/mL in some embodiments. The expected delivery form of the oral composition is a pill with, for example, a pH-sensitive enteric coating (e.g., pH sensitive polymer, Eudragit FS30D) that can protect the contents from stomach acidity and deliver to the ileum/upper colon region of the subject. The enteric coating is designed to dissolve at a pH greater than about pH 6.5-7. In some embodiments, the oral composition is administered as a liquid capsule.

The subject is administered the composition before food intake (e.g., 1 hour before meals) twice daily for fourteen consecutive days. In some cases, the composition is administered simultaneously with food intake.

Following treatment, the subjects has a restored gut microbiome. The subject's symptoms associated with the skin disorder are completely treated.

### Example 10

### Microbial Compositions in Modulating Gut Microbiome to Treat a Skin Disorder (e.g. Atopic Dermatitis)

Background:

The gut microbiome can be involved with training the immune system. Dysbiosis of the gut microbiome can lead to many downstream inflammation-based reactions that can manifest in several ways, including on the skin as with atopic dermatitis. Correcting the gut dysbiosis can therefore alleviate downstream inflammation of the skin.

Objective:

The purpose of the study is to assess the effect of microbial compositions of the invention in restoring normal gut microbiome function and treat skin disorders/conditions.

Methods:

Twenty subjects with skin disease, enter a double-blind, placebo controlled and randomized study.
1) Experimental group: Ten subjects are given oral compositions containing the active composition comprising: Akkermansia muciniphila, Bifidobacterium adolescentis, Bifidobacterium infantis, Bifidobacterium longum, Clostridium beijerinckii, Clostridium butyricum, Clostridium indolis, and Eubacterium hallii. The composition can additionally comprise Faecalibacterium prausnitzii. The composition can additionally comprise inulin. The composition is taken once a day for 3 weeks. Parameters observed are number, rate, and severity of skin disorder related symptoms (e.g., atopic dermatitis breakouts).
2) Control group: Ten subjects are given a placebo pill. The placebo is taken once a day for 3 weeks. Parameters observed are number, rate, and severity of atopic dermatitis breakouts.

Following treatment, subjects in the experimental group have a restored gut microbiome and significantly lower number, rate, and severity of atopic dermatitis breakouts compared with the control group.

1. A method of treating a skin disorder in a subject in need thereof, the method comprising administering to the subject a pharmaceutical composition comprising a therapeutically-effective amount of a population of isolated and purified microorganisms.

2. The method of claim 1, wherein said population of isolated and purified microorganisms comprises a microorganism that modulates a pH of the subject.

3. The method of claim 2, wherein said microorganism modulates the pH of the skin of the subject.

4. The method of claim 1, wherein said skin disorder is atopic dermatitis.

5. The method of claim 1, wherein said population of isolated and purified microorganisms comprises a microorganism that produces vinegar.

6. The method of claim 1, wherein said population of isolated and purified microorganisms comprises a microorganism that produces hydrogen peroxide.

7. The method of claim 1, wherein said treating results in an altered microbiome in said subject.

8. The method of claim 1, wherein said treating results in a reduction of a pH of said subject.

9. The method of claim 8, wherein said reduction comprises reducing said pH to a range from about pH 4.0 to about pH 7.0.

10. The method of claim 1, wherein the pharmaceutical composition is formulated for oral administration.

11. The method of claim 1, wherein the pharmaceutical composition is formulated for topical administration.

12. The method of claim 1, wherein the pharmaceutical composition further comprises vinegar.

13. The method of claim 1, wherein the pharmaceutical composition further comprises trans-urocanic acid.

14. The method of claim 1, wherein the pharmaceutical composition further comprises a metabolite.

15. The method of claim 1, wherein the pharmaceutical composition further comprises an anti-microbial peptide.

16. The method of claim 1, wherein the pharmaceutical composition further comprises a bacteriocin.

17. The method of claim 1, wherein the pharmaceutical composition further comprises an enzyme.

18. The method of claim 1, wherein the pharmaceutical composition further comprises a prebiotic.

19. A pharmaceutical composition comprising a population of isolated and purified microorganisms, wherein said population of isolated and purified microorganisms comprises a microorganism that modulates pH.

20.-35. (canceled)

36. The method of claim 1, wherein said population of isolated and purified microorganisms comprises a microorganism with a rRNA sequence comprising at least about 85% sequence identity to a rRNA sequence of a microorganism selected from the group consisting of: Akkermansia muciniphila, Anaerostipes caccae, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium coccoides, Clostridium indolis, Clostridium nexile, Clostridium orbiscindens, Clostridium propionicum, Clostridium xylanolyticum, Enterococcus faecium, Eubacterium hallii, Eubacterium rectale, Faecalibacterium prausnitzii, Fibrobacter succinogenes, Lactobacillus acidophilus, Lactobacillus brevis, Lactobacillus bulgaricus, Lactobacillus casei, Lactobacillus caucasicus, Lactobacillus fermentum, Lactobacillus helveticus, Lactobacillus lactis, Lactobacillus plantarum, Lactobacillus reuteri, Lactobacillus rhamnosus, Oscillospira guilliermondii, Roseburia cecicola, Roseburia inulinivorans, Ruminococcus flavefaciens, Ruminococcus gnavus, Ruminococcus obeum, Stenotrophomonas nitritireducens, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus, Coprococcus eutactus, Eubacterium cylindroides, Eubacterium dolichum, Eubacterium ventriosum, Roseburia faeccis, Roseburia hominis, Roseburia intestinalis, Lacatobacillus bifidus, Lactobacillus johnsonii, and any combination thereof.

37. The method of claim 1, wherein said pharmaceutical composition further comprises hydrogen peroxide.

38. The method of claim 18, wherein said prebiotic is selected from the group consisting of: complex carbohydrate, complex sugar, resistant dextrin, resistant starch, amino acid, peptide, nutritional compound, biotin, polydextrose, oligosaccharide, polysaccharide, fructooligosaccharide (FOS), fructan, soluble fiber, insoluble fiber, fiber, starch, galactooligosaccharides (GOS), inulin, lignin, psyllium, chitin, chitosan, gums, high amylose cornstarch (HAS), cellulose, β-glucan, hemi-cellulose, lactulose, mannooligosaccharide, mannan oligosaccharide (MOS), oligofructose-enriched inulin, oligofructose, oligodextrose, tagatose, trans-galactooligosaccharide, pectin, resistant starch, and xylooligosaccharide (XOS), and any combination thereof.

39. The method of claim 18, wherein said prebiotic is a polysaccharide.

40. The method of claim 1, wherein the population of isolated and purified microorganisms comprises a microorganism that increases butyrate production in the subject.

41. The method of claim 1, wherein the population of isolated and purified microorganisms comprises a microorganism that modulates short-chain fatty acid production in the subject.
